(19) United States
(12) Patent Application Publication
(10) Pub. No.: US 2019/0175560 A1
(43) Pub. Date: Jun. 13, 2019

(54) MEBENDAZOLE CANCER THERAPIES AND METHODS OF USE

(71) Applicant: Shepherd Therapeutics, Inc., Natick, MA (US)

(72) Inventors: Jamie Dempsey BARBER, Hanover, MA (US); Katherine ARLINE, Cambridge, MA (US); William M. SIDERS, Franklin, MA (US); Johanne KAPLAN, Sherborn, MA (US)

(21) Appl. No.: 16/207,710

(22) Filed: Dec. 3, 2018

Related U.S. Application Data
(60) Provisional application No. 62/636,557, filed on Feb. 28, 2018.
(60) Provisional application No. 62/593,388, filed on Dec. 1, 2017.

(51) Int. Cl. A61K 31/4184; A61P 35/00; A61K 9/51; A61K 9/00; A61P 35/04; A61K 33/243; A61K 31/704; A61K 31/7048; A61K 31/675; A61K 31/513; A61K 31/7068; A61K 31/282; A61K 31/517; A61K 31/4745; A61K 31/337; A61K 31/506; A61K 31/44; A61K 31/475; A61N 5/10

(52) U.S. Cl. CPC A61K 31/4184; A61N 5/10; A61K 9/5153; A61K 9/5161; A61K 9/0053; A61K 9/0019; A61P 35/04; A61K 33/243; A61K 31/704; A61K 31/7048; A61K 31/675; A61K 31/513; A61K 31/7068; A61K 31/282; A61K 31/517; A61K 31/4745; A61K 31/337; A61K 31/506; A61K 31/44; A61K 31/475; A61P 35/00

(57) ABSTRACT

The disclosure relates to a method of treating cancer by administering to the subject a therapeutically effective amount of a composition comprising mebendazole.

### RELATED APPLICATIONS

This application claims priority to U.S. Application No. 62/593,388 filed on Dec. 1, 2017 and U.S. Application No. 62/636,557 filed on Feb. 28, 2018, the contents of each of which are herein incorporated by reference in their entirety.

### FIELD OF THE DISCLOSURE

The disclosure relates to the fields of molecular biology, oncology and human therapeutics for the treatment of cancer.

### BACKGROUND

Cancer is a proliferative disease in which the cells of a subject grow abnormally and in an uncontrolled way, in some cases leading to the death of the subject with cancer. There are many independent events and causes which can lead to cancer, and many different cell types and tissues that can give rise to cancers. As such, treatments developed for one type of cancer may not work on another type of cancer. Despite many years of research, and a plethora of treatments available to cancer sufferers, there is still a long felt need in the art for additional cancer therapies. This need is particularly acute in rare cancers, which in many cases may be under-resourced because of their rarity. The disclosure provides additional methods for the treatment of cancer.

### SUMMARY

The disclosure provides methods of treating a cancer in a subject in need thereof, comprising administering to the subject a therapeutically effective amount of a composition comprising methyl N-(6-benzoyl-1H-benzimidazol-2-yl)carbamate (mebendazole).

The disclosure provides compositions for use in treating cancer in a subject in need thereof comprising a therapeutically effective amount of a composition comprising mebendazole.

The disclosure provides compositions for use in the manufacture of a medicament for the prevention or treatment of cancer comprising a therapeutically effective amount of a composition comprising mebendazole.

In some embodiments of the methods or compositions for use of the disclosure, the composition comprising mebendazole comprises a mebendazole hydrochloride salt ((5-benzoyl-1H-benzimidazole-2-yl)-carbamic acid methyl ester hydrochloride, MBZ.HCl), a mebendazole hydrobromide salt, a mebendazole maleate salt, a mebendazole glutarate salt, a mebendazole monomethyl oxalate salt or a mebendazole mesylate monohydrate. In some embodiments, the composition comprising mebendazole comprises a crystal polymorph A of mebendazole, a crystal polymorph B of mebendazole, a crystal polymorph C of mebendazole or a combination thereof. In some embodiments, the mebendazole comprises crystal polymorph C.

In some embodiments of the methods or compositions for use of the disclosure, the composition comprising mebendazole comprises a salt. In some embodiments, the mebendazole salt comprises a mebendazole hydrochloride salt, a mebendazole hydrobromide salt, a mebendazole maleate salt, a mebendazole-glutarate salt or a mebendazole monomethyl oxalate salt. In some embodiments, the composition comprising mebendazole comprises a crystal polymorph of mebendazole. In some embodiments, the crystal polymorph comprises a crystal polymorph A of mebendazole, a crystal polymorph B of mebendazole, a crystal polymorph C of mebendazole or a combination thereof. In some embodiments, the polymorph comprises crystal polymorph C.

In some embodiments of the methods or compositions for use of the disclosure, the composition comprising mebendazole further comprises a nanoparticle. In some embodiments, the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a diblock copolymer, a polymeric micelle or a hyperbranched macromolecule. In some embodiments, the polymer-based nanoparticle comprises a multiblock copolymer a diblock copolymer. In some embodiments, the polymer-based nanoparticle comprises a poly(lactic-co-glycolic acid) PLGA polymer. In some embodiments, the polymer-based nanoparticle is pH responsive. In some embodiments, the polymer-based nanoparticle further comprises a buffering component.

In some embodiments of the methods or compositions for use of the disclosure, the nanoparticle further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment. In some embodiments, the targeting agent binds to the surface of a cell of the cancer of the subject. In some embodiments, the targeting agent comprises hyaluronic acid (HA). In some embodiments, the HA binds to CD44 on the surface of a cancer cell of the subject.

In some embodiments of the methods or compositions for use of the disclosure, the cancer comprises a colorectal cancer, a gastric cancer, a brain cancer, a colon cancer, a breast cancer, a liver cancer, a lung cancer, a pancreatic cancer or a renal cancer. In some embodiments, the lung cancer comprises a small cell lung cancer or a non-small cell lung cancer.

In some embodiments of the methods or compositions for use of the disclosure, the cancer is a rare cancer. In some embodiments, the cancer is a blastoma, a sarcoma, a carcinoma, a neuroendocrine cancer, a mesothelioma, a chordoma, a thymic cancer, a gastrointestinal stromal tumor or a pheochromocytoma. In some embodiments, the sarcoma comprises an Ewing's sarcoma, a leiomyosarcoma, an angiosarcoma or a rhabdomyosarcoma. In some embodiments, the carcinoma comprises an adenoid cystic carcinoma (ACC), a uterine serous carcinoma, an adrenocortical carcinoma, a gastric carcinoma, a cholangiocarcinoma, a colorectal carcinoma, an esophageal carcinoma, a hepatocellular carcinoma, a pancreatic carcinoma, a small cell lung carcinoma, an ovarian carcinoma or a thymic carcinoma. In some embodiments, the adenoid cystic carcinoma (ACC) comprises a salivary gland cell, a trachea cell, a lacrimal gland cell, a breast cell, a skin cell or a vulval cell.

In some embodiments of the methods or compositions for use of the disclosure, the cancer is a rare cancer. In some embodiments, the cancer is a blastoma, a sarcoma, a carcinoma, a neuroendocrine cancer, a mesothelioma, a chordoma or a thymic cancer. In some embodiments, the blastoma comprises a neuroblastoma or a glioblastoma.

In some embodiments of the methods or compositions for use of the disclosure, the cancer is a rare cancer. In some embodiments, the cancer is a blastoma, a sarcoma, a carcinoma, a neuroendocrine cancer, a mesothelioma, a chordoma or a thymic cancer. In some embodiments, the thymic cancer comprises a thymoma or a thymic carcinoma. In some embodiments, the neuroendocrine cancer comprises a carcinoid tumor or a thymic cancer. In some embodiments, the carcinoid tumor comprises a small intestine tumor, an appendix tumor, a tumor of the rectum, a tumor of the bronchial system, a brain tumor, colon tumor, a stomach tumor, a pancreatic tumor, a liver tumor, a gallbladder tumor, a bile duct tumor, an ovarian tumor, a testicular tumor, a bladder tumor, a tumor of the prostate gland, a breast tumor, a kidney tumor, a thymic tumor, an eye tumor, an ear tumor or an adrenal tumor.

In some embodiments of the methods or compositions for use of the disclosure, the cancer is a stage 0 or stage 1 (early stage, pre-metastatic) cancer. In some embodiments, the cancer is a stage 2 cancer or stage 3 (spread to nearby tissues and lymph nodes) cancer. In some embodiments, the cancer is a stage 4 (advanced or metastatic) cancer.

In some embodiments of the methods of the disclosure, the subject is a mammal, a non-human primate or a human. In some embodiments, the subject is human. In some embodiments, the human is a male, a female, a child, a baby or a neonate.

In some embodiments of the methods or compositions for use of the disclosure, the composition comprising mebendazole is administered systemically or is suitable for systemic administration. In some embodiments, the composition comprising mebendazole is suitable for systemic, oral or parenteral administration. In some embodiments, the administration comprises at least 30 mg, at least 50 mg, at least 100 mg, at least 200 mg, at least 300 mg, at least 400 mg, at least 500 mg, at least 600 mg, at least 700 mg, at least 800 mg at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg or at least 2000 mg of mebendazole per day. In some embodiments, the administration comprises oral administration. In some embodiments, the oral administration occurs with food. In some embodiments, the administration occurs once a day. In some embodiments, the administration occurs twice a day. In some embodiments, the administration occurs three times a day. In some embodiments, the administration occurs four or more times a day.

In some embodiments of the methods or compositions for use of the disclosure, the composition comprising mebendazole is administered systemically or is suitable for systemic administration In some embodiments, the administration comprises at least 30 mg, at least 50 mg, at least 100 mg, at least 200 mg, at least 300 mg, at least 400 mg, at least 500 mg, at least 600 mg, at least 700 mg, at least 800 mg at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg or at least 2000 mg of mebendazole per day. In some embodiments, the composition comprising mebendazole is administered parenterally or is suitable for parenteral administration. In some embodiments, the parenteral administration comprises intramuscular, subcutaneous or intravenous administration. In some embodiments, the administration occurs once a day. In some embodiments, the administration occurs twice a day. In some embodiments, the administration occurs three times a day. In some embodiments, the administration occurs four or more times a day.

In some embodiments of the methods or compositions for use of the disclosure, the method of treatment or composition for use further comprises an additional cancer treatment. In some embodiments, the additional cancer treatment comprises a surgical procedure to remove at least one tumor of the cancer or at least one dose of a radiation therapy.

In some embodiments of the methods or compositions for use of the disclosure, the additional cancer treatment comprises a second chemotherapeutic agent, a combination chemotherapy, a therapeutic antibody, a chimeric antigen receptor T cell (CAR-T) therapy or a combination thereof. In some embodiments, the CAR-T therapy comprises Tisagenlecleucel (Kymriah™). In some embodiments, the second chemotherapeutic agent comprises a cell cycle checkpoint inhibitor, a CDK inhibitor, an mTOR inhibitor, an immune checkpoint modulator, an antimitotic agent, a pro-apoptotic agent, a DNA damaging agent or an inhibitor of a DNA damage response pathway. In some embodiments, the immune checkpoint modulator comprises Yervoy (Ipilimumab), Opdivo (Nivolumab), Tecentriq (Atezolizumab) or Keytruda (Pembrolizumab). In some embodiments, the CDK inhibitor comprises an inhibitor of CDK4, an inhibitor of CDK6 or an inhibitor of CDK4 and CDK6. In some embodiments, the CDK inhibitor comprises Abemaciclib (Verzenio), Palbociclib (Ibrance) or Ribociclib (Kisqali). In some embodiments, the mTOR inhibitor comprises Rapamycin (Sirolimus), Temsirolimus (Torisel), Everolimus (Afinitor) or Ridaforolimus.

In some embodiments of the methods or compositions for use of the disclosure, the method of treatment or composition for use further comprises an additional cancer treatment. In some embodiments, the additional cancer treatment comprises a second chemotherapeutic agent, a therapeutic antibody, a CAR-T therapy or a combination thereof. In some embodiments, the second chemotherapeutic agent comprises Abitrexate (Methotrexate), Afinitor (Everolimus), Alimta (PEMETREXED), Alkeran (Melphalan), Aredia (Pamidronate), Arimidex (Anastrozole), Aromasin (Exemestane), Arranon (Nelarabine), Beleodaq (Belinostat), BiCNU (Carmustine), Blenoxane (Bleomycin), Bosulif (Bosutinib), Busulfex (Busulfan), Caprelsa (Vandetanib), Carboplatin, Casodex (Bicalutamide), CeeNU (Lomustine), Cerubidine (Daunorubicin), Cisplatin, Clolar (Clofarabine), Cometriq (Cabozantinib), Cosmegen (Dactinomycin), Cotellic (Cobimetinib), CytosarU (Cytarabine), Cytoxan, Dacarbazine, Dacogen (Decitabine), DaunoXome (Daunorubicin Lipid Complex), Decadron (Dexamethasone), Docetaxel, Doxorubicin, DepoCyt (Cytarabine Lipid Complex), Dexamethasone Intensol (Dexamethasone), Dexpak Taperpak (Dexamethasone), Droxia (Hydroxyurea), Eligard (Leuprolide), Ellence (Epirubicin), Eloxatin (Oxaliplatin), Elspar (Asparaginase), Emcyt (Estramustine), Erivedge (Vismodegib), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), Fludara (Fludarabine), 5-Fluorouracil, Folex (methotrexate), Folotyn (Pralatrexate Injection), FUDR (floxuridine), Gemzar (Gemcitabine), Gilotrif (Afatinib), Gleevec (Imatinib Mesylate), Gliadel (Carmustine), HDAC (high dose cytarabine), Halaven (Eribulin), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Ifex (Ifosfamide), Imbruvica (Ibrutinib), Inlyta (Axitinib), Intron A alfab (Interferon alfa-2a), Iressa (Gefitinib), Irinotecan, Istodax (Romidepsin), Ixempra (Ixabepilone), Jakafi (Ruxolitinib), Jevtana (Cabazitaxel Injection), Kyprolis (Carfilzomib), Lenvima (Lenvatinib mesylate), Somatuline Depot (Lanreotide acetate), Leukeran (Chlorambucil), Leukine (Sargramostim), Leustatin (Cladribine), Lonsurf (Trifluridine and Tipiracil), Lupron (Leuprolide), Lupron Depot (Leuprolide), Lupron DepotPED (Leuprolide), Lynparza (Olaparib), Lysodren (Mitotane), Matulane (Procarbazine), Xofigo (Radium 223 dichloride), Megace (Megestrol), Mekinist (Trametinib), Mesnex (Mesna), Mesnex (Mesna Injection), Metastron (Strontium-89 Chloride), Mexate (Methotrexate) Mustargen (Mechlorethamine), Mutamycin (Mitomycin), Myleran (Busulfan), Navelbine (Vinorelbine), Neosar (Cyclophosphamide), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Odomzo (Sonidegib), Oncaspar (Pegaspargase), Ontak (Denileukin Diftitox), Paclitaxel, Panretin (Alitretinoin), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), RoferonA alfaa (Interferon alfa-2a), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b), Synribo (Omacetaxin), Tabloid (Thioguanine), Taflinar (Dabrafenib), Tarceva (Erlotinib), Targretin (Bexarotene), Dacarbazine, Temodar (Temozolomide), Tepadina (Thiotepa), Thalomid (Thalidomide), TheraCys BCG (BCG), Thioplex (Thiotepa), TICE BCG (BCG), Toposar (Etoposide), Torisel (Temsirolimus), Yondelis (Trabectedin), Treanda (Bendamustine hydrochloride), Trelstar (Triptorelin), Trexall (Methotrexate), Trisenox (Arsenic trioxide), Tykerb (lapatinib), Valstar (Valrubicin Intravesical), Vantas (Histrelin Implant), Velcade (Bortezomib), Vepesid (Etoposide), Vesanoid (Tretinoin), Vincristine, Vidaza (Azacitidine), Vinblastine, Votrient (Pazopanib), Vumon (Teniposide), Wellcovorin IV (Leucovorin), Xalkori (Crizotinib), Xeloda (Capecitabine), Xtandi (Enzalutamide), Zaltrap (Ziv-aflibercept), Zanosar (Streptozocin), Zelboraf (Vemurafenib), Zoladex (Goserelin), Zolinza (Vorinostat), Zometa (Zoledronic acid), Zortress (Everolimus), Zydelig (Idelalisib), Zykadia (Ceritinib), Zytiga (Abiraterone acetate), Vindesine (Eldesine), Raltitrexed (Tomudex), Lometrexol, Satraplatin, Larotaxel, Alectinib (Alecensa), Ixazomib (Ninlaro), Nilotinib (Tasigna), Osimertinib (Tagrisso), Venetoclax (Venclexta), Ribociclib (Kisqali), Enasidenib (Idhifa), Rucaparib (Rubraca), Niraparib (Zejula), Copanlisib (Aliqopa), Neratinib (Nerlynx), Brigatinib (Alunbrig), Midostaurin (Rydapt), Abemaciclib (Verzenio), Rapamycin (Sirolimus), Temsirolimus (Torisel), Ridaforolimus or a combination thereof.

In some embodiments of the methods or compositions for use of the disclosure, the method of treatment or composition for use further comprises an additional cancer treatment. In some embodiments, the additional cancer treatment comprises a surgical procedure to remove at least one tumor of the cancer, at least one dose of a radiation therapy, a second chemotherapeutic agent, a therapeutic antibody, a CAR-T therapy or a combination thereof. In some embodiments, the second chemotherapeutic agent comprises Paclitaxel, Docetaxel, Vinblastine, Vincristine, Cisplatin, Carboplatin, Oxaliplatin, Doxorubicin, Etoposide, Imatinib, Gemcitabine, Vinorelbine, Ifosamide, Abemaciclib, Sorafenib, Irinotecan, 5-Fluorouracil, Dacarbazine, Trabectedin, Temozolomide, Cyclophosphamide or a combination thereof.

In some embodiments of the methods or compositions for use of the disclosure, the method of treatment or composition for use further comprises an additional cancer treatment. In some embodiments, the additional cancer treatment comprises a surgical procedure to remove at least one tumor of the cancer, at least one dose of a radiation therapy, a second chemotherapeutic agent, a therapeutic antibody, a CAR-T therapy or a combination thereof. In some embodiments, the therapeutic antibody comprises Adcetris (Brentuximab Vedotin), Arzerra (Ofatumumab), Avastin (Bevacizumab), Bexxar (Tositumomab), Bavencio (Avelumab), Blincyto (Blinatumomab), Campath (Alemtuzumab), Cyramza (Ramucirumab), Darzalex (Daratumumab), Empliciti (Elotuzumab), Erbitux (Cetuximab), Gazyva (Obinutuzumab), Imfinzi (Durvalumab), Herceptin (Trastuzumab), Gazyvaro (Obinutuzumab), Kadcyla (Ado-trastuzumab Emtansine), Keytruda (Pembrolizumab), Lartruvo (Olaratumab), Mylotarg (Gemtuzumab Ozogamicin), Ocrevus (Ocrelizumab), Opdivo (Nivolumab), Perjeta (Pertuzumab), Portrazza (Necitumumab), Proxinium (Catumaxomab), Removab (Catumaxomab), Rituxan (Rituximab), Sylvant (Siltuximab), Tecentriq (Atezolizumab), Unituxin (Dinutuximab), Vectibix (Panitumumab), Yervoy (Ipilimumab), Xgeva (Denosumab), Zevalin (Ibritumomab Tiuxetan), Mogamulizumab (Poteligeo) or a combination thereof.

In some embodiments of the methods or compositions for use of the disclosure, the method of treatment or composition for use further comprises an additional cancer treatment. In some embodiments, the additional cancer treatment comprises a combination chemotherapy. In some embodiments, the combination chemotherapy comprises 7+3, ABVD, AC, AD, ADE, ADOC, BEACOPP, BEP, CAF, CAPIRI, CAPOX, CB, CBI, CEF, CEPP, CFAR, CHOP, CIM, CLAG, CLAG-M, CMC, CMF, COI, CVD, CVP, DHAP, DVD, ECF, ECX, EOF, EOX, EP, EPOCH, EPOCH+R, ESHAP, FAMTX, FC, FCR, FEC, FLAG-IDA, FLO, FLOX, FOLFIRI, FOLFOX, FOLFOXIRI, GEMOX-B, GVD, Hyper-CVAD, ICE, ICE-V, IFL, IROX, LV5FU2, LV5FU-P, MAID, MFL, MINE, MOPP, MP, MPV, MVAC, OFF, PAC, PAD, PCR, PCV, R-MPV, R-GemOx, R-CHOP, R-CVP, R-FCM, RICE, TAC, TC, TCH, TIP, TPC, TPF, VAD, VIP, VMP, VMPT, XELIRI or XELOX.

In some embodiments of the methods or compositions for use of the disclosure, the composition comprising mebendazole and the additional cancer treatment are in the same composition. In some embodiments, the composition comprising mebendazoleis formulated in a nanoparticle. In some embodiments, the composition comprising mebendazole and the additional cancer treatment are formulated in a nanoparticle.

In some embodiments of the methods or compositions for use of the disclosure, the additional cancer treatment and the composition comprising mebendazole are suitable for simultaneous administration. In some embodiments, the additional cancer treatment and the composition comprising mebendazole are suitable for sequential administration. In some embodiments, the additional cancer treatment and the composition comprising mebendazole are suitable for administration in temporal proximity.

In some embodiments of the methods or compositions for use of the disclosure, the additional cancer treatment and the composition comprising mebendazole exhibit synergy. In some embodiments, the synergy is measured using the Chou-Talalay method in at least one cancer cell line. In some embodiments, the synergy comprises a CI of less than 0.9 when measured at at least three concentrations of the additional cancer treatment and the composition comprising mebendazole in at least one cancer cell line. In some embodiments, the composition comprising mebendazole and the additional cancer treatment are each suitable for administration in a syngergistically effective amount.

In some embodiments of the methods or compositions for use of the disclosure, the method of treatment or composition for use alleviates a sign or a symptom of the cancer. In some embodiments, the alleviation of the sign or the symptom of the cancer comprises a reduction in size of at least one tumor, a reduction in the volume of at least one tumor, a decrease in the number of tumors, a decrease in the number of metastatic lesions of the cancer, a reduction of the rate of growth of the cancer or a remission of the cancer.

The disclosure provides compositions comprising a synergistic combination of mebendazole and at least one additional cancer therapeutic agent.

In some embodiments of the compositions of the disclosure, the synergy is measured using the Chou-Talalay method in at least one cancer cell line. In some embodiments, the synergy comprises a CI of less than 0.9 when measured at at least three concentrations of the additional cancer therapeutic agent and the composition comprising mebendazole in at least one cancer cell line.

In some embodiments of the compositions of the disclosure, the mebendazole comprises a salt. In some embodiments, the mebendazole salt comprises a mebendazole hydrochloride salt, a mebendazole hydrobromide salt, a mebendazole maleate salt, a mebendazole-glutarate salt or a mebendazole monomethyl oxalate salt. In some embodiments, the mebendazole comprises a crystal form or a polymorph of mebendazole. In some embodiments, the polymorph comprises a polymorph A of mebendazole, a polymorph B of mebendazole, a polymorph C of mebendazole or a combination thereof. In some embodiments, the polymorph comprises crystal polymorph C.

In some embodiments of the compositions of the disclosure, the at least one additional cancer therapeutic agent comprises Cisplatin, Doxorubicin, Etoposide, Cyclophosphamide, 5-FU, Gemcitabine, Oxaliplatin, Irinotecan, Vinorelbine, Dacarbazine, Vincristine, Sorafenib, Paclitaxel, Imatinib, Abemaciclib, Ifosamide or Docetaxel.

In some embodiments of the compositions of the disclosure, the mebendazole is formulated in a nanoparticle. In some embodiments, the mebendazole and the at least one additional cancer therapeutic agent are formulated in a nanoparticle. In some embodiments of the compositions of the disclosure, the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a diblock copolymer, a polymeric micelle or a hyperbranched macromolecule. In some embodiments, the polymer-based nanoparticle comprises a multiblock copolymer a diblock copolymer. In some embodiments, the polymer-based nanoparticle comprises a poly(lactic-co-glycolic acid) PLGA polymer.

In some embodiments of the compositions of the disclosure, the polymer-based nanoparticle is pH responsive.

In some embodiments of the compositions of the disclosure, the polymer-based nanoparticle further comprises a buffering component.

In some embodiments of the compositions of the disclosure, the nanoparticle further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment. In some embodiments, the targeting agent comprises hyaluronic acid (HA). In some embodiments, the targeting agent binds to the surface of a cell of the cancer of the subject.

The disclosure provides combinational therapies for treating cancer comprising administering a therapeutically effective amount of the compositions of the disclosure to a subject in need thereof.

The disclosure provides combinational therapies for treating cancer comprising administering a synergistically effective amount of the compositions of the disclosure to a subject in need thereof.

The disclosure provides compositions for use in a combinational therapy to treat cancer, comprising a therapeutically effective amount of the compositions comprising mebendazole of the disclosure.

The disclosure provides compositions for use in a combinational therapy, wherein the combinational therapy comprises administering one or more additional cancer therapies to the subject.

The disclosure provides kits comprising the compositions of the disclosure and instructions for use in the treatment of cancer.

The disclosure provides kits comprising a therapeutically effective amount of a composition comprising mebendazole and instructions for use in the treatment of cancer. In some embodiments of the kits of the disclosure, the kit further comprises at least one additional cancer therapeutic agent. In some embodiments, the therapeutically effective amount of the composition comprising mebendazole comprises a synergistically effective amount of the composition comprising mebendazole. In some embodiments, the composition comprising mebendazole and the at least one additional cancer therapeutic agent exhibit synergy. In some embodiments, the additional cancer therapeutic agent comprises a second chemotherapeutic agent, a combination chemotherapy, a therapeutic antibody, a chimerica antigen T cell (CAR-T) therapy or a combination thereof. In some embodiments, the at least one additional cancer therapeutic comprises Cisplatin, Doxorubicin, Etoposide, Cyclophosphamide, 5-FU, Gemcitabine, Oxaliplatin, Irinotecan, Vinorelbine, Dacarbazine, Vincristine, Sorafenib, Paclitaxel, Imatinib, Abemaciclib, Ifosamide or Docetaxel. In some embodiments, the mebendazole is formulated in a nanoparticle. In some embodiments, the mebendazole and the at least one additional cancer therapeutic are formulated in a nanoparticle. In some embodiments, the nanoparticle comprises a PLGA polymer and an HA targeting agent.

### BRIEF DESCRIPTION OF THE DRAWINGS

FIG. 1A is a plot showing the effect of mebendazole (MBZ) at 24 hours on viability for A2780cis, SKOV-3 and TOV-112D cells treated with increasing concentrations of MBZ.

FIG. 1B is a plot showing the effect of mebendazole (MBZ) at 48 hours on viability for A2780cis, SKOV-3 and TOV-112D cells treated with increasing concentrations of MBZ.

FIG. 1C is a plot showing the effect of mebendazole (MBZ) at 72 hours on viability for A2780cis, SKOV-3 and TOV-112D cells treated with increasing concentrations of MBZ.

FIG. 2 is a plot showing the effect of mebendazole treatment on SKOV-3 ovarian cell line cells treated for 72 hours.

FIG. 3A is a plot showing the effect of mebendazole treatment on TC-71 Ewing's sarcoma cell line cells treated for 72 hours. On the X-axis, the log concentration of mebendazole in μM, indicated from −4 to 2 in units of 2. On the Y-axis, percent viability, from 0 to 100 in units of 50.

FIG. 3B is a plot showing the effect of mebendazole treatment on TC-32 Ewing's sarcoma cell line cells treated for 72 hours. On the X-axis, the log concentration of mebendazole in μM, indicated from −4 to 2 in units of 2. On the Y-axis, percent viability, from 0 to 100 in units of 50.

FIG. 3C is a plot showing the effect of mebendazole treatment on CHLA-9 Ewing's sarcoma cell line cells treated for 72 hours. On the X-axis, the log concentration of mebendazole in μM, indicated from −4 to 2 in units of 2. On the Y-axis, percent viability, from 0 to 150 in units of 50.

FIG. 4A is a plot showing the effect of mebendazole treatment on IMR-32 Neuroblastoma cell line cells treated for 72 hours. On the X-axis, the log concentration of mebendazole in μM, indicated from −4 to 2 in units of 2. On the Y-axis, percent viability, from 0 to 125 in units of 25.

FIG. 4B is a plot showing the effect of mebendazole treatment on CHP-212 Neuroblastoma cell line cells treated for 72 hours. On the X-axis, the log concentration of mebendazole in μM, indicated from −4 to 1 in units of 1. On the Y-axis, percent viability, from 0 to 125 in units of 25.

FIG. 4C is a plot showing the effect of mebendazole treatment on SK-N-AS Neuroblastoma cell line cells treated for 72 hours. On the X-axis, the log concentration of mebendazole in μM, indicated from −4 to 2 in units of 1. On the Y-axis, percent viability, from 0 to 150 in units of 50.

FIG. 5 is a plot showing the effect of mebendazole treatment on SK-UT-1B Leiomyosarcoma cell line cells treated for 72 hours. On the X-axis, the log concentration of mebendazole in μM, indicated from −4 to 2 in units of 1. On the Y-axis, percent viability, from 0 to 150 in units of 50.

FIG. 6A is a plot showing the effect of mebendazole treatment on SW-13 Adrenal Cortical Carcinoma (ACC) cell line cells treated for 72 hours. On the X-axis, the log concentration of mebendazole in μM, indicated from −4 to 3 in units of 1. On the Y-axis, percent viability, from 0 to 150 in units of 50.

FIG. 6B is a plot showing the effect of mebendazole treatment on NCI-H295R Adrenal Cortical Carcinoma (ACC) cell line cells treated for 72 hours. On the X-axis, the log concentration of mebendazole in μM, indicated from −4 to 4 in units of 2. On the Y-axis, percent viability, from 0 to 125 in units of 25.

FIG. 7A is a plot showing the effect of mebendazole treatment on Rh-30 Rhabdomyosarcoma cell line cells treated for 72 hours. On the X-axis, the log concentration of mebendazole in μM, indicated from −4 to 2 in units of 2. On the Y-axis, percent viability, from 0 to 125 in units of 25.

FIG. 7B is a plot showing the effect of mebendazole treatment on Rh-41 Rhabdomyosarcoma cell line cells treated for 72 hours. On the X-axis, the log concentration of mebendazole in μM, indicated from −4 to 2 in units of 2. On the Y-axis, percent viability, from 0 to 125 in units of 25.

FIG. 8A is a plot showing the effect of mebendazole treatment on U-CH2 chordoma cell line cells treated for 72 hours. On the X-axis, the log concentration of mebendazole in μM, indicated from −4 to 3 in units of 1. On the Y-axis, percent viability, from 0 to 150 in units of 50.

FIG. 8B is a plot showing the effect of mebendazole treatment on Mug-Chort1 chordoma cell line cells treated for 72 hours. On the X-axis, the log concentration of mebendazole in μM, indicated from −4 to 3 in units of 1. On the Y-axis, percent viability, from 0 to 150 in units of 50.

FIG. 9A is a plot showing the effect of mebendazole treatment on KATO-III gastric carcinoma cell line cells treated for 72 hours. On the X-axis, the log concentration of mebendazole in μM, indicated from −4 to 4 in units of 2. On the Y-axis, percent viability, from 0 to 150 in units of 50.

FIG. 9B is a plot showing the effect of mebendazole treatment on SNU-16 gastric carcinoma cell line cells treated for 72 hours. On the X-axis, the log concentration of mebendazole in μM, indicated from −4 to 4 in units of 2. On the Y-axis, percent viability, from 0 to 150 in units of 50.

FIG. 9C is a plot showing the effect of mebendazole treatment on NCI-N87 carcinoma cell line cells treated for 72 hours. On the X-axis, the log concentration of mebendazole in μM, indicated from −4 to 4 in units of 2. On the Y-axis, percent viability, from 0 to 150 in units of 50.

FIG. 10A is a plot showing the effect of the vehicle alone control on tumor size in Adenoid Cystic Carcinoma (ACC) ACCX6 patient derived xenograft mice. The X-axis shows day of treatment, while the Y-axis in shows tumor size in mm³.

FIG. 10B is a plot showing the effect of the 50 mg/kg/day of mebendazole on tumor size in Adenoid Cystic Carcinoma (ACC) ACCX6 patient derived xenograft mice. The X-axis shows day of treatment, while the Y-axis in shows tumor size in mm³.

FIG. 10C is a plot showing the effect of the 200 mg/kg/day of mebendazole on tumor size in Adenoid Cystic Carcinoma (ACC) ACCX6 patient derived xenograft mice. The X-axis shows day of treatment, while the Y-axis in shows tumor size in mm³.

FIG. 10D is a plot showing the effect on mean tumor size for the vehicle treated mice (black circles), 50 mg/kg/day MBZ treated mice (squares) and 200 mg/kg/day MBZ treated mice (triangles) with ACCX6C patient derived xenograft tumors. The X-axis shows day of treatment, while the Y-axis in shows tumor size in mm³.

FIG. 11 is a plot showing the survival ACCX6C patient derived xenograft mice treated with vehicle, 50 mg/kg/day MBZ and 200 mg/kg/day MBZ.

FIG. 12A is a plot showing the effect of the vehicle alone control on tumor size in ACCX9 patient derived xenograft mice. The X-axis shows day of treatment, while the Y-axis in shows tumor size in mm³.

FIG. 12B is a plot showing the effect of the 50 mg/kg/day of mebendazole on tumor size in ACCX9 patient derived xenograft mice. The X-axis shows day of treatment, while the Y-axis in shows tumor size in mm³.

FIG. 12C is a plot showing the effect of the 200 mg/kg/day of mebendazole on tumor size in ACCX9 patient derived xenograft mice. The X-axis shows day of treatment, while the Y-axis in shows tumor size in mm³.

FIG. 12D is a plot showing the effect on mean tumor size for the vehicle treated mice (circles), 50 mg/kg/day MBZ treated mice (squares) and 200 mg/kg/day MBZ treated mice (triangles) with ACCX9 patient derived xenograft tumors. The X-axis shows day of treatment while the Y-axis in shows tumor size in mm³, from 0 to 3000 in units of 1000, +/−SEM.

FIG. 13 is a plot showing the survival ACCX9 patient derived xenograft mice treated with vehicle, 50 mg/kg/day MBZ and 200 mg/kg/day MBZ. The X-axis shows day of treatment from, the Y-axis shows percent survival.

FIG. 14A is a plot showing the effect of the vehicle alone control on tumor size in ACCX5M1 patient derived xenograft mice. The X-axis shows day of treatment, while the Y-axis in shows tumor size in mm³.

FIG. 14B is a plot showing the effect of the 50 mg/kg/day of mebendazole on tumor size in ACCX5M1 patient derived xenograft mice. The X-axis shows day of treatment, while the Y-axis in shows tumor size in mm³.

FIG. 14C is a plot showing the effect of the 200 mg/kg/day of mebendazole on tumor size in ACCX5M1 patient derived xenograft mice. The X-axis shows day of treatment, while the Y-axis in shows tumor size in mm³.

FIG. 14D is a plot showing the effect on mean tumor size for the vehicle treated mice (circles), 50 mg/kg/day MBZ treated mice (squares) and 200 mg/kg/day MBZ treated mice (triangles) with ACCX5M1 patient derived xenograft tumors. The X-axis shows day of treatment, while the Y-axis in shows tumor size in mm³, +/−SEM.

FIG. 15 is a plot showing the survival of ACCX5M1 patient derived xenograft mice treated with vehicle, 50 mg/kg/day MBZ and 200 mg/kg/day MBZ. The X-axis shows day of treatment, the Y-axis shows percent survival.

FIG. 16A-B are each a series of three plots showing MBZ activity in adenoid cystic carcinoma (ACC) PDX tumor models. The activity of MBZ was tested in several ACC PDX tumor models.

FIG. 17A-B are each a series of three plots showing an assessment of Myb protein levels by immunohistochemistry (IHC) in adenoid cystic carcinoma (ACC) PDX tumor models.

FIG. 18A-B is a pair of graphs showing an ex vivo 3D assay utilizing primary cells isolated from two patient derived xenograft models of gastric carcinoma. In both cases, the patient derived xenografts show a significant decrease in viability following treatment with MBZ.

FIG. 19A-B are a pair of pie charts showing rare and non-rare cancer diagnoses in the U.S. in 2017 (A) and the proportion of rare and non-rare cancers in the 396 known distinct cancers (B).

FIG. 20A-B are a graph (A) and a pie chart (B) showing that common cancers are frequently rare. Major types of cancer, including breast, lung, and leukemia, are composed of many forms of cancer, both rare and common, each with distinct molecular drivers that require different treatments. As more molecular data become available, additional rare forms of cancer will likely be identified.

FIG. 21 is a series of six plots showing the determination of IC₅₀ values of MBZ in rare cancer cell lines. To assess the anti-tumor effects of MBZ in vitro, cells from rare cancer cell lines were plated into 96-well plates and allowed to incubate overnight at 37° C. The cells were then exposed to increasing concentrations of MBZ for an additional 72 hours and cell viability was measured using the Cell Titer Glo® 2.0 kit. An IC₅₀ curve was generated using the graphpad PRISM software.

FIG. 22 is a pair of tables showing a summary of IC₅₀ values of MBZ in rare cancer cell lines. To assess the anti-tumor effects of MBZ in vitro, cells from rare cancer cell lines were plated into 96-well plates and allowed to incubate overnight at 37° C. The cells were then exposed to increasing concentrations of MBZ for an additional 72 hours and cell viability was measured using the Cell Titer Glo® 2.0 kit. An IC₅₀ curve was generated using the graphpad PRISM software.

### DETAILED DESCRIPTION

The present invention is related to the finding that the anti-helminthic drug mebendazole (MBZ) can affect several relevant molecular pathways in tumor growth and metastasis. MBZ was first released in 1971, and is generally prescribed as a treatment for parasitic worm infections, including pinworm, hookworm and giardia. MBZ is thought to work as an antiparasitic by interfering with parasite carbohydrate metabolism and inhibiting polymerization of microtubules. MBZ binds to the tubulin subunits in the epithelium of the parasite, preventing polymerization of the tubulin, causing ultrastructural changes and preventing parasitic growth. Benzimidazoles such as mebendazole are structurally similar to nucleotides, allowing them to interact with a range of biomarkers and resulting in a variety of mechanisms of action in addition to preventing tubulin polymerization. For example, MBZ is capable of targeting the MYB proto-oncogene for degradation via the proteasome. MBZ has also been shown to have pro-apoptotic activity. As such, MBZ represents a novel therapeutic agent for the treatment of cancers, and one whose safety in humans as an anti-helminthic is well characterized.

While mebendazole (MBZ) is an anti-helminthic drug commonly used to treat a range of parasitic worm infections, data from preclinical in silico, in vitro, in vivo, and from human clinical studies that suggests MBZ could be a potential treatment for certain cancers. MBZ is thought to exert its anti-parasitic and anti-cancer effect at least in part through interaction with the colchicine-binding domain of tubulin preventing the polymerization of tubulin. Recently, MBZ has been shown to inhibit the growth of acute myeloid leukemia cells both in vitro and in vivo. Treatment with MBZ also results in the proteosomal degradation of the proto-oncogene transcription factor MYB, inhibiting colony-formation by AML cells. Based on this biology, rare cancer tumor cell lines including neuroblastoma, rhabdomyosarcoma and Ewing sarcoma were analyzed for sensitivity to MBZ treatment in vitro, as well as adenoid cystic carcinoma in vivo.

Cancer is a proliferative disease of a subject's own cells. In cancer, malignant cells in a subject overcome normal constraints on cellular proliferation and multiply unchecked. Because cancer is a disease of a subject's own cells the therapeutic window for treating cancer, i.e. killing only cancer cells and not healthy cells, is correspondingly narrow. Even a well-characterized and common cancer such as breast cancer, for which a variety of treatments is available, has a five-year relapse rate of around 7 to 13 percent. There thus exists an unmet need in the field for additional cancer therapies. This need is particularly acute with respect to rare cancers. Rare cancers are defined by the National Cancer Institute as cancers that occur in less than 15 cases per 100,000 people per year. The American Cancer Society's (ACS) metric for rare cancers is less than 6 per 100,000 incidence. Using this metric, of 396 known distinct cancers, 374 are rare forms. Cumulatively, estimates for the number of rare cancers diagnosed in adults range between 22% and 42% of all diagnosed cancers. In 2017, by the conservative ACS metric, there were over 500,000 rare cancer diagnoses in the United States alone. Extrapolating from U.S. metrics, the U.S., E.U. and China alone likely had over 3.2 million rare cancer diagnoses in 2017. However, because each cancer is unique, recently developed targeted cancer therapies, such as immune and antibody based therapies or chemotherapies that target particular pathways specific to certain cancers may not work or have not been investigated in rare cancers. Depending upon the definition used, as many as half of all cancers diagnosed are considered rare cancers. The disclosure provides methods for using an additional therapeutic agent, mebendazole (MBZ), for use in the cancer treatment arsenal.

### Mebendazole

The disclosure provides the use of mebendazole, or a pharmaceutically acceptable salt, polymorph or solvate thereof in the treatment of cancer in a subject, or for the preparation of a therapeutically effective composition useful for the treatment of such a cancer. In some embodiments, mebendazole may be used as a monotherapy. Alternatively, in some embodiments, mebendazole may be combined with additional cancer therapies or treatments. The disclosure provides a method of treating a cancer in a subject in need thereof, comprising administering to the subject a therapeutically effective amount of a composition comprising methyl N-(6-benzoyl-1H-benzimidazol-2-yl)carbamate (mebendazole, or MBZ). In some embodiments, mebendazole is also known as 5-Benzoyl-2-benzimidazolecarbamic acid methyl ester, Mebendazol, Methyl N-(5-benzoyl-1H-benzimidazol-2-yl)carbamate. Mebendazole comprises a structure of:

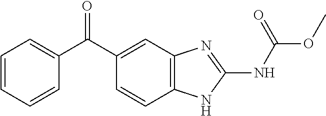 [SMILES: COC(=O)Nc1nc2cc(C(=O)c3ccccc3)ccc2[nH]1]

In some embodiments of the mebendazole of the disclosure, the mebendazole comprises a salt. Exemplary, but non-limiting examples of the mebendazole salts include a mebendazole hydrochloride salt ((5-benzoyl-1H-benzimidazole-2-yl)-carbamic acid methyl ester hydrochloride, MBZ.HCl), a mebendazole hydrobromide salt, a mebendazole maleate salt, a mebendazole-gutarate salt and a mebendazole monomethyl oxalate salt. The mebendazole may comprise a co-crystal, such as an acid co-crystal. For example, the mebendazole is a mebendazole-glutaric acid co-crystal.

In some embodiments, the mebendazole of the disclosure comprises a solvate. In some embodiments, the solvate comprises one or more water molecules (a hydrate). In some embodiments, the mebendazole comprises a monohydrate. An exemplary monohydrate comprises mebendazole mesylate monohydrate.

In some embodiments, the mebendazole of the disclosure comprises a crystal. In some embodiments, the mebendazole crystal comprises different polymorphs. In some embodiments, the composition comprising mebendazole comprises a crystal polymorph A of mebendazole, a crystal polymorph B of mebendazole, a crystal polymorph C of mebendazole or a combination thereof. In some embodiments, the mebendazole comprises crystal polymorph A. In some embodiments, the mebendazole comprises crystal polymorph B. In some embodiments, the mebendazole comprises crystal polymorph C. In some embodiments, the mebendazole comprises a combination of one or of the crystal polymorphs of mebendazole. Crystal polymorphs of mebendazole are described in Chinese patent CN85103977B, the contents of which are incorporated herein by reference in their entirety. The three crystal polymorphs of mebendazole can be characterized by Fourier transform infrared (FTIR), differential scanning calorimetry (DSC), dissolution, solubility and X-Ray diffraction pattern. In some embodiments of the therapeutically effective composition comprising mebendazole, the solubility of the three polymorphs differs. In some embodiments of the therapeutically effective composition comprising mebendazole, mebendazole crystal polymorph C is the most soluble of the three crystal polymorphs of mebendazole.

Administering a composition comprising mebendazole, or a pharmaceutically acceptable salt, polymorph or solvate thereof, to a cell or a subject in need thereof results in modulation (i.e., stimulation or inhibition) of an activity of an intracellular target (e.g., substrate). In some embodiments, several intracellular targets can be modulated with the composition comprising mebendazole.

Without wishing to be bound by any particular theory or limit the mechanisms or biological pathways through which mebendazole may act, set forth are some ways in which mebendazole can affect the cancer cells of a subject.

In some embodiments, the therapeutically effective composition that comprises mebendazole interferes with the carbohydrate metabolism of the cancer cells of a subject. Exemplary effects of inhibiting carbohydrate metabolism comprise causing glycogen depletion, inhibiting glucose uptake, and/or disrupting enzymes involved in carbohydrate metabolism. In some embodiments, interfering with carbohydrate metabolism causes the death of the cell.

In some embodiments, mebendazole binds to tubulin subunits in the cancer cells of the subject preventing microtubule polymerization. In some embodiments, preventing microtubule polymerization disrupts microtubule structure, thus interrupting the metastatic cascade. Mebendazole differs from conventional cytotoxic microtubule targeted drugs in that it binds to the colchicine binding site. Colchicine binds to beta-tubulin, inducing a conformational change to produce a curved tubulin dimer, which inhibits microtubule assembly. In contrast, classical chemotherapeutic agents such as paclitaxel bind to the taxol binding site, while vinblastine binds to the vinblastine binding site.

In some embodiments, mebendazole targets the MYB proto-oncogene (MYB) for degradation by the proteasome in the cancer cells of a subject. MYB is a member of the myeloblastosis family of transcription factors. MYB is a key regulator of stem and progenitor cells in the bone marrow, colonic crypts and neurogenic region of the adult brain. MYB is thought to be an oncogene that is involved in some human leukemias and adenoid cystic carcinoma and is activated either through overexpression or mis-expression in some colon and breast cancers. In some embodiments, genetic mutations the MYB coding sequence, in the MYB non-coding sequence or a combination thereof result in the overexpression or mis-expression of MYB. In some embodiments, the MYB non-coding sequence comprises a 5′ untranslated region (UTR), a 3′ UTR, an enhancer, a promoter, an intron, an insulator, a DNA structural element or a combination thereof. In some embodiments, reducing the level of MYB activity alleviates a sign or a symptom of the cancer. In some embodiments, the level of MYB is reduced by targeting MYB protein for degradation by the proteasome. In some embodiments, administering a therapeutically effective amount of a composition comprising mebendazole targets MYB for degradation by the proteasome.

As used herein, the “proteasome” refers to a protein complex that degrades proteins in a cell by proteolysis. In some embodiments, the proteins to be degraded are unneeded or damaged. In some embodiments, the proteins to be degraded are targeted for degradation by being tagged with a small protein called ubiquitin. In some embodiments, the ubiquitin tag is a polyubiquitin chain. In some embodiments, the polyubiquitin chain allows the proteasome to bind to and degrade the targeted protein.

In some embodiments, mebendazole elicits mitochondrial cytochrome c release followed by apoptosis in the cancer cells of a subject. During apoptosis, the mitochondria release of cytochrome c into the cytosol, which triggers a cascade of caspase activation, leading to cell death. In some embodiments, mebendazole's cytotoxic function is mediated through phosphorylation of Bcl-2 (BCL2, apoptosis regulator), preventing Bcl-2 interaction with the proapoptotic BCL2 associated X, apoptosis regulator (Bax) protein and promoting apoptosis. Bcl-2 is an integral outer mitochondrial membrane protein that blocks apoptotic death in some cells. Bax is a member of the Bcl-2 protein family and a proapoptotic protein. Upon triggering of apoptosis, Bax and the related protein BCL2 antagonist/killer 1 (Bak) form oligomers in the outer mitochondrial membrane, allowing contents from the mitochondrial intermembrane space to translocate to the cytosol, leading to apoptosis.

Contacting a cell with a composition comprising mebendazole, or a pharmaceutically acceptable salt, polymorph or solvate thereof or nanoparticle formulation thereof can induce or activate cell death preferentially in cancer cells. Administering to a subject in need thereof a composition comprising mebendazole, or a pharmaceutically acceptable salt, polymorph or solvate thereof or nanoparticle formulation thereof can induce or activate cell death preferentially in cancer cells. Contacting a cell with mebendazole, or a pharmaceutically acceptable salt, polymorph or solvate thereof or nanoparticle formulation thereof can induce cell death selectively in one or more cells affected by a cell proliferative disorder. Preferably, administering to a subject in need thereof a composition comprising mebendazole, or a pharmaceutically acceptable salt, polymorph or solvate thereof or nanoparticle formulation thereof induces cell death preferentially in one or more cells affected by a cell proliferative disorder. Preferably, the overall toxicity of the therapeutic amount of the composition comprising is tolerable to normal cells.

In some embodiments, mebendazole can be formulated as a salt. Exemplary but non-limiting examples of mebendazole salts comprise mebendazole hydrochloride salt ((5-benzoyl-1H-benzimidazole-2-yl)-carbamic acid methyl ester hydrochloride, MBZ.HCl), a mebendazole hydrobromide salt, a mebendazole maleate salt, a mebendazole glutarate salt, a mebendazole monomethyl oxalate salt or a mebendazole mesylate monohydrate.

Additionally, in some embodiments of the mebendazole of the present disclosure, a salt of mebendazole can exist in either hydrated or unhydrated (the anhydrous) form or as solvates with other solvent molecules. Non-limiting examples of hydrates include monohydrates, dihydrates, and so forth. Non-limiting examples of solvates include ethanol solvates, acetone solvates, and forth.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

It should be understood that all references to pharmaceutically acceptable salts include solvent addition forms (solvates) or crystal forms (polymorphs) as defined herein, of the same salt.

In some embodiments of the methods of the disclosure, the composition comprising mebendazole further comprises a nanoparticle. In some embodiments, the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a diblock copolymer, a polymeric micelle or a hyperbranched macromolecule. In some embodiments, the polymer-based nanoparticle comprises a multiblock copolymer a diblock copolymer. In some embodiments, the polymer-based nanoparticle is pH responsive. In some embodiments, the polymer-based nanoparticle further comprises a buffering component.

In some embodiments, the composition comprising mebendazole further comprises a nanoparticle. In some embodiments, the nanoparticle comprises a liposome. Liposomes are spherical vesicles having at least one lipid bilayer, and in some embodiments, an aqueous core. In some embodiments, the lipid bilayer of the liposome may comprise phosphilipids. An exemplary but non-limiting example of a phospholipid is phosphatidylcholine, but the lipid bilayer may comprise additional lipids, such as phosphatydilethanoamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Published Application 20090023673. In other embodiments, the one or more lipids are one or more cationic lipids. One skilled in the art will recognize which liposomes are appropriate for mebendazole encapsulation.

In some embodiments, the nanoparticle comprises a micelle. A micelle is an aggregate if surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In some embodiments, the nanoparticle comprises a polymer based nanoparticle. In some embodiments, the polymers comprise a multiblock copolymer, a diblock copolymer, a polymeric micelle or a hyperbranched macromolecule. In some embodiments, the particle comprises one or more cationic polymers. In some embodiments, the cationic polymer is chitosan, protamine, polylysine, polyhistidine, polyarginine or poly(ethylene)imine. In other embodiments, the one or more polymers contain the buffering component, degradable component, hydrophilic component, cleavable bond component or some combination thereof.

In some embodiments, the nanoparticles or some portion thereof are degradable. In other embodiments, the lipids and/or polymers of the nanoparticles are degradable.

In some embodiments, any of these nanoparticles can comprise a buffering component. In other embodiments, any of the nanoparticles can comprise a buffering component and a degradable component. In still other embodiments, any of the nanoparticles can comprise a buffering component and a hydrophilic component. In yet other embodiments, any of the nanoparticles can comprise a buffering component and a cleavable bond component. In yet other embodiments, any of the nanoparticles can comprise a buffering component, a degradable component and a hydrophilic component. In still other embodiments, any of the nanoparticles can comprise a buffering component, a degradable component and a cleavable bond component. In further embodiments, any of the nanoparticles can comprise a buffering component, a hydrophilic component and a cleavable bond component. In yet another embodiment, any of the nanoparticles can comprise a buffering component, a degradable component, a hydrophilic component and a cleavable bond component. In some embodiments, the particle is composed of one or more polymers that contain any of the aforementioned combinations of components.

In some embodiments, the nanoparticle further comprises mebendazole. In some embodiments, the mebendazole is on the surface and/or within the nanoparticle. In other embodiments, the mebendazole is conjugated to, complexed to or encapsulated within the nanoparticle. In further embodiments, the mebendazole is conjugated to, complexed to or encapsulated by the one or more lipids or polymers of the nanoparticles. In some embodiments, the conjugation is covalent. In some embodiments, the mebendazole is intercalated within the lipids or polymers of the nanoparticle.

In some embodiments, the nanoparticle further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment. In some embodiments, the targeting agent binds to the surface of a cell of the cancer of the subject. In some embodiments, the targeting agent is on the surface and/or within the nanoparticle.

In certain embodiments, the targeting agent comprises hyaluronic acid (HA). HA binds to CD44, a transmembrane peptidoglycan expressed on the surface of many types of cancer cells. CD44 integrates cellular environmental cues with growth factors and cytokine signals, and plays a role in the progression of many cancers. Targeting of CD44+ cells by HA nanoparticles thus provides superior delivery and specificity of the compositions of the disclosure to cancer cells.

In some embodiments, the nanoparticle further comprises a blending polymer. In some embodiments, the blending polymer is a copolymer comprising a degradable component and hydrophilic component. In some embodiments, the degradable component of the blending polymer is a polyester, poly(ortho ester), poly(ethylene imine), poly(caprolactone), polyanhydride, poly(acrylic acid), polyglycolide or poly(urethane). In some embodiments, the degradable component of the blending polymer is poly(lactic acid) (PLA) or poly(lactic-co-glycolic acid) (PLGA). In some embodiments, the hydrophilic component of the blending polymer is a polyalkylene glycol or a polyalkylene oxide. In some embodiments, the polyalkylene glycol is polyethylene glycol (PEG). In other embodiments, the polyalkylene oxide is polyethylene oxide (PEO).

In some embodiments, the one or more polymers comprise a polyester, poly(ortho ester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In some embodiments, the one or more polymers comprise polyalkylene glycol or a polyalkylene oxide. In some embodiments, the polyalkylene glycol is polyethylene glycol (PEG) or the polyalkylene oxide is polyethylene oxide (PEO).

In some embodiments, the nanoparticle comprises a nanocrystal. Exemplary nanocrystals are crystalline particles with at least one dimension of less than 1000 nanometers, preferably of less than 100 nanometers.

In some embodiments, the nanoparticle has an average characteristic dimension of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In other embodiments, the nanoparticle has an average characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In further embodiments, the nanoparticle has an average characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

Sterile injectable solutions comprising a nanoparticle of the disclosure can be prepared by incorporating the mebendazole in the nanoparticles in the required amount in an appropriate solvent with one or a combination of ingredients enumerated herein, as required, followed by filtered sterilization. Alternatively, or in addition, sterilization can be achieved through other means such as radiation or gas. Generally, dispersions are prepared by incorporating the nanoparticles into a sterile vehicle that contains a basic dispersion medium and the required other ingredients from those enumerated above. In the case of sterile powders for the preparation of sterile injectable solutions, methods of preparation are vacuum drying and freeze-drying that yields a powder of mebendazole nanoparticles plus any additional desired ingredient from a previously sterile-filtered solution thereof.

Oral compositions comprising the mebendazole nanoparticles of the disclosure generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the nanoparticles in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as sodium starch glycolate, starch or lactose, a diluent such as microcrystalline cellulose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the mebendazole nanoparticle is delivered in the form of an aerosol spray from pressured container or dispenser, which contains a suitable propellant, e.g., a gas such as carbon dioxide, or a nebulizer.

Systemic administration of a nanoparticle of the disclosure can also be by transmucosal, subcutaneous, intramuscular or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the formulation into salves, gels, or creams as generally known in the art.

The mebendazole nanoparticles can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, for example those embodiments wherein the composition comprising mebendazole is administered with one or more additional cancer therapies, the nanoparticle comprises mebendazole and one or more additional therapeutic or chemotherapeutic agents. For example, the nanoparticle comprises mebendazole and Paclitaxel, Docetaxel, Vinblastine, Vincristine, Cisplatin, Carboplatin, Oxaliplatin, Doxorubicin, Etoposide, Imatinib, Gemcitabine, Vinorelbine, Ifosamide, Abemaciclib, Sorafenib, Irinotecan, 5-Fluorouracil, Dacarbazine, Trabectedin, Temozolomide or Cyclophosphamide. In some embodiments, the nanoparticle comprises a synergistic combination of mebendazole and one or more therapeutic or chemotherapeutic agents.

### Cancers

The methods of the disclosure are not intended to be limited to any particular sort of cancer. Indeed, given the effects of mebendazole on multiple cellular pathways, treatment methods comprising administering a therapeutically effective amount of a composition comprising mebendazole to a subject with a cancer are anticipated to be effective on a wide array of cancers in which these cellular pathways have been implicated.

The effectiveness of mebendazole in the treatment of particular cancer or type of cancer can be assayed using a variety of in vitro and in vivo models, as well be set forth in greater detail in the Examples. One approach to determining the effectiveness of mebendazole in the treatment of cancer comprises administering increasing concentrations of mebendazole to an exemplary cancer cell line, and determining the IC₅₀ value. As used herein, the term “IC₅₀ value” refers to the concentration of a compound wherein the response to that compound is reduced by half. The IC₅₀ is thus a measure of the effectiveness of a compound in inhibiting a biological process. In this model, cancerous cell lines indicative of the various cancers of the disclosures are cultured using standard techniques, treated with mebendazole, and the IC₅₀ value is calculated after 24, 48 or 72 hours to determine the effectiveness of mebendazole in killing the cancer cells.

Alternatively, or in addition, the effectiveness of mebendazole can be assayed in vivo using a standard cell line xenograft or a patient derived xenograft mouse model. In patient derived xenograft mice, cancerous cells isolated from a cancer patient or from a cancer cell line are implanted into an immunodeficient mouse, and allowed to form tumors. The mice are then administered mebendazole, and the effect on tumor size and mouse survival is assayed. As xenograft cancers can be implanted from a variety of cell lines and patient sources, the effectiveness of mebendazole treatment on multiple cancers can be assayed in this manner.

In some embodiments, the cancer treated by a composition comprising a therapeutically effective amount of mebendazole comprises a commonly occurring cancer. In some embodiments, the cancer treated by a composition comprising a therapeutically effective amount of mebendazole comprises a colorectal cancer, a gastric cancer, a brain cancer, a colon cancer, a breast cancer, a liver cancer, a lung cancer, a pancreatic cancer or a renal cancer.

In some embodiments, the cancer treated by a composition comprising a therapeutically effective amount of mebendazole comprises a colon cancer. Colon cancers are cancers of the large intestine, or colon, which is the final part of the digestive tract. In some embodiments, colon cancers begin as small noncancerous clumps of cells called adenomatous polyps. These polyps typically form on the inner walls of the large intestine. In some embodiments, colon cancer cells can metastasize throughout the body. Typically, colon cancer occurs in people who are over 50 years of age. Risk factors include obesity, diet, smoking, and genetic factors.

In some embodiments, the cancer treated by a composition comprising a therapeutically effective amount of mebendazole comprises a colorectal cancer. Colorectal cancers comprise cancers of the colon or rectum. The rectum is the passageway that connects the colon to the anus. In certain embodiments, the colorectal cancer comprises a colorectal carcinoma.

In some embodiments, the cancer treated by a composition comprising a therapeutically effective amount of mebendazole comprises a gastric cancer. Gastric cancers comprise cancers which form from the cells of the lining of the stomach.

In some embodiments, the cancer treated by a composition comprising a therapeutically effective amount of mebendazole comprises a brain cancer. Brain cancers comprise cancers that form from cells of the brain. Alternatively, or in addition, brain cancers comprise cancers located in, on or in close proximity to the brain. There are many types of brain cancers. Exemplary but non-limiting brain cancers comprise gliomas, neuromas, astrocytomas, glioblastomas, craniopharyngiomas and medulloblastomas.

In some embodiments, the cancer treated by a composition comprising a therapeutically effective amount of mebendazole comprises a liver cancer. Liver cancers comprise cancers that form from cells of the liver. Exemplary but non-limiting liver cancers include hepatocellular carcinoma, cholangiocarcinoma and hepatoblastoma. In some embodiments, the liver cancer comprises a hepatocellular carcinoma. In some embodiments, the hepatocellular carcinoma occurs in a patient with chronic liver disease and cirrhosis. In some embodiments, the hepatocellular carcinoma forms from hepatic stem cells. In some embodiments, the liver cancer comprises a cholangiocarcinoma. In some embodiments, the cholangiocarcinoma forms in the bile ducts just outside the liver. In some embodiments, the cholangiocarcinoma is intrahepatic, extrahepatic (i.e., perihilar) or a distal extrahepatic cholangiocarcinoma. In some embodiments, the liver cancer comprises a hepatoblastoma. In some embodiments, the hepatoblastoma occurs in a child or an infant. In some embodiments, the hepatoblastoma originates from immature liver precursor cells. In some embodiments, the hepatoblastoma originates from pluripotent stem cells. In some embodiments, risk factors for liver cancer include obesity, diet, smoking, and genetic factors.

In some embodiments, the cancer treated by a composition comprising a therapeutically effective amount of mebendazole comprises a lung cancer. In some embodiments, the lung cancer is a small cell lung cancer. In some embodiments, the small cell lung cancer is a small cell carcinoma (oat cell cancer) or a combined small cell carcinoma. In some embodiments, the small cell carcinoma comprises a neuroendocrine subtype of lung cancer that likely arises from neuroendocrine cells in the lung. Risk factors include asbestos exposure and smoking. In some embodiments, the lung cancer is a non-small cell lung cancer. In some embodiments, the non-small cell lung cancer is a non-small cell lung carcinoma. In some embodiments, the non-small cell lung carcinoma is an epithelial lung cancer other than small cell lung carcinoma. In some embodiments the non-small cell lung cancer is an adenocarcinoma, a squamous cell (epidermoid) carcinoma, an adenosquamous carcinoma or a sarcomatoid carcinoma. Squamous cells are flat cells that line the insides of the airways in the lungs.

In some embodiments, the cancer treated by a composition comprising a therapeutically effective amount of mebendazole comprises a pancreatic cancer. Pancreatic cancers typically arise from the cells in the pancreas, a glandular organ behind the stomach. In some, more frequent, embodiments, the pancreatic cancer is a pancreatic adenocarcinoma. Typically, pancreatic adenocarcinomas arise from the part of the pancreas which makes digestive enzymes. In some embodiments, the pancreatic cancer is a neuroendocrine tumor, which arises from the hormone producing cells of the pancreas.

In some embodiments, the cancer treated by a composition comprising a therapeutically effective amount of mebendazole comprises a renal cancer. Renal cancers are cancers that arise from cells of the kidney. In some embodiments, the renal cancer first appears in the tubules of the kidney. In some embodiments, the renal cancer is an adult cancer. In some embodiments, the renal cancer is a pediatric cancer. In some embodiments, the renal cancer is a renal cell carcinoma, an inherited papillary renal cell carcinoma, a urothelial cell carcinoma of the renal pelvis, a squamous cell carcinoma, ajuxtaglomerular cell tumor (reninoma), an angiomyolipoma, a renal oncocytoma, a Bellini duct carcinoma, a clear-cell sarcoma of the kidney, a mesoblastic nephroma, a Wilms' tumor (usually diagnosed in children under 5 years of age) or a mixed epithelial stromal tumor.

In some embodiments, the cancer treated by a composition comprising a therapeutically effective amount of mebendazole comprises a rare cancer. Rare cancers are cancer that affect a very small number of people. Rare cancers are defined by the National Cancer Institute as cancers that occur in less than 15 cases per 100,000 people per year. Alternatively, a consortium from the European Union (RARECARE) defines rare cancers as those with few than 6 cases per 100,000 people per year. Alternatively, or in addition, a cancer might be considered rare if it starts in an unusual place in the body, or if it is of an unusual type and needs special treatment.

In some embodiments, the rare cancer treated by a composition comprising a therapeutically effective amount of mebendazole comprises a blastoma. Blastomas are cancers that arise from precursor cells, also known as blast cells. As used herein, the term “blast cells” refers to immature, not fully differentiated cells in the body. Exemplary blast cells comprise bone marrow blast cells. In some embodiments, the subject with a blastoma is a child. In some embodiments, the subject with a blastoma is an adult. Exemplary but not limiting blastomas comprise nephroblastoma, medulloblastoma, retinoblastoma and neuroblastoma.

In some embodiments, the blastoma is a neuroblastoma. In some embodiments, neuroblastomas are cancers that begin in certain forms of nerve cells typically found in an embryo or fetus. In some embodiments, the nerve cells that give rise to the neuroblastoma are neuroblasts. Neuroblastomas occur most frequently in infants and young children, and are found only rarely in subjects older than ten years of age. In some embodiments, the neuroblastoma starts in the adrenal gland. In some embodiments, the neuroblastoma starts in the sympathetic nerve ganglia in the abdomen. In some embodiments, the neuroblastoma starts in the sympathetic nerve ganglia near the spine in the chest, neck or pelvis. In some embodiments, the neuroblastoma is a ganglioneuroblastoma. In some embodiments, the ganglioneuroblastoma comprises both malignant and benign components.

In some embodiments, the blastoma is a glioblastoma, also known as glioblastoma multiforme. In some embodiments, the glioblastoma forms from astrocytes. Astrocytes, also known as astroglia, are star shaped glial cells in the brain and spinal chord. Astrocytes perform a variety of functions in the nervous system, including but not limited to axon guidance, synaptic support, and control of the blood flow. In some embodiments, the glioblastoma is an astrocytoma. In some embodiments, the glioblastoma is a primary glioblastoma. Primary glioblastomas develop rapidly de novo without clinical or histological evidence of a less malignant precursor lesion. Primary glioblastomas frequently occur in the elderly. In some embodiments, the glioblastoma is a secondary glioblastoma. In some embodiments, the secondary glioblastoma progresses from a low grade diffuse astrocytoma or an anaplastic astrocytoma. Secondary glioblastomas frequently occur in younger patients and have a lesser degree of necrosis. Secondary glioblastomas are frequently located in the frontal lobe. In some embodiments, the glioblastoma is an astrocytoma. In some embodiments, the glioblastoma forms in the brain or in the nerve chord.

In some embodiments, the rare cancer treated by a composition comprising a therapeutically effective amount of mebendazole comprises a carcinoma. Carcinomas are cancers that arise from epithelial tissues of the skin and mucous membranes or linings of internal organs, glands, the bladder and nerves and so forth. In early stages, a carcinoma will be confined to the layer of the tissue in which it started. In later stages, the carcinoma spreads to surrounding tissues, and metastasizes throughout the body. In some embodiments, rare carcinomas to be treated by the methods of the disclosure comprise acinic cell carcinoma, adenoid cystic carcinoma (ACC), adrenocortical carcinoma, adenocarcinoma of the appendix, ameloblastic carcinoma, basal cell carcinoma (infundibulocystic), basal cell carcinoma (multiple), carcinoma of the vocal tract, childhood carcinoma of unknown primary site, childhood hepatocellular carcinoma, choriocarcinoma, choroid plexus carcinoma, chromophil renal cell carcinoma, clear cell renal cell carcinoma, collecting duct carcinoma, eccrine mucinous carcinoma, eccrine porocarcinoma, embryonal carcinoma, epithelial-myoepithelial carcinoma, fibrolamelar carcinoma, glassy cell carcinoma of the cervix, hereditary renal cell carcinoma, intrahepatic cholangiocarcinoma, keratosis palmoplantaris adenocarcinoma of the colon, krukenberg carcinoma, lung adenocarcinoma, Merkel cell carcinoma, metaplastic carcinoma of the breast, mucoepidermoid carcinoma, myoepithelia carcinoma, nasopharyngeal carcinoma, nevoid basal cell carcinoma syndrome, ovarian small cell carcinoma, pancreatic carcinoma, papillary cystadenocarcinoma, papillary renal cell carcinoma, papillary thyroid carcinoma, parathyroid carcinoma, polymorphous low grade adenocarcinoma, familial renal carcinoma, renal cell carcinoma 4, secretory breast carcinoma, sinonasale undifferentiated carcinoma, small cell carcinoma of the bladder, rare adenocarcinoma of the breast, transitional cell carcinoma and urachal adenocarcinoma. Alternatively, or in addition, in some embodiments, rare carcinomas to be treated by the methods of the disclosure comprise adenoid cystic carcinoma (ACC), uterine serous carcinoma, adrenocortical carcinoma, gastric carcinoma, ovarian carcinoma, thymic carcinoma, cholangiocarcinoma, colorectal carcinoma and esophageal carcinoma.

In some embodiments, the rare carcinoma is an adenoid cystic carcinoma (ACC). ACC is a rare form of adenocarcinoma, a cancer that begins in glandular tissues. ACC is found mainly in the head and neck, but can occasionally occur in other locations, such as the uterus, the trachea, the lacrimal gland, breast skin or vulva. As such, in some embodiments ACC can comprise a salivary gland cell, a trachea cell, a lacrimal gland cell, a breast cell, a skin cell or a vulval cell. In some common embodiments, ACC occurs in the salivary glands scattered throughout the upper aerodigestive tract. In some embodiments, ACC spreads along the nerves or through the bloodstream. In some embodiments, ACC will spread to the lymph nodes, the lungs or a combination thereof. In some embodiments, ACC is classified based on histological variations in the tumor. Exemplary classifications include cylindroma, cribiform or solid.

In some embodiments, the rare carcinoma is a uterine serous carcinoma. Also called papillary serous carcinoma, uterine papillary serous carcinoma (UPSC), endometrial type 2 tumor, uterine serous carcinoma is a rare form of endometrial cancer that typically arises in postmenopausal women. In some embodiments, uterine serous carcinoma is associated with mutations in the p53 tumor suppressor. In some embodiments, uterine serous carcinoma may spread throughout the abdomen. In some embodiments, the uterine serous carcinoma may be a superficial endometrial tumor with extensive peritoneal disease.

In some embodiments, the rare carcinoma is an adrenocortical carcinoma. In adrenocortical carcinoma, the cancer forms on the cortex (the outer layer) of the adrenal gland. The adrenal gland sits on top of the kidney and the cortex makes hormones. In some embodiments, these hormones include hormones involved in water and salt homeostasis and blood pressure. In some embodiments, the adrenocortical carcinoma comprises a tumor that increases the amount of hormones produced by the adrenal cortex. In some embodiments, genetic disorders such as Li-Fraumeni syndrome, Beckwith-Wiedemann syndrome and Carney complex increase the risk of a subject with the disorder of developing adrenocortical carcinoma.

In some embodiments, the rare carcinoma is an ovarian carcinoma. In some embodiments, an ovarian carcinoma is a carcinoma that forms on or in an ovary of a subject. In some embodiments, the carcinoma forms on the fallopian tubes. In some, more frequent, embodiments, the ovarian carcinoma is an epithelial ovarian carcinoma. In some embodiments, the ovarian carcinoma is a papillary serous carcinoma. In some embodiments, the ovarian carcinoma comprises a germ cell carcinoma. The most common types of germ cell carcinomas are teratomas, dysgermimomas and endodermal sinus tumors. In some embodiments, the ovarian carcinoma comprises a stromal carcinoma. An ovarian stromal carcinoma develops from the connective tissue cells that hold the ovary together and produce female hormones such as estrogen and progesterone. Ovarian stromal carcinomas comprise granulosa cell tumors and Steroli-Leydig cell tumors. In some embodiments, the ovarian carcinoma comprises a small cell carcinoma of the ovary. Small cell carcinomas of the ovary are rare, highly malignant, and typically affect young women. Small cell carcinomas of the ovary comprise pulmonary, neuroendocrine and hypercalcemic small cell carcinomas of the ovary.

In some embodiments, the rare carcinoma is a gastric carcinoma. In some embodiments, the gastric carcinoma comprises an adenocarcinoma. Gastric adenocarcinomas develop from the cells that form the innermost lining of the stomach, also known as mucosa. Rarely, in some embodiments, gastric cancers arise from cells in the walls of the stomach called interstitial cells of Cajal (GIST tumors, also known as a gastrointestinal stromal tumor). In some embodiments, gastric cancers comprise carcinoid tumors. Carcinoid tumors arise from hormone producing cells of the stomach. In some rare embodiments, a squamous cell carcinoma, a small cell carcinoma or a leiomysarcoma can form from cells of the stomach. There are many risk factors for gastric cancers. Exemplary risk factors comprise Helicobacter pylori infection, diet, Epstein-Barr virus infection, chronic inflammation, smoking and hereditary factors.

In some embodiments, the rare cancer treated by a composition comprising a therapeutically effective amount of mebendazole comprises a cholangiocarcinoma. Cholangiocarcinomas are tumors arising in the cells of the connective tissues of the bile ducts. In some embodiments, liver disease or colitis increases the risk of a subject developing cholangiocarcinoma. In some embodiments, the cholangiocarcinoma comprises an intrahepatic bile duct cancer. Intrahepatic bile duct cancers form in the bile ducts inside the liver. In some embodiments, the cholangiocarcinoma comprises an extrahepatic bile duct cancer. In some embodiments, the extrahepatic bile duct cancers form in the perihilar bile duct or the distal extrahepatic bile duct. In some embodiments, a subject with a cholangiocarcinoma produces a higher than normal level of a tumor marker such as CEA or CA 19-9 in blood, urine or tissue.

In some embodiments, the rare cancer treated by a composition comprising a therapeutically effective amount of mebendazole comprises a neuroendocrine cancer. In some embodiments, the neuroendocrine cancer comprises a thymic cancer. In some embodiments, the thymic cancer comprises a thymic carcinoma or a thymoma. Thymic carcinomas are tumors that form on the outside surface of the thymus, a gland in the upper chest. In some embodiments, the thymic carcinoma is derived from thymic epithelia cells. In some embodiments, early thymic carcinoma may be asymptomatic, so that a thymic carcinoma of a subject is only identified late in the progression of the disease. In some embodiments, the thymic carcinoma may be highly aggressive. In some embodiments, the overall 5-year survival rate for patients with thymic carcinoma is only 30-50%. In some embodiments, the thymic cancer comprises a thymoma. In some embodiments, the cells of a thymoma tend to resemble normal thymus cells, grow slowly and rarely spread beyond the thymus.

In some embodiments, the rare cancer treated by a composition comprising a therapeutically effective amount of mebendazole comprises a sarcoma. Sarcomas are rare cancers that arise from mesenchymal cells. In some embodiments, the sarcoma comprises a malignant tumor comprising cells arising from cancellous bone, cartilage, fat, muscle, vasculature or hematopoietic tissues. In some embodiments, the sarcoma comprises adenosarcoma of the uterus, alveolar soft part sarcoma, angiosarcoma of the breast, angiosarcoma of the liver, angiosarcoma of the scalp, cerebral sarcoma, chondrosarcoma, chromophil renal cell sarcoma, embryonal sarcoma, endemic Kaposi sarcoma, endometrial stromal sarcoma, Ewing's sarcoma, fibrosarcoma, gliosarcoma, Langerhans cell sarcoma, leiomyosarcoma, lymphosarcoma, malignant teratocacinosarcoma, microcystic adnexal carcinoma, myxoid liposarcoma, neurofibrosarcoma, oral squamous sarcoma, osteosarcoma, ovarian carcinosarcoma, paraganglioma and gastric stromal sarcoma, plexosarcoma, radiation induced angiosarcoma of the breast, alveolar rhabdomyosarcoma, embryonal rhabdomyosarcoma, soft tissue sarcoma, childhood soft tissue sarcoma, synovial sarcoma, undifferentiated pleiomorphic sarcoma, uterine carcinosarcoma or uterine sarcoma. Alternatively, or in addition, the sarcoma comprises an Ewing's sarcoma, a leiomyosarcoma, an angiosarcoma, or a rhabdomyosarcoma.

In some embodiments, the rare cancer treated by a composition comprising a therapeutically effective amount of mebendazole comprises an Ewing's sarcoma. Ewing's sarcoma comprises tumors of the bones, the soft tissue surrounding bones such as cartilage and nerves, or a combination thereof. Ewing's sarcoma typically affects children and young adults, although it can occur at any age. Ewing's sarcoma can occur in any bone. In some more frequent embodiments, Ewing's sarcoma begins in the leg bones, hipbones, arm bones, and bones in the chest, skull or spine. In some less common embodiments, Ewing's sarcoma occurs in the soft tissues of the arms, legs, abdomen, chest, neck, head or a combination thereof. In some embodiments of Ewing's sarcoma, there is no bone involvement. In some embodiments, treatments for Ewing's sarcoma comprise chemotherapy, surgery, or a combination thereof. In some embodiments, the chemotherapy comprises neoadjuvant chemotherapy, which may comprise vincristine, doxorubicin and cyclophosphamide with ifosfamide and etoposide. In some embodiments, Ewing's sarcoma is associated with a chromosomal translocations affecting the EWSR1 (EWS RNA binding protein 1), FLI1 (Fli-1 proto-oncogene), ERG (ERG, ETS transcription factor) and ETV1 (ETS variant 1) genes.

In some embodiments, the rare cancer treated by a composition comprising a therapeutically effective amount of mebendazole comprises a leiomyosarcoma. Leiomyosarcomas are a type of soft tissue sarcoma. In some embodiments, the leiomyosarcoma comprises a malignant tumor that arises from smooth muscle cells. Smooth muscles cells are the cells of involuntary muscles, i.e. muscles over which the brain has no voluntary control. Exemplary involuntary muscles comprise the walls of the digestive tract and muscles controlling salivary gland secretions. In some embodiments, the leiomyosarcoma grows and spreads into surrounding tissues. In some embodiments, the leiomyosarcoma spreads to distant sites of the body via the bloodstream or lymphatic system, or both. In some embodiments, a leiomyosarcoma can form almost anywhere where there are blood vessels, such as the heart, liver, pancreas, genitourinary and gastrointestinal tract, the space behind the abdominal cavity (retroperitoneum), the uterus or skin. In some of the more common embodiments, the leiomyosarcoma forms in the uterus. Symptoms, diagnosis and treatment of leiomyosarcomas varies depending on the location and stage of the cancer.

In some embodiments, the rare cancer treated by a composition comprising a therapeutically effective amount of mebendazole comprises an angiosarcoma. Angiosarcomas are sarcomas arising from cells of the inner lining of the blood vessels. Angiosarcomas can occur in any area of the body. In some embodiments, the angiosarcoma occurs in the skin, breast, liver, heart, spleen or deep tissue. In some embodiments, the angiosarcoma forms in the skin of the head and neck.

In some embodiments, the rare cancer treated by a composition comprising a therapeutically effective amount of mebendazole comprises a rhabdomyosarcoma. In some embodiments, the rhabdomyosarcoma comprises an embryonal rhabdomyosarcoma. Embryonal rhabdomyosarcomas typically affect children in their first five years of life. The cells of an embryonal rhabdomyosarcoma comprise cells that resemble the developing muscle cells of a six to eight week embryo. In some embodiments, embryonal rhabdomyosarcomas comprise rhabdomyosarcomas of the head and neck area, bladder vagina, or in or around the prostate and testicles. In some embodiments, embryonal rhabdomyosarcomas comprise botryoid and spindle rhabdomyosarcomas. In some embodiments, the rhabdomyosarcoma comprises an alveolar rhabdomyosarcoma. Alveolar rhabdomyosarcomas typically affect all age groups equally. Alveolar rhabdomyosarcomas typically occur in the large muscles of the trunk, arms and legs. The cells of an alveolar rhabdomyosarcoma comprise cells that resemble those of normal muscle cells seen in a ten week old fetus. In some embodiments, the rhabdomyosarcoma comprises an anaplastic rhabdomyosarcoma.

In some embodiments, the rare cancer treated by a composition comprising a therapeutically effective amount of mebendazole comprises a neuroendocrine cancer. Neuroendocrine cancers arise from cells of the endocrine (hormonal) and nervous systems. In some embodiments, the neuroendocrine cancer comprises a carcinoid tumor. Carcinoid tumors are a type of slow growing tumor that comprise neuroendocrine cells and can arise at various places throughout the body. In some embodiments the carcinoid tumor comprises a small intestine tumor, an appendix tumor, a tumor of the rectum, a tumor of the bronchial system, a brain tumor, colon tumor, a stomach tumor, a pancreatic tumor, a liver tumor, a gallbladder tumor, a bile duct tumor, an ovarian tumor, a testicular tumor, a bladder tumor, a tumor of the prostate gland, a breast tumor, a kidney tumor, a thymic tumor, an eye tumor or an ear tumor. In some embodiments, the neuroendocrine cancer comprises a thymic cancer.

In some embodiments, the rare cancer treated by a composition comprising a therapeutically effective amount of mebendazole comprises a mesothelioma. Mesotheliomas comprise cancers that develop from the mesothelial, a thin layer of tissue lining lungs, abdomen or heart. In some embodiments, mesotheliomas affect the pleura that surrounds the lungs (pleural mesothelioma). In some embodiments, mesotheliomas affect the tissue of the abdomen (peritoneal mesothelioma). Risk factors for mesothelioma comprise asbestos exposure.

In some embodiments, the rare cancer treated by a composition comprising a therapeutically effective amount of mebendazole comprises a chordoma. Chordomas comprise cancerous tumors that occur along the spine. Chordomas are thought to arise from the cellular remnants of the notochord, the embryonic tissue that eventually forms the intervertebral disks. In some embodiments, the chordoma grows slowly, gradually extending into the surrounding bone and soft tissue. In some embodiments, the chordoma is relatively benign. In some embodiments, the chordoma is malignant.

In some embodiments, the rare cancer treated by a composition comprising a therapeutically effective amount of mebendazole comprises a pheochromocytoma, sometimes also referred to as a paraganglioma when the cancer arises in a chromaffin cell outside of the adrenal gland. A pheochromocytoma/paraganglioma is a rare tumor that develops in a chromaffin cell either in the adrenal gland or in the parasympathetic-associated tissues.

An exemplary, but non-limiting list of rare cancers that can be treated by the methods of the disclosure can be found at rarediseases.info.nih.gov/diseases/diseases-by-category/1/rare-cancers, the contents of which are herein incorporated by reference in their entirety. A list of rare cancers treatable by the methods of the disclosure is set forth in Table 1, below.

TABLE 1
Rare Cancers

5q- syndrome

A

| | |
|---|---|
| Acinic cell carcinoma | Acral lentiginous melanoma |
| Acromegaly | Acrospiroma |
| ACTH-secreting pituitary adenoma | Acute erythroid leukemia |
| Acute lymphoblastic leukemia | Acute lymphoblastic leukemia congenital sporadic |
| | aniridia |
| Acute megakaryoblastic leukemia | Acute monoblastic leukemia |
| Acute myeloblastic leukemia with maturation | Acute myeloblastic leukemia without maturation |
| Acute myeloid leukemia with abnormal bone marrow | Acute myeloid leukemia with inv3(p21; q26.2) or |
| eosinophils inv(16)(p13q22) or t(16; 16)(p13; q22) | t(3; 3)(p21; q26.2) |
| Acute myelomonocytic leukemia | Acute non lymphoblastic leukemia |
| Acute panmyelosis with myelofibrosis | Acute promyelocytic leukemia |
| Adenocarcinoid tumor | Adenocarcinoma of the appendix |
| Adenoid cystic carcinoma | Adenosarcoma of the uterus |
| Adrenal cancer | Adrenal medulla cancer |
| Adrenocortical carcinoma | Aggressive NK cell leukemia |
| Aicardi syndrome | Alveolar soft part sarcoma |
| Ameloblastic carcinoma | AML with myelodysplasia-related features |
| Anal cancer | Anaplastic astrocytoma |
| Anaplastic ependymoma | Anaplastic ganglioglioma |
| Anaplastic large cell lymphoma | Anaplastic oligoastrocytoma |
| Anaplastic oligodendroglioma | Anaplastic plasmacytoma |
| Anaplastic small cell lymphoma | Angiofollicular lymph hyperplasia |
| Angioimmunoblastic T-cell lymphoma | Angioma hereditary neurocutaneous |
| Angioma serpiginosum, autosomal dominant | Angioma serpiginosum, X-linked |
| Angiosarcoma of the breast | Angiosarcoma of the liver |
| Angiosarcoma of the scalp | Astroblastoma |
| Ataxia telangiectasia | Atrial myxoma, familial |
| Autoimmune lymphoproliferative syndrome | |

B

| | |
|---|---|
| B cell prolymphocytic leukemia | B-cell lymphoma |
| Bannayan-Riley-Ruvalcaba syndrome | Basal cell carcinoma, infundibulocystic |
| Basal cell carcinoma, multiple | Bazex-Dupre-Christol syndrome |
| Becker nevus syndrome | Bednar tumor |
| Benign metastasizing leiomyoma | Benign multicystic peritoneal mesothelioma |
| Bile duct cancer | Biliary tract cancer |
| Birt-Hogg-Dube syndrome | Bladder cancer, childhood |
| Blastic plasmacytoid dendritic cell | Bloom syndrome |
| Blue rubber bleb nevus syndrome | Bowen's disease |
| Brain stem cancer | Brain tumor, adult |
| Brain tumor, childhood | BRCA1 hereditary breast and ovarian cancer syndrome |
| BRCA2 hereditary breast and ovarian cancer syndrome | Breast cancer, childhood |
| Breast cancer, male | Brenner tumor of ovary |
| Brenner tumor of the vagina | Bronchial adenomas/carcinoids childhood |
| Burkitt lymphoma | Buschke Lowenstein tumor |

C

| | |
|---|---|
| Capillary hemangioblastoma | Carcinoid syndrome |
| Carcinoid tumor | Carcinoma of the vocal tract |
| Carcinoid tumor childhood | Carcinoma of unknown primary site, childhood |
| Carney complex | Carney triad |
| Carotid body tumor | Cartilaginous cancer |
| CDK4 linked melanoma | Central nervous system germinoma |
| Central neurocytoma | Cerebellar astrocytoma, childhood |
| Cerebellar liponeurocytoma | Cerebral astrocytoma, childhood |
| Cerebral sarcoma | Cerebral ventricle cancer |
| Cerebro-oculo-facio-skeletal syndrome | Cervical intraepithelial neoplasia |
| CHILD syndrome | Childhood acute lymphoblastic leukemia |
| Childhood brain stem glioma | Chondrosarcoma |
| Chordoid glioma of the third ventricle | Chordoma |
| Choriocarcinoma | Choroid plexus carcinoma |
| Choroid plexus papilloma | Chromophil renal cell carcinoma |
| Chronic lymphocytic leukemia | Chronic neutrophilic leukemia |
| Clear cell renal cell carcinoma | CLOVES syndrome |
| Cockayne syndrome type I, type II and type III | Collecting duct carcinoma |
| Common variable immunodeficiency | Costello syndrome |
| Cowden syndrome | Craniopharyngioma |
| Cronkhite-Canada disease | Cutaneous mastocytoma |
| Cutaneous T-cell lymphoma | |

D

| | |
|---|---|
| Deafness-lymphedema-leukemia syndrome | Dendritic cell tumor |
| Denys-Drash syndrome | Dermatofibrosarcoma protuberans |
| Desmoid tumor | Desmoplastic infantile ganglioglioma |
| Desmoplastic infantile astrocytoma | Desmoplastic small round cell tumor |
| Diamond-Blackfan anemia | Diaphyseal medullary stenosis with malignant fibrous |
| | histiocytoma |
| Diffuse astrocytoma | Diffuse cavernous hemangioma of the rectum |
| Diffuse gastric cancer | Diffuse Large B-Cell Lymphoma |
| Digestive System Melanoma | Disseminated peritoneal leiomyomatosis |
| Dysembryoplastic neuroepithelial tumor | Dyskeratosis congenita |
| Dyskeratosis congenita autosomal dominant | Dyskeratosis congenita autosomal recessive |
| Dyskeratosis congenita X-linked | Diffuse intrinsic pontine glioma |

E

| | |
|---|---|
| Eccrine mucinous carcinoma | Eccrine porocarcinoma |
| Embryonal carcinoma | Embryonal sarcoma |
| Embryonal tumor with multilayered rosettes | Enchondroma |
| Endemic Kaposi sarcoma | Endometrial stromal sarcoma |
| Enteropathy-associated T-cell lymphoma | Ependymoma |
| Epithelial-myoepithelial carcinoma | Esophageal cancer |
| Esophageal cancer, childhood | Essential thrombocythemia |
| Ewing sarcoma (Ewing's sarcoma) | Ewing's family of tumors |
| Extragonadal germ cell tumor | Extragonadal germ cell tumor |

F

| | |
|---|---|
| Fallopian tube cancer | Fallopian tube cancer |
| Familial adenomatous polyposis | Familial colorectal cancer |
| Familial cylindromatosis | Familial hyperaldosteronism type 2 |
| Familial pancreatic cancer | Familial platelet disorder with associated myeloid |
| | malignancy |
| Familial prostate cancer | Familial stomach cancer |
| Familial Wilms tumor 2 | Fanconi anemia |
| Fibrolamellar carcinoma | Fibrosarcoma |
| Follicular lymphoma | Frasier syndrome |
| Functioning pancreatic endocrine tumor | |

G

| | |
|---|---|
| Gallbladder cancer | Gangliocytoma |
| Ganglioglioma | Gardner syndrome |
| Gastric lymphoma | Gastro-enteropancreatic neuroendocrine tumor |
| Gastrointestinal Stromal Tumors | Giant cell tumor of bone |
| Giant congenital nevus | Glassy cell carcinoma of the cervix |
| Glioblastoma | Glioma |
| Gliosarcoma | Glomus jugulare tumors |
| Glomus tympanicum tumor | Glomus vagale tumor |
| Glucagonoma | Glucagonoma syndrome |
| Goblet cell carcinoid | Granular cell tumor |
| Granulomatous slack skin disease | Granulosa cell tumor of the ovary |
| Gray zone lymphoma | Gynandroblastoma |

H

| | |
|---|---|
| Hairy cell leukemia | Heart tumor |
| Hemangioblastoma | Hemangioendothelioma |
| Hemangioma thrombocytopenia syndrome | Hemangiopericytoma |
| Hemi 3 syndrome | Hepatoblastoma |
| Hereditary diffuse gastric cancer | Hereditary leiomyomatosis and renal cell cancer |
| Hereditary paraganglioma-pheochromocytoma | Hereditary multiple osteochondromas |
| Hidradenocarcinoma | Hereditary renal cell carcinoma |
| Hodgkin lymphoma, childhood | Hodgkin lymphoma |
| Hyaline fibromatosis syndrome | Hurthle cell thyroid cancer |
| Hypopharyngeal cancer | Hyperparathyroidism-jaw tumor syndrome |

I

| | |
|---|---|
| Indolent B cell lymphoma | Infantile myofibromatosis |
| Inflammatory breast cancer | Inflammatory linear verrucous epidermal nevus |
| Inflammatory myofibroblastic tumor | Insulinoma |
| Intrahepatic cholangiocarcinoma | Intraneural perineurioma |
| Intraocular melanoma | |

J

| | |
|---|---|
| Juvenile myelomonocytic leukemia | Juvenile polyposis syndrome |

K

| | |
|---|---|
| Kaposi sarcoma | Kaposiform Hemangioendothelioma |
| Keratosis palmoplantaris adenocarcinoma of the colon | Kidney cancer, childhood |
| Klatskin tumor | Krukenberg carcinoma |

L

| | |
|---|---|
| Langerhans cell sarcoma | Large granular lymphocyte leukemia |
| Laryngeal cancer | Laryngeal cancer, childhood |
| Ledderhose disease | Leiomyosarcoma |
| Lentigo maligna melanoma | LEOPARD syndrome |
| Leukemia subleukemic | Leukemia, B-cell, chronic |
| Leukemia, T-cell, chronic | Lhermitte-Duclos disease |
| Li-Fraumeni syndrome | Linear nevus sebaceous syndrome |
| Lip and oral cavity cancer | Lipoblastoma |
| Liposarcoma | Lung adenocarcinoma |
| Lymph Node Neoplasm | Lymphoblastic lymphoma |
| Lymphoma AIDS related | Lymphoma, gastric non Hodgkins type |
| Lymphoma, large-cell | Lymphoma, large-cell, immunoblastic |
| Lymphomatoid papulosis | Lymphosarcoma |

M

| | |
|---|---|
| Macrocephaly-capillary malformation | Maffucci syndrome |
| Mahvash disease | Malignant cylindroma |
| Malignant eccrine spiradenoma | Malignant germ cell tumor |
| Malignant melanoma, childhood | Malignant mesenchymoma |
| Malignant mesothelioma | Malignant mixed Mullerian tumor |
| Malignant peripheral nerve sheath tumor | Malignant Teratocarcinosarcoma |
| Mantle cell lymphoma | McCune-Albright syndrome |
| Mediastinal endodermal sinus tumors | Medulloblastoma |
| Medulloblastoma, childhood | Melanocytic lesions of CNS |
| Melanoma astrocytoma syndrome | Melanoma, familial |
| Meningioma | Merkel cell carcinoma |
| Metaplastic carcinoma of the breast | Metastatic insulinoma |
| Metastatic squamous neck cancer with occult primary | Microcystic adnexal carcinoma |
| Microcystic lymphatic malformation | Mosaic variegated aneuploidy syndrome |
| Mucoepidermoid carcinoma | Muir-Torre syndrome |
| Multicentric Castleman Disease | Multiple endocrine neoplasia type 1, 2A and 2B |
| Multiple familial trichoepithelioma 1 and 2 | Multiple fibrofolliculoma familial |
| Multiple myeloma | Mycosis fungoides |
| Myelocytic leukemia-like syndrome, familial, chronic | Myelofibrosis |
| Myeloid leukemia | Myeloid sarcoma |
| Myoepithelial carcinoma | Myxoid liposarcoma |

N

| | |
|---|---|
| N syndrome | Nasal cavity cancer, childhood |
| Nasopharyngeal cancer, childhood | Nasopharyngeal carcinoma |
| Neural crest tumor | Neuroblastoma |
| Neurocutaneous melanosis | Neuroendocrine carcinoma of the cervix |
| Neuroepithelioma | Neurofibromatosis type 2 |
| Neurofibromatosis-Noonan syndrome | Neurofibrosarcoma |
| Nevoid basal cell carcinoma syndrome | Nevus comedonicus syndrome |
| Nevus of Ito | Nijmegen breakage syndrome |
| Nodular melanoma | Non functioning pancreatic endocrine tumor |
| Non-Hodgkin lymphoma, childhood | Non-Hodgkin lymphoma, during pregnancy |
| Non-involuting congenital hemangioma | Non-small cell lung cancer, childhood |
| Nonseminomatous germ cell tumor | Noonan syndrome (1-6) |

O

| | |
|---|---|
| Ocular melanoma | Olfactory neuroblastoma |
| Oligoastrocytoma | Oligodendroglioma |
| Ollier disease | Onychocytic matricoma |
| Optic pathway glioma | Oral cancer |
| Oral squamous cell carcinoma | Orbital lymphangioma |
| Orbital lymphoma | Oropharyngeal cancer, adult |
| Oropharyngeal cancer, childhood | Oslam syndrome |
| Osteofibrous dysplasia | Osteosarcoma |
| Ovarian cancer | Ovarian carcinosarcoma |
| Ovarian epithelial cancer | Ovarian germ cell tumor |
| Ovarian low malignant potential tumor | |

P

| | |
|---|---|
| Paget disease of the breast | Paget disease, extramammary |
| Painful orbital and systemic neurofibromas-marfanoid | Pancreatic adenoma |
| habitus syndrome | |
| Pancreatic cancer, childhood | Pancreatic islet cell tumors |
| Pancreatoblastoma | Papillary cystadenocarcinoma |
| Papillary renal cell carcinoma | Papillary thyroid carcinoma |
| Paraganglioma and gastric stromal sarcoma | Paranasal sinus cancer, adult |
| Paranasal sinus cancer, childhood | Paraneoplastic cerebellar degeneration |
| Parathyroid cancer, childhood | Parathyroid carcinoma |
| Pediatric T-cell leukemia | Penile cancer, adult |
| Penile cancer, childhood | Peripheral T-cell lymphoma |
| Perlman syndrome | Peutz-Jeghers syndrome |
| PHACE syndrome | Pheochromocytoma |
| Pheochromocytoma, childhood | Philadelphia-negative chronic myeloid leukemia |
| Phyllodes tumor of the breast | Phyllodes tumor of the prostate |
| Pilocytic astrocytoma | Pilomatrixoma |
| Pineal parenchymal tumors of intermediate | Pineoblastoma |
| differentiation | |
| Pineoblastoma, childhood | Pituitary cancer |
| Plasma cell leukemia | Pleomorphic xanthoastrocytoma |
| Pleuropulmonary blastoma | Plexosarcoma |
| POEMS syndrome | Polycythemia vera |
| Polyembryoma | Polymorphous low-grade adenocarcinoma |
| Primary central nervous system lymphoma | Primary effusion lymphoma |
| Primary liver cancer | Primary malignant melanoma of the cervix |
| Primary malignant melanoma of the conjunctiva | Primary melanoma of the central nervous system |
| Proliferating trichilemmal cyst | Proteus syndrome |
| Proteus-like syndrome | Pseudomyxoma peritonei |

R

| | |
|---|---|
| Radiation induced angiosarcoma of the breast | Radiation induced cancer |
| Radiation induced meningioma | Rare adenocarcinoma of the breast |
| Rectal cancer, childhood | Renal carcinoma, familial |
| Renal cell carcinoma 4 | Retinoblastoma |
| Retroperitoneal liposarcoma | Rhabdoid tumor |
| Rhabdomyosarcoma alveolar | Rhabdomyosarcoma embryonal |
| Richter syndrome | Ring dermoid of cornea |
| Rombo syndrome | |

S

| | |
|---|---|
| Sacrococcygeal Teratoma | Saethre-Chotzen syndrome |
| Salivary gland cancer, childhood | Salivary gland cancer, adult |
| Sarcoma botryoides | Schinzel Giedion syndrome |
| Schwannomatosis | Secretory breast carcinoma |
| Sertoli-leydig cell tumors | Severe congenital neutropenia autosomal recessive 3 |
| Sezary syndrome | Shwachman-Diamond syndrome |
| Sideroblastic anemia pyridoxine-refractory autosomal | Simpson-Golabi-Behmel syndrome |
| recessive | |
| Sinonasal undifferentiated carcinoma | Sinus cancer |
| Skin cancer, non melanoma, childhood | Small cell carcinoma of the bladder |
| Small cell lung cancer, childhood and adult | Small intestine cancer, childhood |
| Small intestine cancer | Soft tissue sarcoma |
| Soft tissue sarcoma childhood | Somatostatinoma |
| Sotos syndrome | Splenic neoplasm |
| Stomach cancer | Stomach cancer, childhood |
| Subcutaneous panniculitis-like T-cell lymphoma | Subependymal giant cell astrocytoma |
| Subependymoma | Superficial spreading melanoma |
| Supraglottic laryngeal cancer | Supratentorial primitive neuroectodermal tumor |
| Supratentorial primitive neuroectodermal tumors, | Supraumbilical midabdominal raphe and facial |
| childhood | cavernous hemangiomas |
| Synovial cancer | Synovial sarcoma |

T

| | |
|---|---|
| T-cell lymphoma 1A | T-cell/histiocyte rich large B cell lymphoma |
| Teratoma with malignant transformation | Testicular cancer |
| Testicular cancer, childhood | Testicular seminoma |
| Testicular yolk sac tumor | Thoracolaryngopelvic dysplasia |
| Thymic epithelial tumor | Thymoma, childhood |
| Thyroid cancer, anaplastic | Thyroid cancer, childhood |
| Thyroid cancer, follicular | Thyroid cancer, medullary |
| Tongue cancer | Transient myeloproliferative syndrome |
| Transitional cell cancer of the renal pelvis and ureter | Transitional cell carcinoma |
| Trichofolliculoma | Trophoblastic tumor placental site |
| Tuberous sclerosis | Tufted angioma |
| Turcot syndrome | Tylosis with esophageal cancer |
| Tyrosinemia type 1 | |

U

| | |
|---|---|
| Undifferentiated pleomorphic sarcoma | Unicentric Castleman disease |
| Urachal adenocarcinoma | Urachal cancer |
| Urethral cancer | Uterine Carcinosarcoma |
| Uterine sarcoma | |

V

| | |
|---|---|
| Vaginal cancer | Verrucous nevus acanthokeratolytic |
| VIPoma | Visual pathway and hypothalamic glioma, childhood |
| Von Hippel-Lindau disease | Vulvar cancer |

W

| | |
|---|---|
| WAGR syndrome | Waldenstrom macroglobulinemia |
| Werner's syndrome | White sponge nevus of cannon |
| Wilms tumor and radial bilateral aplasia | Wilms' tumor |
| Wiskott Aldrich syndrome | WT limb blood syndrome |

X

| | |
|---|---|
| X-linked lymphoproliferative syndrome | X-linked lymphoproliferative syndrome 1 |
| Xeroderma pigmentosum | |

Z

| | |
|---|---|
| Zollinger-Ellison syndrome | Zuska's disease |

### Treatment of Cancer

In some embodiments of the methods of treating cancer of the disclosure, the administration of the composition comprising a therapeutically effective amount of mebendazole comprises a cancer monotherapy.

In some embodiments of the methods of treating cancer of the disclosure, the administration of the composition comprising a therapeutically effective amount of mebendazole comprises is part of a cancer combinational therapy. The composition comprising mebendazole can be combined with additional chemotherapeutic agents, cancer therapeutic agents, cancer combination therapies, targeted small molecules, biologics such as antibodies or cancer treatments.

In some embodiments of the methods of treating cancer of the disclosure, the treatment of cancer further comprises the administration of both the composition comprising a therapeutically effective amount of mebendazole and one or more additional cancer treatments or therapeutic agents. Treatments of the rare cancers of the disclosure typically comprise surgical resection of the cancer. In some embodiments, especially those embodiments wherein the cancer is an early stage cancer that has not yet spread to surrounding tissues or metastasized throughout the body, treatment of the cancer may consist of complete surgical resection of the cancer. Alternatively, or in addition, treatment of the cancer may further comprise chemotherapy, radiation therapy or a combination thereof. In some embodiments, treatment of a cancer of the disclosure may comprise neoadjuvant chemotherapy, i.e. chemotherapy that occurs before surgical intervention to remove the cancer. In some embodiments, in particular those embodiments wherein the cancer is a neuroendocrine cancer, treatment may comprise hormone therapy.

In some embodiments of the methods of treating cancer of the disclosure, the cancer treatment further comprises administering a second therapeutic agent or combination of therapeutic agents. In some embodiments, the second therapeutic agent or combination of therapeutic agents comprises a chemotherapeutic agent, a combination of chemotherapeutic agents, or a chemotherapeutic agent combined with an additional cancer therapy. In some embodiments, the one or more additional therapeutic agents comprises a Maytansinoid or an analog or derivative thereof. In some embodiments, the Maytansinoid comprises Maytansine, Maytansinol, or derivatives or analogs thereof. In some embodiments, the one or more additional therapeutic agents comprises a Calicheamicin, or a derivative or an analog thereof. In some embodiments, the one or more additional therapeutic agents comprises an Auristatin or a derivative or analog thereof. Exemplary but non-limiting auristatins comprise Monomethyl auristatin E, Monomethyl auristatin F, Dolastatin 10, Dolastatin 15, or analogs or derivatives thereof. In some embodiments, the one or more additional therapeutic agents comprises a Halichondrin and analogs or a derivative thereof. In some embodiments, the one or more additional therapeutic agents comprises a Hemiasterlin or an analog or a derivative thereof. In some embodiments, the one or more additional therapeutic agents comprises a Crytophycin or an analog or a derivative thereof. In some embodiments, the one or more additional therapeutic agents comprises a Spongistatin or an analog or a derivative thereof. In some embodiments, the one or more additional therapeutic agents comprises an Alkoyamine or an analog or a derivative thereof. In some embodiments, the one or more additional therapeutic agents comprises a Sesterterpenoid or an analog or a derivative thereof.

In some embodiments, second therapeutic agent or combination of therapeutic agents other than mebendazole targets cellular pathways that are upregulated or critical in rapidly dividing cancer cells. Exemplary agents and pathways comprise cell cycle checkpoint inhibitors, antimitotic agents, pro-apoptotic agents, DNA damaging agents or inhibitors of the DNA damage response pathway. Exemplary but non-limiting chemotherapeutic agents which may, in some embodiments, be administered in combination with the composition comprising mebendazole of the disclosure are shown in Table 2.

TABLE 2
Chemotherapeutic Agents

| generic name | brand name ® | other brand names ®/formulations |
|---|---|---|
| 5-fluoruracil | Adrucil | |
| Abemaciclib | Verzenio | |
| Abiraterone acetate | Zytiga | |
| Afatinib | Gilotrif | |
| Aldesleukin | Proleukin | |
| Alitretinoin | Panretin | |
| Altretamine | Hexalen | |
| Amifostine | Ethyol | |
| Anastrozole | Arimidex | |
| arsenic trioxide | Trisenox | |
| Asparaginase | Elspar | |
| Asparaginase | Erwinaze | |
| Erwinia chrysanthemi | | |
| Axinitib | Inlyta | |
| Azacitidine | Vidaza | |
| BCG | TheraCys BCG, | TICE BCG, Bacillus Calmette-Guerin |
| | | vaccine |
| Bendamustine hydrochloride | Treanda | |
| Bexarotene | Targretin | |
| Bicalutamide | Casodex | |
| Bilnostat | Beleodaq | |
| Bleomycin | Blenoxane | |
| Bortezomib | Velcade | |
| Bosutinib | Bosulif | |
| Buslfan | Buslfex | |
| Busulfan | Myleran | |
| Cabazitaxel | Jevtana | |
| Cabozantinib | Cometriq | |
| capecitabine | Xeloda | |
| carboplatin | Paraplatin | |
| Carfilzomib | Kyprolis | |
| Carmustine | BiCNU | |
| Carmustine | Gliadel Wafer | |
| Ceretinib | Zykadia | |
| Chlorambucil | Leukeran | |
| Cisplatin | Platinol | PlatinolAQ |
| Cladribine | Leustatin | |
| Clofarabine | Clolar | |
| Cobemetinib | Cotellic | |
| Crizotinib | Xalkori | |
| Cyclophosphamide | Neosar | 4-hydroperoxycyclophosphamide (4- |
| | | HC); Pergamid |
| Cytarabine | CytosarU | DepoCyt, Cytarabine lipid complex |
| Cytoxan | Cytoxan | Cyclophosphamide |
| Dabrafenib | Taflinar | |
| Dactinomycin | Cosmegen | |
| dasatinib | Sprycel | |
| Daunorubicin | Cerubidine | |
| Daunorubicin | DaunoXome | Daunorubicin lipid complex |
| Dacarbazine | DTIC | |
| Decitabine | Dacogen | |
| Degarelix | Firmagon | |
| Denileukin diftitox | Ontak | |
| Dexamethasone | Decadron | Dexamethosone Intensol, Dexpak |
| | | Taperpak |
| Docetaxel | Docefrez | |
| Doxorubicin | Adriamycin | Doxorubicin lipid complex, Doxil, Rubex |
| Ellence epirubicin | Ellence | |
| Eloxatin oxaliplatin | Eloxatin | |
| Enzalutamide | Xtandi | |
| Eribulin | Galaven | |
| Erlotinib | Tarceva | |
| Estramustine | Emcyt | |
| Etoposide | Etopophos | Toposar, Vepesid |
| Everolimus | Afinitor | Zortress, Afinitor Disperz |
| Exemestane | Aromasin | |
| Filgrastim | Neulasta | Pegfilgrastim, Neupogen |
| Fludarabine | Fludara | |
| Flutamide | Eulexin | |
| FUDR floxuridine | FUDR | |
| Fulvestrant | Faslodex | |
| Gefitinib | Iressa | |
| Gemcitabine | Gemzar | |
| Goserelin | Zoladex | |
| HDAC | High Dose | |
| | Cytarabine | |
| Histrelin | Supprelin LA | Histrelin implant, Vantas |
| Hydroxyurea | Droxia | Hydrea |
| Iapatinib | Tykerb | |
| Ibrutinib | Imbruvica | |
| Idarubicin | Idamycin PFS | |
| idelalisib | Zydelig | |
| Ifosfamide | Ifex | |
| Imatinib Mesylate | Gleevec | |
| interferon alpha-2a | Intron A alfab | RoferonA alfaa |
| Irinotecan | Camptosar | |
| Ixabepilone | Ixempra | |
| Lapatinib | Tykerb | Tyverb, Lapatinib ditosylate |
| lenalidomide | Revlimid | |
| Lenvatinib mesylate | Lenvima | |
| Lanreotide acetate | Somatuline | |
| | Depot | |
| Letrozole | Femara | |
| leucovorin | Wellcovorin IV | |
| Leuprolide | Eligard | Lupron, Lupron Depot, Lupron |
| | | DepotPED |
| Lomustine | CeeNU | |
| Mechlorethamine | Mustargen | |
| Megestrol | Megace | |
| Melphalan | Alteran | |
| Mercaptopurine | Purinethol | |
| Mesna | Mesnex | |
| Methotrexate | Abitrexate | Folex, Mexate, Rheumatrex, Trexall |
| Mitomycin | Mutamycin | |
| Mitotane | Lysodren | |
| mitoxantrone | Novantrone | |
| Nelarabine | Arranon | |
| Nilandron nilutamide | Nilandron | |
| Octreotide | Sandostatin | Sandostatin LAR depot |
| Olaparib | Lynparza | |
| Omacetraxine | synribo | |
| Paclitaxel | Abraxane | Onxol, Taxol |
| Palbociclib | Ibrance | |
| Pamidronate | Aredia | |
| Panitumumab | Vectabix | |
| Panobinostat | Farydak | |
| Pazapanib | Votrient | |
| Pegaspargase | Oncaspar | |
| Peginterferon alpha-2b | Sylatron | |
| Pemetrexed | Alimta | |
| Pentostatin | Nipent | |
| Pomalidomide | Pomalyst | |
| Ponatinib | Iclusig | |
| Pralatrexate | Flolotyn | |
| Prednisone | Predisone | Sterapred, Sterapred DS |
| | intensol | |
| Procarbazine | Matulane | |
| Raltitrexed | Tomudex | |
| Radium 223 dichloride | Xofigo | |
| Regorafenib | Stivarga | |
| Romidepsin | Istodax | |
| Ruxolitinib | Jakafi | |
| Sargramostim | Leukine | |
| Siltuximab | Sylvant | |
| Sonidegib | Odomzo | |
| Soragenib | Nexava | |
| Streptozocin | Zanosar | |
| Strontium 89 chloride | Metastron | |
| Sunitinib | Sutent | |
| Tamoxifen | Nolvadex | Soltamox |
| Temozolomide | Temodar | |
| Temsirolimus | Torisel | |
| Teniposide | Vumon | |
| Thalidomide | Thalomid | |
| Thioguanine | Tabloid | |
| Thiotepa | Tepadina | Thioplex |
| Topotecan | Hycamtin | |
| Toremifene | Fareston | |
| Trabectedin | Yondelis | |
| Trametinib | Mekinist | |
| Tretinoin | Sanoid | |
| Trifluridine and Tipiracil | Lonsurf | |
| Triptorelin | Trelstar | |
| Talrubicin intravesical | Valstar | |
| Vandetabib | Caprelsa | |
| vemurafenib | Zelboraf | |
| vinblastine | Velban | |
| Vincristine | Marqibo Kit | Vincristine lipid complex, Oncovin, |
| | | Vincasar PFS, Vincrex |
| vinorelibine | Navelbine | |
| Vismodegib | Erivedge | |
| Vorinostat | Zolinza | |
| Ziv-aflibercept | Zaltrap | |
| Zoledronic acid | Zometra | Reclast |
| Alectinib | Alecensa | |
| Ixazomib | Ninlaro | |
| Nilotinib | Tasigna | |
| Osimertinib | Tagrisso | |
| Venetoclax | Venclexta | |
| Ribociclib | Kisqali | |
| Enasidenib | Idhifa | |
| Rucaparib | Rubraca | |
| Niraparib | Zejula | |
| Copanlisib | Aliqopa | |
| Neratinib | Nerlynx | |
| Brigatinib | Alunbrig | |
| Midostaurin | Rydapt | |
| Vindesine | Eldisine | Vindesine sulfate |
| Lometrexol | | |
| Satraplatin | | |
| Larotaxel | | |
| Rapamycin | Sirolimus | Rapamune |
| Temsirolimus | Torisel | |
| Ridaforolimus | Deforolimus | MK-8669 |

In some embodiments of the methods of treating cancer of the disclosure comprising administering a therapeutically effective amount of a composition comprising mebendazole, the treatment further comprises the administration of a small molecule targeted therapy. In some embodiments, the small molecule targeted therapy may comprise Alectinib (Alecensa), Ixazomib (Ninlaro), Nilotinib (Tasigna), Osimertinib (Tagrisso), Venetoclax (Venclexta), Ribociclib (Kisqali), Enasidenib (Idhifa), Rucaparib (Rubraca), Niraparib (Zejula), Copanlisib (Aliqopa), Neratinib (Nerlynx), Brigatinib (Alunbrig), Midostaurin (Rydapt) or a combination thereof.

In some embodiments of the methods of treating cancer of the disclosure comprising administering a therapeutically effective amount of a composition comprising mebendazole, the treatment further comprises the administration of a combination chemotherapy. Exemplary but non-limiting examples of combination chemotherapies are disclosed in Table 3 below. The ordinarily skilled artisan will understand that there are variations known in the art of the combination chemotherapies disclosed in table 3. For example, combinations which substitute one steroid for another similar steroid may still be called by the same abbreviation.

TABLE 3
Combination Chemotherapies

| Name | Combination |
|---|---|
| 7 + 3 | 7 days of standard dose Cytarabine; 3 days of |
| | Daunorubicin, Doxorubicin, Idarubicin or |
| | Mitoxantrone |
| ABVD | Doxorubicin, Bleomycin, Vinblastine, Dacarbazine |
| AC | Doxorubicin, Cyclophosphamide |
| AD | Doxorubicin, Dacarbazine |
| ADE | Cytarabine, Daunorubicin, Etoposide |
| ADOC | Cisplatin, Doxorubicin, Vincristine, Cyclophosphamide |
| BEACOPP | Bleomycin, Etoposide, Doxorubicin, Cyclophosphamide, |
| | Vincristine, Procarbazine, Prednisone |
| BEP | Bleomycin, Etoposide, Cisplatin |
| CAF | Cyclophosphamide, Doxorubicin, 5-Fluorouracil (5-FU) |
| CAPIRI | Capecitabine, Irinotecan |
| CAPOX | Capecitabine, Oxaliplatin |
| CB | Cetuximab, Bevacizumab |
| CBI | Cetuximab, Bevacizumab, Irinotecan |
| CEF | Cyclophosphamide, Epirubicin, 5-FU |
| CEPP | Cyclophosphamide, Etoposide, Procarbazine, Prednisone |
| CFAR | Cyclophosphamide, Fludarabine, Alemtuzumab, Rituximab |
| CHOP | Cyclophosphamide, Doxorubicin, Vincristine, Prednisolone |
| CHOP + R | Cyclophosphamide, Doxorubicin, Vincristine, Prednisolone, |
| (R-CHOP) | Rituximab |
| CIM | Cisplatin, Ifosfamide, Mesna |
| CLAG | Cladribine, Cytarabine, |
| CLAG-M | Cladribine, Cytarabine, Mitoxantrone |
| CMC | Cladribine, Mitoxantrone, Cyclophosphamide |
| CMF | Cyclophosphamide, Methotrexate, 5-FU |
| COI | Capecitabine, Oxaliplatin, Irinotecan |
| CVD | Cisplatin, Vinblastine, Dacarbazine |
| CVP | Cyclophosphamide, Vincristine, Prednisone |
| DHAP | Cisplatin, Cytarabine, Dexamethasone |
| DVD | Doxorubicin, Vincristine, Dexamethasone |
| ECF | Epirubicin, Cisplatin, 5-FU |
| ECX | Epirubicin, Oxaliplatin, Capecitabine |
| EOF | Epirubicin, Oxaliplatin, 5-FU |
| EOX | Epirubicin, Oxaliplatin, Capecitabine |
| EP | Etoposide, Cisplatin |
| EPOCH | Etoposide, Prednisone, Vincristine, Cyclophosphamide, |
| | Doxorubicin |
| EPOCH + R | Etoposide, Prednisone, Vincristine, Cyclophosphamide, |
| | Doxorubicin, Rituximab |
| ESHAP | Etoposide, Methylprednisolone, Cisplatin, Cytarabine |
| FAMTX | Methotrexate, 5-FU, Doxorubicin |
| FC | Fludarabine, Cyclophosphamide |
| FCR | Fludarabine, Cyclophosphamide, Rituximab |
| FEC | 5-FU, Epirubicin, Cyclophosphamide |
| FLAG-IDA | Fludarabine, Cytarabine, Idarubicin |
| FLO | 5-FU, Leucovorin, Oxaliplatin |
| FLOX | 5-FU, Leucovorin, Oxaliplatin |
| FOLFIRI | Irinotecan, 5-FU, Leucovorin |
| FOLFOX | 5-FU, Leucovorin, Oxaliplatin |
| FOLFOXIRI | 5-FU, Leucovorin, Oxaliplatin, Irinotecan |
| GEMOX-B | Gemcitabine, Oxaliplatin, Bevacizumab |
| GVD | Gemcitabine, Vinorelbine, Doxorubicin |
| Hyper- | Cyclophosphamide, Doxorubicin, Vincristine, |
| CVAD | Dexamethasone, Mesna, Methotrexate, Leucovorin, |
| | Cytarabine |
| ICE | Ifosfamide, Carboplatin, Etoposide, Mesna |
| ICE-V | Ifosfamide, Carboplatin, Etoposide, Mesna, Vincristine |
| IFL | Irinotecan, 5-FU, Leucovorin |
| IROX | Irinotecan, Oxaliplatin |
| LV5FU2 | 5-FU, Leucovorin |
| LV5FU-P | Irinotecan, 5-FU, Leucovorin |
| MAID | Mesna, Doxorubicin, Ifosfamide, Dacarbazine |
| MFL | Methotrexate, 5-FU, Leucovorin |
| MINE | Mesna, Ifosfamide, Mitoxantrone, Etoposide |
| MOPP | Mechlorethamine, Vincristine, Prednisone, Procarbazine |
| MP | Melphalan, Prednisone |
| MPV | Methotrexate, Procarbazine, Vincristine |
| MVAC | Methotrexate, Vinblastine, Doxorubicin, Cisplatin |
| OFF | Oxaliplatin, 5-FU, Leucovorin |
| PAC | Cisplatin, Doxorubicin, Cyclophosphamide |
| PAD | Bortezomib, Dexamethasobne, Doxorubicin |
| PCR | Pentostatin, Cyclophosphamide, Rituximab |
| PCV | Procarbazine, Lomustine, Vincristine |
| R-MPV | Methotrexate, Procarbazine, Vincristine, Rituximab, |
| | Leucovorin |
| R-GemOx | Rituximab, Gemcitabine, Oxaliplatin |
| R-CVP | Cyclophosphamide, Vincristine, Prednisone, Rituximab |
| R-FCM | Rituximab, Cyclophosphamide, Fludabarine, Mitoxantrone |
| RICE | Ifosfamide, Carboplatin, Etoposide, Mesna, Rituximab |
| TAC | Docetaxel, Doxorubicin, Cyclophosphamide |
| TC | Docetaxel, Cyclophosphamide |
| TCH | Docetaxel, Carboplatin, Trastuzumab |
| TIP | Paclitaxel, Ifosfamide, Mesna, Cisplatin |
| TPC | Trastuzumab, Paclitaxel, Carboplatin |
| TPF | Docetaxel, Cisplatin, 5-FU |
| VAD | Vincristine, Doxorubicin, Dexamethosone |
| VIP | Etoposide, Vinblastine, Ifosfamide, Cisplatin, Mesna |
| VMP | Bortezomib, Melphalan, Prednisone |
| VMPT | Bortezomib, Melphalan, Prednisone, Thalidomide |
| XELIRI | Capecitabine, Irinotecan |
| XELOX | Capecitabine, Oxaliplatin |

In some embodiments of the methods of the disclosure, the methods further comprise administering a combination chemotherapy. In some embodiments, the combination chemotherapy comprises a 7+3 combination chemotherapy. Exemplary cancers treated by a 7+3 combination chemotherapy include but are not limited to acute myelogenous leukemia. In some embodiments, the combination chemotherapy comprises an ABVD combination chemotherapy. Exemplary cancers treated by an ABVD combination chemotherapy include but are not limited to Hodgkin lymphoma. In some embodiments, the combination chemotherapy comprises an AC or CMF combination chemotherapy. Exemplary cancers treated by an AC or CMF combination chemotherapy include but are not limited to breast cancers. In some embodiments, the combination chemotherapy comprises an AD combination chemotherapy. Exemplary cancers treated by an AD combination chemotherapy include but are not limited to sarcomas. In some embodiments, the combination chemotherapy comprises an ADE combination chemotherapy. Exemplary cancers treated by an ADE combination chemotherapy include but are not limited to acute myelogenous leukemias. In some embodiments, the combination chemotherapy comprises an ADOC combination chemotherapy. Exemplary cancers treated by an ADOC combination chemotherapy include but are not limited to thymoma. In some embodiments, the combination chemotherapy comprises a BEACOPP combination chemotherapy. Exemplary cancers treated by a BEACOPP combination chemotherapy include but are not limited to Hodgkin lymphomas. In some embodiments, the combination chemotherapy comprises a BEP combination chemotherapy. Exemplary cancers treated by a BEP combination chemotherapy include but are not limited to testicular cancers. In some embodiments, the combination chemotherapy comprises a CAF or CEF combination chemotherapy. Exemplary cancers treated by a CAF or CEF combination chemotherapy include but are not limited to breast cancers. In some embodiments, the combination chemotherapy comprises a CAPIRI, CAPOX, CB, CBI or COI combination chemotherapy. Exemplary cancers treated by a CAPIRI, CAPOX, CB, CBI or COI combination chemotherapy include but are not limited to colorectal cancers. In some embodiments, the combination chemotherapy comprises a CEPP, CHOP or R-CHOP combination chemotherapy. Exemplary cancers treated by a CEPP, CHOP or R-CHOP combination chemotherapy include but are not limited to non-Hodgkin lymphomas. In some embodiments, the combination chemotherapy comprises a CFAR or CMC combination chemotherapy. Exemplary cancers treated by a CFAR or CMC combination chemotherapy include but are not limited to chronic lymphocytic leukemias. In some embodiments, the combination chemotherapy comprises a CIM combination chemotherapy. Exemplary cancers treated by a CIM combination chemotherapy include but are not limited to uterine sarcomas. In some embodiments, the combination chemotherapy comprises a CLAG or a CLAG-M combination chemotherapy. Exemplary cancers treated by a CLAG or a CLAG-M combination chemotherapy include but are not limited to acute myelogenous leukemias. In some embodiments, the combination chemotherapy comprises a CVD combination chemotherapy. Exemplary cancers treated by a CVD combination chemotherapy include but are not limited to melanomas. In some embodiments, the combination chemotherapy comprises a CVP combination chemotherapy. Exemplary cancers treated by a CVP combination chemotherapy include but are not limited to chronic lymphocytic leukemias and non-Hodgkin lymphomas. In some embodiments, the combination chemotherapy comprises a DHAP combination chemotherapy. Exemplary cancers treated by a DHAP combination chemotherapy include but are not limited to lymphomas. In some embodiments, the combination chemotherapy comprises a DVD combination chemotherapy. Exemplary cancers treated by a DVD combination chemotherapy include but are not limited to multiple myelomas. In some embodiments, the combination chemotherapy comprises an ECF, ECX, EOF or EOX combination chemotherapy. Exemplary cancers treated by an ECF, ECX, EOF or EOX combination chemotherapy include but are not limited to esophageal cancers and gastric cancers. In some embodiments, the combination chemotherapy comprises an EP combination chemotherapy. Exemplary cancers treated by an EP combination chemotherapy include but are not limited to testicular cancers and thymomas. In some embodiments, the combination chemotherapy comprises an EPOCH, EPOCH+R or ESHAP combination chemotherapy. Exemplary cancers treated by an EPOCH, EPOCH+R or ESHAP combination chemotherapy include but are not limited to non-Hodgkin lymphomas. In some embodiments, the combination chemotherapy comprises a FAMTX combination chemotherapy. Exemplary cancers treated by a FAMTX combination chemotherapy include but are not limited to gastric cancers. In some embodiments, the combination chemotherapy comprises a FC or FCR combination chemotherapy. Exemplary cancers treated by a FC or FCR combination chemotherapy include but are not limited to chronic lymphocytic leukemias. In some embodiments, the combination chemotherapy comprises a FEC combination chemotherapy. Exemplary cancers treated by a FEC combination chemotherapy include but are not limited to breast cancers. In some embodiments, the combination chemotherapy comprises a FLAG-IDA combination chemotherapy. Exemplary cancers treated by a FLAF-IDA combination chemotherapy include but are not limited to acute myelogenous leukemias. In some embodiments, the combination chemotherapy comprises a FLO combination chemotherapy. Exemplary cancers treated by a FLO combination chemotherapy include but are not limited to colorectal cancers and gastric cancers. In some embodiments, the combination chemotherapy comprises a FLOX, FOLFIRI, FOLFOX or FOLFOXIRI combination chemotherapy. Exemplary cancers treated by a FLOX, FOLFIRI, FOLFOX or FOLFOXIRI combination chemotherapy include but are not limited to colorectal cancers. In some embodiments, the combination chemotherapy comprises a GEMOX-B combination chemotherapy. Exemplary cancers treated by a GEMOX-B combination chemotherapy include but are not limited to hepatocellular cancers. In some embodiments, the combination chemotherapy comprises a GVD combination chemotherapy. Exemplary cancers treated by a GVD combination chemotherapy include but are not limited to Hodgkin lymphomas. In some embodiments, the combination chemotherapy comprises a Hyper-CVAD combination chemotherapy. Exemplary cancers treated by a hyper-CVAD combination chemotherapy include but are not limited to acute lymphocytic leukemias. In some embodiments, the combination chemotherapy comprises an ICE combination chemotherapy. Exemplary cancers treated by an ICE combination chemotherapy include but are not limited to non-Hodgkin lymphomas. In some embodiments, the combination chemotherapy comprises an ICE-V combination chemotherapy. Exemplary cancers treated by an ICE-V combination chemotherapy include but are not limited to small cell lung cancers. In some embodiments, the combination chemotherapy comprises an IFL, IROX or LV5FU2 combination chemotherapy. Exemplary cancers treated by an IFL, IROX or LV5FU2 combination chemotherapy include but are not limited to colorectal cancers. In some embodiments, the combination chemotherapy comprises an LV5FU-P combination chemotherapy. Exemplary cancers treated by an LV5FU-P combination chemotherapy include but are not limited to biliary cancers. In some embodiments, the combination chemotherapy comprises a MAID combination chemotherapy. Exemplary cancers treated by a MAID combination chemotherapy include but are not limited to sarcomas. In some embodiments, the combination chemotherapy comprises a MFL combination chemotherapy. Exemplary cancers treated by a MFL combination chemotherapy include but are not limited to breast cancers. In some embodiments, the combination chemotherapy comprises a MINE combination chemotherapy. Exemplary cancers treated by a MINE combination chemotherapy include but are not limited to lymphomas. In some embodiments, the combination chemotherapy comprises a MOPP combination chemotherapy. Exemplary cancers treated by a MOPP combination chemotherapy include but are not limited to Hodgkin lymphomas. In some embodiments, the combination chemotherapy comprises a MP combination chemotherapy. Exemplary cancers treated by a MP combination chemotherapy include but are not limited to multiple myelomas. In some embodiments, the combination chemotherapy comprises a MVP combination chemotherapy. Exemplary cancers treated by a MVP combination chemotherapy include but are not limited to lung cancers, mesotheliomas and breast cancers. In some embodiments, the combination chemotherapy comprises a MVAC combination chemotherapy. Exemplary cancers treated by a MVAC combination chemotherapy include but are not limited to bladder cancers. In some embodiments, the combination chemotherapy comprises an OFF combination chemotherapy. Exemplary cancers treated by an OFF combination chemotherapy include but are not limited to pancreatic cancers. In some embodiments, the combination chemotherapy comprises a PAC combination chemotherapy. Exemplary cancers treated by a PAC combination chemotherapy include but are not limited to lymphomas. In some embodiments, the combination chemotherapy comprises a PAD combination chemotherapy. Exemplary cancers treated by a PAD combination chemotherapy include but are not limited to multiple myelomas. In some embodiments, the combination chemotherapy comprises a PCR combination chemotherapy. Exemplary cancers treated by a PCR combination chemotherapy include but are not limited to chronic lymphocytic leukemias. In some embodiments, the combination chemotherapy comprises a PCV combination chemotherapy. Exemplary cancers treated by a PCV combination chemotherapy include but are not limited to brain cancers. In some embodiments, the combination chemotherapy comprises an R-MPV combination chemotherapy. Exemplary cancers treated by an R-MPV combination chemotherapy include but are not limited to central nervous system lymphomas. In some embodiments, the combination chemotherapy comprises an R-GemOx, R-CVP, R-FCM or RICE combination chemotherapy. Exemplary cancers treated by an R-GemOx, R-CVP, R-FCM or RICE combination chemotherapy include but are not limited to non-Hodgkin lymphomas. In some embodiments, the combination chemotherapy comprises a TAC, TC, TCH or TPC combination chemotherapy. Exemplary cancers treated by a TAC, TC, TCH or TPC combination chemotherapy include but are not limited to breast cancers. In some embodiments, the combination chemotherapy comprises a TIP or VIP combination chemotherapy. Exemplary cancers treated by a TIP or VIP combination chemotherapy include but are not limited to testicular cancers. In some embodiments, the combination chemotherapy comprises a TPF combination chemotherapy. Exemplary cancers treated by a TPF combination chemotherapy include but are not limited to head and neck cancers. In some embodiments, the combination chemotherapy comprises a VAD combination chemotherapy. Exemplary cancers treated by a VAD combination chemotherapy include but are not limited to multiple myelomas. In some embodiments, the combination chemotherapy comprises a VMP or VMPT combination chemotherapy. Exemplary cancers treated by a VMP or VMPT combination chemotherapy include but are not limited to multiple myelomas. In some embodiments, the combination chemotherapy comprises a XELIRI or XELOX combination chemotherapy. Exemplary cancers treated by a XELIRI or XELOX combination chemotherapy include but are not limited to colorectal cancers.

Therapeutic or chemotherapeutic agents of the disclosure (including a first and/or one or more second agents) may be administered by any appropriate route including, but not limited to, enteral routes, and parenteral routes, e.g., oral routes, intravenous routes, intramuscular routes, and direct absorption through mucous membrane tissues. The therapeutic agents can be administered by the same route or by different routes.

Therapeutic or chemotherapeutic agents of the disclosure (including a first and/or one or more second agents) may be administered simultaneously with the composition comprising mebendazole. In some embodiments, the additional therapeutic agent and the composition comprising mebendazole are in the same composition. For, the additional therapeutic agent and mebendazole are formulated in the same nanoparticle. Alternatively, or in addition, the additional therapeutic agent or the composition comprising mebendazole is formulated in a nanoparticle. Alternatively, or in addition, the additional therapeutic agent and the composition comprising mebendazole is formulated in different nanoparticles in the same composition.

In some embodiments, the additional therapeutic agent and the composition comprising mebendazole are in the different compositions which are administered simultaneously. This administration can be by any route of administration. For example, the composition comprising mebendazole and the additional therapeutic agent are both administered orally. Alternatively, the composition comprising mebendazole is administered orally and the additional therapeutic agent is administered intravenously. Alternatively, the composition comprising mebendazole is administered orally and the additional therapeutic agent is a combination therapy that is administered orally and intravenously.

Therapeutic or chemotherapeutic agents of the disclosure (including a first and/or one or more second agents) may be administered sequentially with the composition comprising mebendazole. As used herein, the term “sequential administration” refers to administration in a series of ordered steps. For example, the composition comprising mebendazole is administered first, and the additional therapeutic agent is administered second. Alternatively, the composition comprising mebendazole is administered second, and the additional therapeutic agent is administered first. In some embodiments, the sequential administration is repeated, for example in a repeating series. Sequential administration can be separated by any length of time, for example 1 hour, 2 hours, 3 hours, 6 hours, 12 hours, 1 day, 2 days, 3 days, 1, week or 1 month.

Administration of the additional therapeutic or chemotherapeutic agent(s) can be in temporal proximity. Sequential administration of the additional therapeutic or chemotherapeutic agent(s) can be in temporal proximity. As used herein, the term “temporal proximity” refers to administrations that occur close together in time. For example, administrations separated by less than a minute, less than 5 minutes, less than 15 minutes, less than 30 minutes, less than 1 hour, less than 3 hours, less than 6 hours, less than 8 hours, less than 12 hours, less than 1 day, less than 2 days, less than 3 days or less than 1 week occur in temporal proximity.

In some embodiments of the methods of treating cancer of the disclosure comprising administering a therapeutically effective amount of a composition comprising mebendazole, the treatment further comprises administering a cyclin dependent kinase (CDK) inhibitor. CDKs, together with cyclins, control the progression of the cell through the cell cycle. As cells divide, they pass through a number of checkpoints that divide the cell cycle into phases called growth 0 (no division), growth 1 (G1), synthesis (S, when DNA replication occurs), growth 2 (G2) and mitosis (M). Cyclin-CDK complexes regulate the progression of the cell through the phases of the cell cycle. Cyclins bind to their cognate CDKs with a degree of specificity, activating the CDK upon binding and allowing the CDK to phosphorylate targets and regulate the cell cycle. For example, CDK4 and CDK6 (CDK4/6) bind to D type cyclins. Upon binding to a D type cyclin, CDK4/6 phosphorylate RB, which leads to the induction of genes necessary to regulate G1 cellular activity. CDK4 binds to cyclins D1, D2, and D3 to regulate G1. CDK6 binds to cyclins D1, D2, and D3 to regulate G1. CDK2 binds to cyclin E to regulate the G1/S transition. CDK2 binds to cyclin A to regulate S phase. CDK1 cyclin A to regulate the G2/M transition. CDK1 binds to cyclin B to regulate mitosis. CDK9 binds to cyclin T to regulate gene expression. Because of their role in the cell cycle, and hence cell proliferation, CDKs and cyclins are often mis-regulated in cancer cells. For example, CDKs and cyclins can be overexpressed, underexpressed or expressed at the wrong time in the cell cycle in cancer cells. Overexpression of cyclins is associated with some cancers. CDK inhibitors, by inhibiting CDK dependent cell cycle progression, can block the proliferation of cancer cells. Exemplary but non-limiting examples of CDKs targeted by a CDK inhibitor of the disclosure comprise CDK1, CDK2, CDK4, CDK6 and CDK9. Exemplary but non-limiting CDK inhibitors comprise Abemaciclib (Verzenio), Palbociclib (Ibrance), Ribociclib (Kisqali) and analogs or derivatives thereof. Cyclin inhibitors, by inhibiting cyclin dependent CDK activation and CDK dependent cell cycle progression, can also block the proliferation of cancer cells. Exemplary but non-limiting examples of cyclins targeted by a cyclin inhibitor of the disclosure comprise cyclins D1, D2, D3, B, A, E, and T.

In some embodiments of the methods of treating cancer of the disclosure comprising administering a therapeutically effective amount of a composition comprising mebendazole, the treatment further comprises administering an mTOR inhibitor. Mechanistic target or rapamycin kinase (MTOR or mTOR) is a serine threonine kinase that belongs to the family of phosphatidylinositol-3 kinase (PI3K) related kinases. The mTOR pathway plays an important role in regulating cellular growth, metabolism, proliferation and apoptosis. For example, mTOR is downstream of PI3K and Protein kinase B (PKB, also known as AKT), a signaling pathway involved in regulating normal cellular processes such as cell cycle progression, growth, proliferation and survival. Aberrant activation of the PI3K/Akt/mTOR pathway is associated with many human cancers. Activation of this pathway in cancer cells occurs, for example, through the overexpression of genes or proteins in the pathway, or through the loss of function of inhibitors of the pathway. For example, loss of function of the PTEN tumor suppressor, which negatively regulates the PI3K/AKT, leads to upregulation of PI3K/AKT/mTOR pathway in cancer cells. mTOR functions through two distinct complexes, mTORC1 and mTORC2, which interact with each other and elements of other signaling pathways. Because of its role in cell proliferation, growth and survival, inhibiting the mTOR pathway can inhibit the proliferation and growth of cancer cells. Inhibitors of mTOR can inhibit the function of the mTOR protein, or act on other members of the mTOR signaling pathway to reduce the activity of the mTOR pathway. For example, mTOR inhibitors can inhibit the function of members of the mTORC1 or mTORC2 complexes, or inhibit the function of proteins upstream or downstream of mTOR in the mTOR signaling pathway. Exemplary, but non-limiting mTOR inhibitors comprise rapamycin, temsirolimus, everolimus, ridoforolimus and analogs or derivatives thereof. All inhibitors or mTOR are considered within the scope of the invention.

The methods of, or compounds or medicaments for use in combination therapy featured in the disclosure may result in a synergistic effect, wherein the effect of a combination of therapeutic agents (e.g. mebendazole or a pharmaceutically acceptable salt thereof, and one or more second anti-cancer agents) is greater than the sum of the effects resulting from administration of any of the therapeutic agents as single agents. A synergistic effect may also be an effect that cannot be achieved by administration of any of the therapeutic agents as single agents. The synergistic effect may include, but is not limited to, an effect of treating cancer, e.g., by reducing tumor size, reducing the number or frequency of malignant cells in a subject or a sample obtained from a subject, inhibiting tumor growth, inhibiting growth, survival, or proliferation of malignant cells, or increasing survival of the subject. The synergistic effect may also include reducing cancer cell viability, inducing cancer cell death, and inhibiting or delaying cancer cell growth.

Methods of measuring synergy are well known in the art. For example, synergy can be measured using the Chou-Talalay method described in Chou and Talalay (Cancer Res. 2010 Jan. 15; 70(2):440-6. doi: 10.1158/0008-5472.CAN-09-1947. Epub 2010 Jan. 12), the contents of which are herein incorporated by reference in their entirety. The Chou-Talalay method provides an objective, quantitative measure of synergy between combinations of two or more agents. In brief, the Chou-Talalay method measures the effect of a combination of agents on cells, for example on cell viability, at different concentrations of the combination of agents. The different concentrations are preferably at a constant ratio. This data is used to generate a combination index (CI) plot and determine CI values for each of the different concentrations of the combination of agents. When the effects of two agents are additive, the CI is equal to 1. When the effects of two agents are synergistic, the CI is less than 1, preferably less than 0.9. When the effects of two agents are antagonistic, the CI is greater than 1.

In some embodiments, the CI of MBZ and an additional cancer therapy, e.g. an additional therapeutic agent or chemotherapeutic agent, is measured in vitro in a cancer cell line isolated or derived from a rare cancer.

A combination of MBZ and an additional cancer therapy at a particular concentration is synergistic if the CI is less than 0.9. A combination of MBZ and an additional cancer therapy at a particular concentration is strongly synergistic if the CI is less than 0.5, less than 0.4, less than less than 0.3, less than 0.2 or less than 0.1.

A combination of MBZ and an additional cancer therapy is synergistic for an indicated cancer if at least 3 different concentrations of the combination of MBZ and the additional cancer therapy have a CI of less than 0.9 when assayed in vitro in a cancer cell line isolated or derived from the indicated cancer.

A combination of MBZ and an additional cancer therapy is synergistic for an indicated cancer if at least 3 different concentrations of the combination of MBZ and the additional cancer therapy have a CI of less than 0.9 when assayed in vitro in at least one cancer cell line isolated or derived from the indicated cancer.

A combination of MBZ and an additional cancer therapy is strongly synergistic for an indicated cancer if at least 3 different concentrations of the combination of MBZ and the additional cancer therapy have a CI of less than 0.9 when assayed in vitro in more than one cancer cell line isolated or derived from the indicated cancer.

When two agents act synergistically in combination, the therapeutically effective dose of each agent in the combination is typically less than the effective dose of either agent acting as monotherapy. Methods of treatment comprising a synergistic combination of two agents, for example a synergistic combination of MBZ and an additional cancer therapy, therefore typically use lower dosages of one or both agents in the synergistic combination. These lower doses reduce toxicity and harmful side effects. Thus, synergistic combinations of MBZ and an additional cancer therapy provide superior safety and efficacy when compared to monotherapies, or combinations of therapies that do not act synergistically.

In some embodiments, a synergistic amount of the composition comprising mebendazole and the one or more additional therapeutic agents is a specific concentration of the composition comprising mebendazole and the one or more additional therapeutic agents. In some embodiments, a synergistic amount of the composition comprising mebendazole and the one or more additional therapeutic agents is a specific ratio of the composition comprising mebendazole to the one or more additional therapeutic agents. Methods for determining synergistic concentrations and ratios of the agents in a synergistic combination will be readily apparent to one of ordinary skill in the art.

The synergistic effect of the composition comprising mebendazole and the one or more additional therapeutic agents may include, but is not limited to an effect of treating cancer, e.g., by reducing tumor size, reducing the number or frequency of malignant cells in a subject or a sample obtained from a subject, inhibiting tumor growth, inhibiting growth, survival, or proliferation of malignant cells, or increasing survival of the subject. The synergistic effect effect may also include reducing cancer cell viability, inducing cancer cell death, and inhibiting or delaying cancer cell growth.

In some embodiments of the methods of the disclosure, the effect of mebendazole or a pharmaceutically acceptable salt thereof and the one or more additional therapeutic agents may be additive. As used herein, “additive” refers to an effect on a cancer of a subject that is equal to, and not greater than sum of the effects of mebendazole and the additional therapeutic agent were each administered to a subject alone. The additive effect may include, but is not limited to an effect of treating cancer, e.g., by reducing tumor size, reducing the number or frequency of malignant cells in a subject or a sample obtained from a subject, inhibiting tumor growth, inhibiting growth, survival, or proliferation of malignant cells, or increasing survival of the subject. The additive effect may also include reducing cancer cell viability, inducing cancer cell death, and inhibiting or delaying cancer cell growth.

In some embodiments, “combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Therapeutic agents may also be administered in alternation.

In certain aspects of the invention “combination therapy” or “combinational therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In further aspects, a composition of the disclosure, or a pharmaceutically acceptable salt, solvate, analog or derivative thereof, may be administered in combination with radiation therapy. Radiation therapy can also be administered in combination with a composition of the disclosure and another chemotherapeutic agent described herein as part of a multiple agent therapy.

In some embodiments of the methods of the treating cancer of the disclosure, in addition to administering a therapeutically effective amount of the composition comprising mebendazole, and, optionally, a second therapeutic agent or combination of therapeutic agents, the treatment further comprises an immunotherapy. In some embodiments, the immunotherapy comprises a CAR-T therapy. In some embodiments, the immunotherapy comprises an immune checkpoint modulator. In some embodiments, the immune checkpoint modulator comprises an antibody that binds to a component of or otherwise affects the cell cycle checkpoint pathway. In some embodiments, the immune checkpoint modulator modulates a T cell response to cancer. In some embodiments, the immune checkpoint modulator comprises a PD-1 or PD-L 1 checkpoint inhibitor. In some embodiments, the immune checkpoint modulator comprises a CLTA-4 checkpoint inhibitor. Exemplary but non-limiting immunotherapies comprise administering to the subject a therapeutically effective amount of a therapeutic antibody or antibody-drug conjugate. Exemplary, but non-limiting therapeutic antibodies and antibody-drug conjugates which may, in some embodiments, be administered in combination with the composition comprising mebendazole of the disclosure are listed in Table 4.

TABLE 4
Therapeutic Antibodies

| generic name | brand name ® |
|---|---|
| Ado-trastuzumab Emtansine | Kadcycla |
| Alemtuzumab | Campath |
| Atezolizumab | Tecentriq |
| Avelumab | Bavencio |
| Bevacizumab | Avastin |
| Blinatumomab | Blincyto |
| Brentuximab Vedotin | Adcetris |
| Catumaxumab | Proxinium |
| Cetuximab | Erbitux |
| Daratumumab | Darzalex |
| Denosumab | Xgeva |
| Dinutuximab | Unituxin |
| Durvalumab | Imfinzi |
| Elotuzumab | Empliciti |
| Gemtuzumab ozogamicin | Mylotarg |
| Ibritumumab Tiuxetan | Zevalin |
| Ipilimumab | Yervoy |
| Inotuzumab ozogamicin | Besponsa |
| Mogamulizumab | Poteligeo |
| Necitumumab | Portrazza |
| Nivolumab | Opdivo |
| Obinutuzumab | Gazyva |
| Ocrelizumab | Ocrevus |
| Ofatumab | Arzerra |
| Olaratumab | Lartruvo |
| Panitumumab | Vectibix |
| Pembrolizumab | Keytruda |
| Pertuzumab | Perjeta |
| Ramucirumab | Cyramza |
| Rituximab | Rituxan |
| Tositumomab | Bexxar |
| Trastuzumab | Herceptin |
| Zevalin | Ibritumomab tiuxetan |

Exemplary PD-L antibodies include, but are not limited to Atezolizumab, Avelumab and Durvalumab. Exemplary PD-1 antibodies include, but are not limited to Pembrolizumab, Nivolumab and Cemiplimab. Exemplary CTLA-4 antibodies include, but are not limited to Ipilimumab.

As used herein, the “term cell cycle checkpoint” refers to one of several points in the eukaryotic cell cycle at which progression of the cell to the next stage of the cell cycle can be halted under unfavorable conditions. Exemplary, but non-limiting unfavorable conditions comprise improper mitotic spindle formation, excessive levels of DNA damage, and problems with DNA replication.

A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c. Alternatively, or in addition, a cancer can be staged according to the TNM staging system, which divides most types of cancers into 4 stages. Stage 1 usually means that a cancer is relatively small and contained within the organ of origin. Stage 2 cancers have usually not started to spread into surround tissues, but that the tumor is larger than stage 1. In some embodiments, stage 2 means that the cancer has spread into the lymph nodes close to the tumor. Stage 3 cancers are usually larger, and have started to spread into surrounding tissues and lymph nodes. Stage 4, or metastatic cancers, are typically cancers that have spread from the point of origin to other organ(s) in the body.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compound of the present invention, or a pharmaceutically acceptable salt, polymorph, solvate, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present invention, or a pharmaceutically acceptable salt, polymorph or solvate thereof, is more effective than combination therapy in inducing a desired biological effect.

As used herein, “treating” or “treat” describes the management and care of a subject for the purpose of combating a disease, condition, or disorder and includes the administration of a composition of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

As used herein, the term “aggressive” indicates a cancer that can grow, form or spread quickly. Cancers termed aggressive may be susceptible to treatment, or they may resist treatment. An aggressive cancer can comprise any sort of cancer. Alternatively, or in addition, the term “aggressive” may describe a cancer that requires a more severe or intense than the usual form of treatment for that cancer.

As used herein, the term “refractory” describes a cancer that does not respond to an attempted form of treatment. Refractory cancers can also be termed resistant cancers.

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, polymorph, solvate, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, polymorph, solvate, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

### Pharmaceutical Compositions

A “pharmaceutical composition” is a formulation comprising mebendazole in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Mebendazole can be administered to a subject in many of the well-known methods currently used for therapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent a cancer in a subject, or to exhibit a detectable therapeutic or inhibitory effect on said cancer in a subject. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. In some embodiments, a standard xenograft or patient derived xenograft mouse model can be used to determine the effectiveness of mebendazole on a cancer of the disclosure. Such information can then be used to determine useful doses and routes for administration in humans. Therapeutic/prophylactic efficacy and toxicity may be determined by standard pharmaceutical procedures in cell cultures or experimental animals, e.g., the maximum tolerated dose and no observable adverse effect dose. Pharmaceutical compositions that exhibit large therapeutic windows are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing mebendazole may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required nanoparticle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by intramuscular, subcutaneous, transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages may vary depending on the age and size of the subject and the type and severity of the cancer. In some embodiments, the dosage comprises at least 30 mg, at least 50 mg, at least 100 mg, at least 200 mg, at least 300 mg, at least 400 mg, at least 500 mg, at least 600 mg, at least 700 mg, at least 800 mg at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg or at least 2000 mg of mebendazole per day.

In some embodiments, the composition comprising mebendazole is administered parenterally. In some embodiments, the parenteral administration comprises intramuscular, subcutaneous or intravenous administration. In some embodiments, the administration occurs once a day. In some embodiments, the administration occurs twice a day. In some embodiments, the administration occurs three times a day. In some embodiments, the administration occurs four or more times a day. In some embodiments, the subject is administered a composition comprising a therapeutically effective amount of the composition comprising mebendazole, for at least a week, at least a month, at least 2 months, at least 3 months, at least 4 months, at least 5 months, at least 6 months, at least 1 year, at least 2 years, at least 3 years or until the cancer is alleviated.

In some embodiments, the composition comprising mebendazole is administered orally. In some embodiments, the oral administration comprises administration with food. In some embodiments, the administration occurs once a day. In some embodiments, the administration occurs twice a day. In some embodiments, the administration occurs three times a day. In some embodiments, the administration occurs four or more times a day. In some embodiments, the subject is administered a composition comprising a therapeutically effective amount of the composition comprising mebendazole, for at least a week, at least a month, at least 2 months, at least 3 months, at least 4 months, at least 5 months, at least 6 months, at least 1 year, at least 2 years, at least 3 years or until the cancer is alleviated.

In some embodiments, the composition comprising mebendazole is administered daily, every day, without a holiday. In some embodiments, the composition comprising mebendazole is administered with a holiday. In some embodiments, this holiday is once a week. In some embodiments, this holiday is twice a week. In some embodiments, this holiday is once every other week. In some embodiments, this holiday is once a month. In some embodiments, this holiday is determined by the effectiveness of the mebendazole in alleviating a sign or a symptom of the cancer, and/or how well the subject with the cancer tolerates the administration of the composition comprising mebendazole.

In some embodiments, the composition comprising mebendazole is administered simultaneously with an additional cancer therapy. In some embodiments, the composition comprising mebendazole is administered before an additional cancer therapy. In some embodiments, the composition comprising mebendazole is administered after an additional cancer therapy. In some embodiments, the composition comprising mebendazole and the additional cancer therapy are administered in alternation. In some embodiments, this additional cancer therapy comprises an additional chemotherapy.

An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

Mebendazole is capable of further forming salts. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc. Exemplary, but non-limiting mebendazole salts of the disclosure comprise comprises a mebendazole hydrochloride salt ((5-benzoyl-1H-benzimidazole-2-yl)-carbamic acid methyl ester hydrochloride, MBZ.HCl), a mebendazole hydrobromide salt, a mebendazole maleate salt, a mebendazole-glutaric acid co-crystal or a mebendazole monomethyl oxalate salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

The mebendazole, or pharmaceutically acceptable salts or solvates thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In some embodiment, the composition comprising mebendazole is administered orally. In some embodiments, the oral administration occurs with food. In some embodiments, the composition comprising mebendazole is administered parenterally. In some embodiments, the parenteral administration comprises intramuscular, subcutaneous or intravenous administration. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19^{th} edition, Mack Publishing Co., Easton, Pa. (1995). In some embodiments, mebendazole, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits and Articles of Manufacture

The invention provides kits comprising any one or more of the compositions described herein, not limited to compositions comprising mebendazole and compositions comprising mebendazole and one or more additional therapeutic or chemotherapeutic agents. The kits are for use in the treatment of cancer.

In some embodiments of the kits of the disclosure, the kit comprises a therapeutically effective amount of a composition comprising mebendazole and instructions for use in the treatment of cancer. In some embodiments, the kit further comprises at least one additional cancer therapeutic agent. The composition comprising mebendazole and the additional cancer therapeutic agent are the same composition, e.g. a single pill or tablet formulated for oral administration, or a single liquid composition in a vial formulated for intravenous administration. Alternatively, the composition comprising mebendazole and the additional cancer therapeutic agent are the different compositions both included in the kit.

In some embodiments of the kits of the disclosure, the therapeutically effective amount of the composition comprising mebendazole comprises a synergistically effective amount of the composition comprising mebendazole. In some embodiments, the composition comprising mebendazole and the at least one additional cancer therapeutic agent exhibit synergy. In some embodiments, the at least one additional cancer therapeutic agent comprises comprises a second chemotherapeutic agent, a combination chemotherapy, a therapeutic antibody, a chimeric antigen receptor T cell (CAR-T) therapy or a combination thereof. In some embodiments, the at least one additional cancer therapeutic agent comprises Cisplatin, Doxorubicin, Etoposide, Cyclophosphamide, 5-FU, Gemcitabine, Oxaliplatin, Irinotecan, Vinorelbine, Dacarbazine, Vincristine, Sorafenib, Paclitaxel, Imatinib, Abemaciclib, Ifosamide or Docetaxel.

In some embodiments of the kits of the disclosure, the mebendazole is formulated in a nanoparticle. In some embodiments, the mebendazole and the at least one additional cancer therapeutic are formulated in a nanoparticle and the nanoparticle is the same nanoparticle. In some embodiments, the nanoparticle comprising mebendazole, and, optionally, one or more additional therapeutic agents comprises a PLGA polymer and an HA targeting agent.

Articles of manufacture include, but are not limited to, instructions for use of the kit in treating cancers, for example rare cancer indications of the disclosure, and vials.

### Enumerated Embodiments

The invention may be defined by reference to the following enumerated, illustrative embodiments:

1. A method of treating a cancer in a subject in need thereof, comprising administering to the subject a therapeutically effective amount of a composition comprising methyl N-(6-benzoyl-1H-benzimidazol-2-yl)carbamate (mebendazole).

2. A composition for use in treating cancer in a subject in need thereof comprising a therapeutically effective amount of a composition comprising mebendazole.

3. A composition for use in the manufacture of a medicament for the prevention or treatment of cancer comprising a therapeutically effective amount of a composition comprising mebendazole.

4. The method or composition for use according to any one of embodiments 1-3, wherein the composition comprising mebendazole comprises a salt.

5. The method or composition for use according to embodiment 4, wherein the mebendazole salt comprises a mebendazole hydrochloride salt, a mebendazole hydrobromide salt, a mebendazole maleate salt, a mebendazole-glutarate salt or a mebendazole monomethyl oxalate salt.

6. The method or composition for use according to any one of embodiments 1-5, wherein the composition comprising mebendazole comprises a crystal form or a polymorph of mebendazole.

7. The method or composition for use according to embodiment 6, wherein the polymorph comprises a polymorph A of mebendazole, a polymorph B of mebendazole, a polymorph C of mebendazole or a combination thereof.

8. The method or composition for use according to embodiment 6, wherein the polymorph comprises crystal polymorph C.

9. The method or composition for use according to any one of embodiments 1-8, wherein the composition comprising mebendazole further comprises a nanoparticle.

10. The method or composition for use according to embodiment 9, wherein the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle.

11. The method or composition for use according to embodiment 10, wherein the polymer-based nanoparticle comprises a multiblock copolymer, a diblock copolymer, a polymeric micelle or a hyperbranched macromolecule.

12. The method or composition for use according to embodiment 10, wherein the polymer-based nanoparticle comprises a multiblock copolymer or a diblock copolymer.

13. The method or composition for use according to embodiment 10, wherein the polymer-based nanoparticle comprises a poly(lactic-co-glycolic acid) PLGA polymer.

14. The method or composition for use according to any one of embodiments 11-13, wherein the polymer-based nanoparticle is pH responsive.

15. The method or composition for use according to any one of embodiments 11-13, wherein the polymer-based nanoparticle further comprises a buffering component.

16. The method or composition for use according to any one of embodiments 10-15, wherein the nanoparticle further comprises a targeting agent.

17. The method or composition for use according to embodiment 16, wherein the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

18. The method or composition for use according to embodiment 16, wherein the targeting agent comprises hyaluronic acid (HA).

19. The method or composition for use according to any one of embodiments 16-18, wherein the targeting agent binds to the surface of a cell of the cancer of the subject.

20. The method or composition for use according to any one of embodiments 1-19, wherein the cancer comprises a colorectal cancer, a gastric cancer, a brain cancer, colon cancer, a breast cancer, a liver cancer, a lung cancer, a pancreatic cancer or a renal cancer.

21. The method or composition for use according to 20, wherein the lung cancer comprises a small cell lung cancer or a non-small cell lung cancer.

22. The method or composition for use according to any one of embodiments 1-21, wherein the cancer is a rare cancer.

23. The method or composition for use according to embodiment 22, wherein the cancer is a blastoma, a sarcoma, a carcinoma, a neuroendocrine cancer, a mesothelioma, a chordoma, a thymic cancer, a gastrointestinal stromal tumor or a pheochromocytoma.

24. The method or composition for use according to embodiment 23, wherein the blastoma comprises a neuroblastoma or a glioblastoma.

25. The method or composition for use according to embodiment 23, wherein the sarcoma comprises an Ewing's sarcoma, a leiomyosarcoma, an angiosarcoma or a rhabdomyosarcoma.

26. The method or composition for use according to embodiment 23, wherein the carcinoma comprises an adenoid cystic carcinoma (ACC), a uterine serous carcinoma, an adrenocortical carcinoma, a gastric carcinoma, a cholangiocarcinoma, a colorectal carcinoma, an esophageal carcinoma, a hepatocellular carcinoma, a pancreatic carcinoma, a small cell lung carcinoma, an ovarian carcinoma or a thymic carcinoma.

27. The method or composition for use according to embodiment 26, wherein the adenoid cystic carcinoma (ACC) comprises a salivary gland cell, a trachea cell, a lacrimal gland cell, a breast cell, a skin cell or a vulval cell.

28. The method or composition for use according to embodiment 23, wherein the thymic cancer comprises a thymoma or a thymic carcinoma.

29. The method or composition for use according to embodiment 23, wherein the neuroendocrine cancer comprises a carcinoid tumor or a thymic cancer.

30. The method or composition for use according to embodiment 29, wherein the carcinoid tumor comprises a small intestine tumor, an appendix tumor, a tumor of the rectum, a tumor of the bronchial system, a brain tumor, colon tumor, a stomach tumor, a pancreatic tumor, a liver tumor, a gallbladder tumor, a bile duct tumor, an ovarian tumor, a testicular tumor, a bladder tumor, a tumor of the prostate gland, a breast tumor, a kidney tumor, a thymic tumor, an eye tumor, an ear tumor or an adrenal tumor.

31. The method or composition for use according to any one of embodiments 1-30 wherein the cancer is a stage 0 or stage 1 (early stage, pre-metastatic) cancer.

32. The method or composition for use according to any one of embodiments 1-30, wherein the cancer is a stage 2 cancer or stage 3 (spread to nearby tissues and lymph nodes) cancer.

33. The method or composition for use according to any one of embodiments 1-30, wherein the cancer is a stage 4 (advanced or metastatic) cancer.

34. The method or composition for use according to any one of embodiments 1-33, wherein the subject is a mammal, a non-human primate or a human.

35. The method or composition for use according to any one of embodiments 1-33, wherein the subject is human.

36. The method or composition for use according to embodiment 35, wherein the human is a male, a female, a child, a baby or a neonate.

37. The method or composition for use according to any one of embodiments 1-36, wherein the composition comprising mebendazole is suitable for systemic, oral or parenteral administration.

38. The method or composition for use according to embodiment 37, wherein the administration comprises at least 30 mg, 50 mg, 100 mg, 200 mg, 300 mg, 400 mg, 500 mg, 600 mg, 700 mg, 800 mg 900 mg, 1000 mg, 1100 mg, 1200 mg, 1300 mg, 1400 mg, 1500 mg, 1600 mg, 1700 mg, 1800 mg, 1900 mg or 2000 mg of mebendazole per day.

39. The method or composition for use according to 37, wherein the oral administration occurs with food.

40. The method or composition for use according to of embodiment 37, wherein the parenteral administration comprises intramuscular, subcutaneous or intravenous administration.

41. The method or composition for use according to any one of embodiments 37-40, wherein the administration occurs once a day, twice a day, three times a day or four or more times a day.

42. The method or composition for use according to any one of embodiments 1-41, wherein the method of treatment or composition for use further comprises an additional cancer treatment.

43. The method or composition for use according to embodiment 42, wherein the additional cancer treatment comprises a surgical procedure to remove at least one tumor of the cancer or at least one dose of a radiation therapy.

44. The method or composition for use according to embodiment 42 or 43, wherein the additional cancer treatment comprises a second chemotherapeutic agent, a combination chemotherapy, a therapeutic antibody, a chimeric antigen receptor T cell (CAR-T) therapy or a combination thereof.

45. The method or composition for use according to embodiment 44, wherein the second chemotherapeutic agent comprises a cell cycle checkpoint inhibitor, a CDK inhibitor, an mTOR inhibitor, an immune checkpoint modulator, an antimitotic agent, a pro-apoptotic agent, a DNA damaging agent or an inhibitor of a DNA damage response pathway.

46. The method or composition for use according to embodiment 45, wherein the CDK inhibitor comprises an inhibitor of CDK4, an inhibitor of CDK6 or an inhibitor of CDK4 and CDK6.

47. The method or composition for use according to embodiment 45, wherein the CDK inhibitor comprises Abemaciclib (Verzenio), Palbociclib (Ibrance) or Ribociclib (Kisqali).

48. The method or composition for use according to embodiment 45, wherein the mTOR inhibitor comprises Rapamycin (Sirolimus), Temsirolimus (Torisel), Everolimus (Afinitor) or Ridaforolimus.

49. The method or composition for use according to embodiment 45, wherein the immune checkpoint modulator comprises Yervoy (Ipilimumab), Opdivo (Nivolumab), Tecentriq (Atezolizumab) or Keytruda (Pembrolizumab).

50. The method or composition for use according to embodiment 44, wherein the second chemotherapeutic agent comprises Abitrexate (Methotrexate), Afinitor (Everolimus), Alimta (PEMETREXED), Alkeran (Melphalan), Aredia (Pamidronate), Arimidex (Anastrozole), Aromasin (Exemestane), Arranon (Nelarabine), Beleodaq (Belinostat), BiCNU (Carmustine), Blenoxane (Bleomycin), Bosulif (Bosutinib), Busulfex (Busulfan), Caprelsa (Vandetanib), Carboplatin, Casodex (Bicalutamide), CeeNU (Lomustine), Cerubidine (Daunorubicin), Cisplatin, Clolar (Clofarabine), Cometriq (Cabozantinib), Cosmegen (Dactinomycin), Cotellic (Cobimetinib), CytosarU (Cytarabine), Cytoxan, Dacarbazine, Dacogen (Decitabine), DaunoXome (Daunorubicin Lipid Complex), Decadron (Dexamethasone), Docetaxel, Doxorubicin, DepoCyt (Cytarabine Lipid Complex), Dexamethasone Intensol (Dexamethasone), Dexpak Taperpak (Dexamethasone), Droxia (Hydroxyurea), Eligard (Leuprolide), Ellence (Epirubicin), Eloxatin (Oxaliplatin), Elspar (Asparaginase), Emcyt (Estramustine), Erivedge (Vismodegib), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), Fludara (Fludarabine), 5-Fluorouracil, Folex (methotrexate), Folotyn (Pralatrexate Injection), FUDR (floxuridine), Gemzar (Gemcitabine), Gilotrif (Afatinib), Gleevec (Imatinib Mesylate), Gliadel (Carmustine), HDAC (high dose Cytarabine), Halaven (Eribulin), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Ifex (Ifosfamide), Imbruvica (Ibrutinib), Inlyta (Axitinib), Intron A alfab (Interferon alfa-2a), Iressa (Gefitinib), Irinotecan, Istodax (Romidepsin), Ixempra (Ixabepilone), Jakafi (Ruxolitinib), Jevtana (Cabazitaxel Injection), Kyprolis (Carfilzomib), Lenvima (Lenvatinib mesylate), Somatuline Depot (Lanreotide acetate), Leukeran (Chlorambucil), Leukine (Sargramostim), Leustatin (Cladribine), Lonsurf (Trifluridine and Tipiracil), Lupron (Leuprolide), Lupron Depot (Leuprolide), Lupron DepotPED (Leuprolide), Lynparza (Olaparib), Lysodren (Mitotane), Matulane (Procarbazine), Xofigo (Radium 223 dichloride), Megace (Megestrol), Mekinist (Trametinib), Mesnex (Mesna), Mesnex (Mesna Injection), Metastron (Strontium-89 Chloride), Mexate (Methotrexate) Mustargen (Mechlorethamine), Mutamycin (Mitomycin), Myleran (Busulfan), Navelbine (Vinorelbine), Neosar (Cyclophosphamide), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Odomzo (Sonidegib), Oncaspar (Pegaspargase), Ontak (Denileukin Diftitox), Paclitaxel, Panretin (Alitretinoin), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), RoferonA alfaa (Interferon alfa-2a), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b), Synribo (Omacetaxin), Tabloid (Thioguanine), Taflinar (Dabrafenib), Tarceva (Erlotinib), Targretin (Bexarotene), Dacarbazine, Temodar (Temozolomide), Tepadina (Thiotepa), Thalomid (Thalidomide), TheraCys BCG (BCG), Thioplex (Thiotepa), TICE BCG (BCG), Toposar (Etoposide), Torisel (Temsirolimus), Yondelis (Trabectedin), Treanda (Bendamustine hydrochloride), Trelstar (Triptorelin), Trexall (Methotrexate), Trisenox (Arsenic trioxide), Tykerb (lapatinib), Valstar (Valrubicin Intravesical), Vantas (Histrelin Implant), Velcade (Bortezomib), Vepesid (Etoposide), Vesanoid (Tretinoin), Vincristine, Vidaza (Azacitidine), Vinblastine, Votrient (Pazopanib), Vumon (Teniposide), Wellcovorin IV (Leucovorin), Xalkori (Crizotinib), Xeloda (Capecitabine), Xtandi (Enzalutamide), Zaltrap (Ziv-aflibercept), Zanosar (Streptozocin), Zelboraf (Vemurafenib), Zoladex (Goserelin), Zolinza (Vorinostat), Zometa (Zoledronic acid), Zortress (Everolimus), Zydelig (Idelalisib), Zykadia (Ceritinib), Zytiga (Abiraterone acetate), Vindesine (Eldesine), Raltitrexed (Tomudex), Lometrexol, Satraplatin, Larotaxel, Alectinib (Alecensa), Ixazomib (Ninlaro), Nilotinib (Tasigna), Osimertinib (Tagrisso), Venetoclax (Venclexta), Ribociclib (Kisqali), Enasidenib (Idhifa), Rucaparib (Rubraca), Niraparib (Zejula), Copanlisib (Aliqopa), Neratinib (Nerlynx), Brigatinib (Alunbrig), Midostaurin (Rydapt), Abemaciclib (Verzenio), Rapamycin (Sirolimus), Temsirolimus (Torisel), Ridaforolimus or a combination thereof.

51. The method or composition for use according to embodiment 44, wherein the second chemotherapeutic agent comprises Paclitaxel, Docetaxel, Vinblastine, Vincristine, Cisplatin, Carboplatin, Oxaliplatin, Doxorubicin, Etoposide, Imatinib, Gemcitabine, Vinorelbine, Ifosamide, Abemaciclib, Sorafenib, Irinotecan, 5-Fluorouracil, Dacarbazine, Trabectedin, Temozolomide, Cyclophosphamide or a combination thereof.

52. The method or composition for use according to embodiment 44, wherein the therapeutic antibody comprises Adcetris (Brentuximab Vedotin), Arzerra (Ofatumumab), Avastin (Bevacizumab), Bexxar (Tositumomab), Bavencio (Avelumab), Blincyto (Blinatumomab), Campath (Alemtuzumab), Cyramza (Ramucirumab), Darzalex (Daratumumab), Empliciti (Elotuzumab), Erbitux (Cetuximab), Gazyva (Obinutuzumab), Imfinzi (Durvalumab), Herceptin (Trastuzumab), Gazyvaro (Obinutuzumab), Kadcyla (Ado-trastuzumab Emtansine), Keytruda (Pembrolizumab), Lartruvo (Olaratumab), Mylotarg (Gemtuzumab Ozogamicin), Ocrevus (Ocrelizumab), Opdivo (Nivolumab), Perjeta (Pertuzumab), Portrazza (Necitumumab), Proxinium (Catumaxomab), Removab (Catumaxomab), Rituxan (Rituximab), Sylvant (Siltuximab), Tecentriq (Atezolizumab), Unituxin (Dinutuximab), Vectibix (Panitumumab), Yervoy (Ipilimumab), Xgeva (Denosumab), Zevalin (Ibritumomab Tiuxetan), Mogamulizumab (Poteligeo) or a combination thereof.

53. The method or composition for use according to embodiment 44, wherein the combination chemotherapy comprises 7+3, ABVD, AC, AD, ADE, ADOC, BEACOPP, BEP, CAF, CAPIRI, CAPOX, CB, CBI, CEF, CEPP, CFAR, CHOP, CIM, CLAG, CLAG-M, CMC, CMF, COI, CVD, CVP, DHAP, DVD, ECF, ECX, EOF, EOX, EP, EPOCH, EPOCH+R, ESHAP, FAMTX, FC, FCR, FEC, FLAG-IDA, FLO, FLOX, FOLFIRI, FOLFOX, FOLFOXIRI, GEMOX-B, GVD, Hyper-CVAD, ICE, ICE-V, IFL, IROX, LV5FU2, LV5FU-P, MAID, MFL, MINE, MOPP, MP, MPV, MVAC, OFF, PAC, PAD, PCR, PCV, R-MPV, R-GemOx, R-CHOP, R-CVP, R-FCM, RICE, TAC, TC, TCH, TIP, TPC, TPF, VAD, VIP, VMP, VMPT, XELIRI or XELOX.

54. The method or composition for use according to any one of embodiments 44-53, wherein the composition comprising mebendazole and the additional cancer treatment are in the same composition.

55. The method or composition for use according to embodiment 54, wherein the composition comprising mebendazole and the additional cancer treatment are formulated in a nanoparticle.

56. The method or composition for use according to any one of embodiments 44-55, wherein the additional cancer treatment and the composition comprising mebendazole are suitable for simultaneous administration.

57. The method or composition for use according to any one of embodiments 44-53, wherein the additional cancer treatment and the composition comprising mebendazole are suitable for sequential administration.

58. The method or composition for use according to any one of embodiments 49-53, wherein the additional cancer treatment and the composition comprising mebendazole are suitable for administration in temporal proximity.

59. The method or composition for use according to any one of embodiments 44-58, wherein the additional cancer treatment and the composition comprising mebendazole exhibit synergy.

60. The method or composition for use according to embodiment 59, wherein the synergy is measured using the Chou-Talalay method in at least one cancer cell line.

61. The method or composition for use according to embodiment 60, wherein the synergy comprises a CI of less than 0.9 when measured at at least three concentrations of the additional cancer treatment and the composition comprising mebendazole in at least one cancer cell line.

62. The method or composition for use according to any one of embodiments 59-61, wherein the composition comprising mebendazole and the additional cancer treatment are each suitable for administration in a syngergistically effective amount.

63. The method or composition for use according to any one of embodiments 1-62, wherein the method or composition for use alleviates a sign or a symptom of the cancer.

64. The method or composition for use according to embodiment 63, wherein the alleviation of the sign or the symptom of the cancer comprises a reduction in size of at least one tumor, a reduction in the volume of at least one tumor, a decrease in the number of tumors, a decrease in the number of metastatic lesions of the cancer, a reduction of the rate of growth of the cancer or a remission of the cancer.

65. A composition comprising a synergistic combination of mebendazole and at least one additional cancer therapeutic agent.

66. The composition of embodiment 65, wherein the synergy is measured using the Chou-Talalay method in at least one cancer cell line.

67. The composition of embodiment 66, wherein the synergy comprises a CI of less than 0.9 when measured at at least three concentrations of the additional cancer treatment and the composition comprising mebendazole in at least one cancer cell line.

68. The composition of any one of embodiments 65-67, wherein the mebendazole comprises a salt.

69. The composition of embodiment 68, wherein the mebendazole salt comprises a mebendazole hydrochloride salt, a mebendazole hydrobromide salt, a mebendazole maleate salt, a mebendazole-glutarate salt or a mebendazole monomethyl oxalate salt.

70. The composition of any one of embodiments 65-67, wherein the mebendazole comprises a crystal form or a polymorph of mebendazole.

71. The composition of embodiment 70, wherein the polymorph comprises a polymorph A of mebendazole, a polymorph B of mebendazole, a polymorph C of mebendazole or a combination thereof.

72. The composition of embodiment 70, wherein the polymorph comprises crystal polymorph C.

73. The composition of any one of embodiments 65-72, wherein the at least one additional cancer therapeutic agent comprises Cisplatin, Doxorubicin, Etoposide, Cyclophosphamide, 5-FU, Gemcitabine, Oxaliplatin, Irinotecan, Vinorelbine, Dacarbazine, Vincristine, Sorafenib, Paclitaxel, Imatinib, Abemaciclib, Ifosamide or Docetaxel.

74. The composition of any one of embodiments 65-73, wherein the mebendazole is formulated in a nanoparticle.

75. The composition of any one of embodiments 65-73, wherein the mebendazole and the at least one additional cancer therapeutic agent are formulated in a nanoparticle.

76. The composition of embodiment 74 or 75, wherein the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle.

77. The composition of embodiment 76, wherein the polymer-based nanoparticle comprises a multiblock copolymer, a diblock copolymer, a polymeric micelle or a hyperbranched macromolecule.

78. The composition of embodiment 76, wherein the polymer-based nanoparticle comprises a multiblock copolymer a diblock copolymer.

79. The composition of embodiment 76, wherein the polymer-based nanoparticle comprises a poly(lactic-co-glycolic acid) PLGA polymer.

80. The composition of any one of embodiments 76-79, wherein the polymer-based nanoparticle is pH responsive.

81. The composition of any one of embodiments 76-80, wherein the polymer-based nanoparticle further comprises a buffering component.

82. The composition of any one of embodiments 76-81, wherein the nanoparticle further comprises a targeting agent.

83. The composition of embodiment 82, wherein the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

84. The composition of embodiment 82, wherein the targeting agent comprises hyaluronic acid (HA).

85. The composition of any one of embodiments 82-84, wherein the targeting agent binds to the surface of a cell of the cancer of the subject.

86. A combinational therapy for treating cancer, comprising administering a therapeutically effective amount of the composition of any one of 64-85 to a subject in need thereof.

87. A combinational therapy for treating cancer, comprising administering a synergistically effective amount of the composition of any one of 64-85 to a subject in need thereof.

88. A composition for use in a combinational therapy to treat cancer, comprising a therapeutically effective amount of the composition comprising mebendazole of any one of embodiments 1-41.

89. The composition according to embodiment 88 for use in a combinational therapy, wherein the combinational therapy comprises administering one or more additional cancer therapies to the subject.

90. A kit comprising the composition of any one of embodiments 64-85 and instructions for use in the treatment of cancer.

91. A kit, comprising a therapeutically effective amount of a composition comprising mebendazole and instructions for use in the treatment of cancer.

92. The kit of embodiment 91, further comprising at least one additional cancer therapeutic agent.

93. The kit of embodiment 92, wherein the therapeutically effective amount of the composition comprising mebendazole comprises a synergistically effective amount of the composition comprising mebendazole.

94. The kit of embodiment 92 or 93, wherein the composition comprising mebendazole and the at least one additional cancer therapeutic agent exhibit synergy.

95. The kit of any one of embodiments 92-94, wherein the at least one additional cancer therapeutic agent comprises a second chemotherapeutic agent, a combination chemotherapy, a therapeutic antibody, a chimeric antigen receptor T cell (CAR-T) therapy or a combination thereof.

96. The kit of any one of embodiments 92-94, wherein the at least one additional cancer therapeutic agent comprises Cisplatin, Doxorubicin, Etoposide, Cyclophosphamide, 5-FU, Gemcitabine, Oxaliplatin, Irinotecan, Vinorelbine, Dacarbazine, Vincristine, Sorafenib, Paclitaxel, Imatinib, Abemaciclib, Ifosamide or Docetaxel.

97. The kit of any one of embodiments 91-96, wherein the mebendazole is formulated in a nanoparticle.

98. The kit of any one of embodiments 91-96, wherein the mebendazole and the at least one additional cancer therapeutic agent are formulated in a nanoparticle.

99. The kit of embodiment 97 or 98, wherein the nanoparticle comprises a PLGA polymer and an HA targeting agent.

### EXAMPLES

To better understand the invention, examples are provided below. These examples are illustrative only and are not intended to be limiting.

### Example 1: Effective Mebendazole on Cancer Cell Lines

The effect of mebendazole administration on cell lines derived from exemplary cancers was tested. One approach to determining the effectiveness of mebendazole in the treatment of cancer comprises in vitro testing. In this approach, cancer cell lines representative of the cancers of the disclosure are cultured in vitro according to standard techniques (see, for example, Human Cell Culture Protocols, Third Edition, R. Mitry and R. D. Hughes, Editors, Humana Press, 2012), and increasing concentrations of mebendazole are administered to determine the IC₅₀ value. As used herein, the term “IC₅₀ value” refers to the concentration of a compound (for example mebendazole) wherein the response to that compound (such as inhibited cellular viability) is reduced by half. The IC₅₀ is thus a measure of the effectiveness of a compound in inhibiting a biological process. In this model, cancerous cell lines representative of the various cancers of the disclosures were cultured, treated with mebendazole in concentrations ranging, typically, from 0.0001 to 10 μM, and the IC₅₀ value was calculated after 24, 48 or 72 hours to determine the effectiveness of mebendazole in killing the cancer cells.

Repurposing of drugs for use in oncology is of increasing interest to promote the rapid development of new therapies and address the significant unmet medical need that remains for rare cancers. The FDA-approved anti-helminthic drug mebendazole (MBZ) has been identified as an agent that can affect several cancer relevant pathways involved in tumor growth and metastasis. Anti-tumor effects of MBZ include G2/M cell cycle arrest leading to apoptosis, and induction of MYB degradation by the proteasome.

Based on this biology, several rare cancer tumor cell lines including neuroblastoma, rhabdomyosarcoma and Ewing sarcoma were analyzed for sensitivity to MBZ treatment in vitro. Cultured cells were exposed to increasing concentrations of MBZ and viability was measured after 72 hours unless otherwise indicated. In all cases, including those described below, the viable cells were determined using the CellTiter Glo® kit and an IC₅₀ curve was generated using the PRISM software. Treatment with MBZ resulted in a significant decrease in cell viability with IC₅₀s ranging from 0.05-1 μM suggesting that MBZ may represent a potent anti-cancer agent for several rare cancer indications.

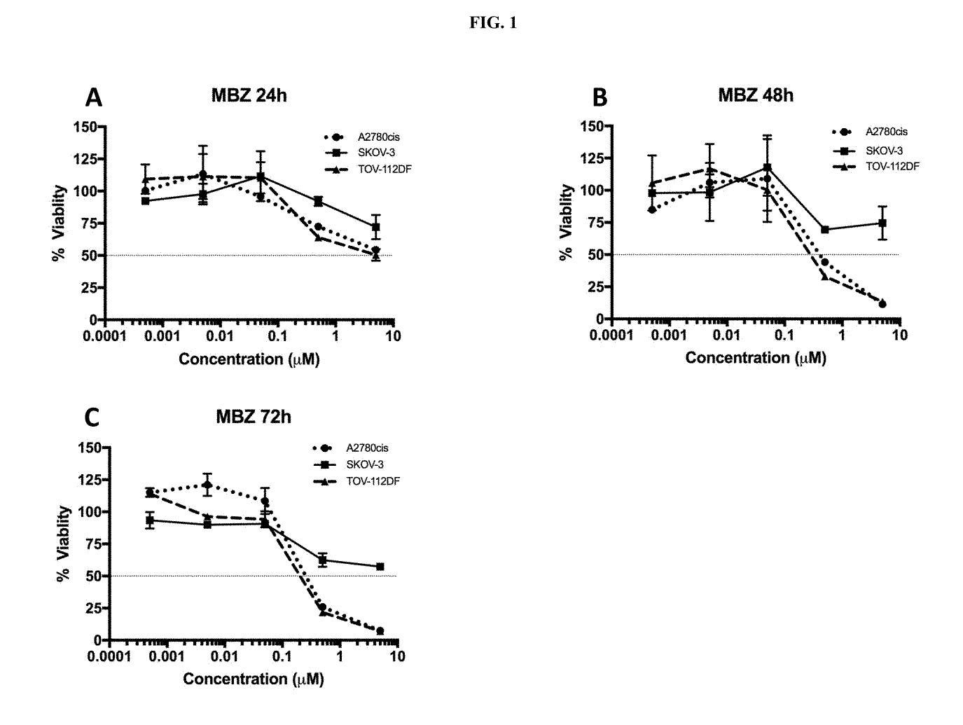

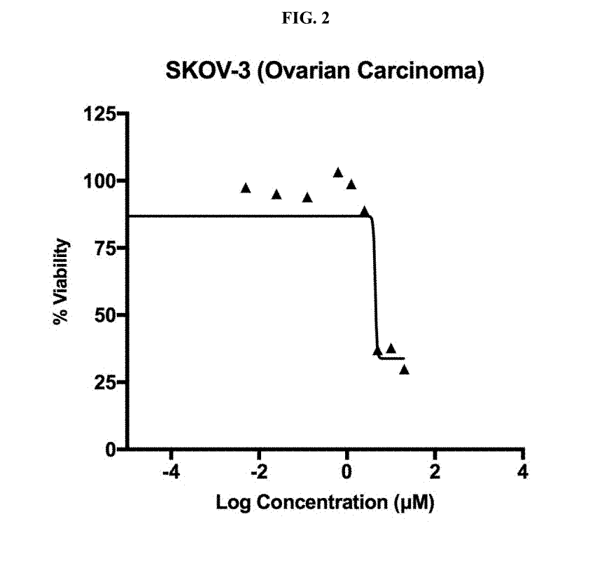

FIGS. 1 and 2 show the effect of mebendazole on 3 ovarian cancer derived cell lines, A2780cis, SKOV-3 and TOV-112D at 24 hours, 48 hours and 72 hours. In FIG. 1, the MBZ concentration (in μM) is shown on the X-axis from 0.0001 to 10 increasing by powers of 10. Percent cell viability is shown on the Y-axis, from 0 to 150 in units of 25. A2780cis cell data are represented by filled circles and a dotted line. SKOV-3 cell data are represented by squares and a solid line. TOV-112D cell data are represented by triangles and a dashed line. At 72 hours the IC₅₀ value for A2780cis and TOV-112D was 0.25 μM and 0.2 μM respectively. In FIG. 2, the ovarian carcinoma cell line SKOV-3 was treated with ten serial dilutions of MBZ (0-50 μM) for 72 hours. In FIG. 2, on the X-axis, the log concentration of mebendazole in μM, indicated from −4 to 4 in units of 2. In FIG. 2, on the Y-axis, percent viability is shown, from 0 to 125 in units of 25. The IC₅₀ for the SKOV-3 cell line following treatment of MBZ was calculated to be ˜4.4 μM.

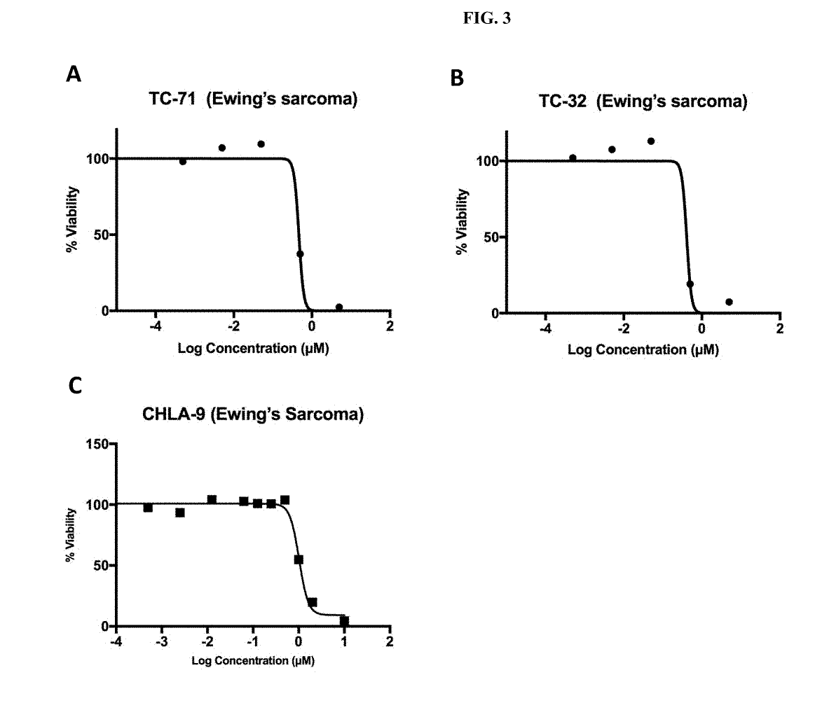

The Ewing's sarcoma cell lines TC-71, TC-32, and CHLA-9 were treated with ten serial dilutions of MBZ for 72 hours. TC-71 (FIG. 3A) and TC-32 (FIG. 3B) were treated with a concentration range of mebendazole ranging from 0-5 μM. CHLA-9 (FIG. 3C) was treated with a concentration range of mebendazole ranging from 0-10 μM. The IC₅₀ for the TC-71 cell line following treatment of MBZ was calculated to be 0.47 μM. The IC₅₀ for the TC-32 cell line following treatment of MBZ was calculated to be 0.41 μM. The IC₅₀ for the CHLA-9 cell line following treatment of MBZ was calculated to be 1.02 μM.

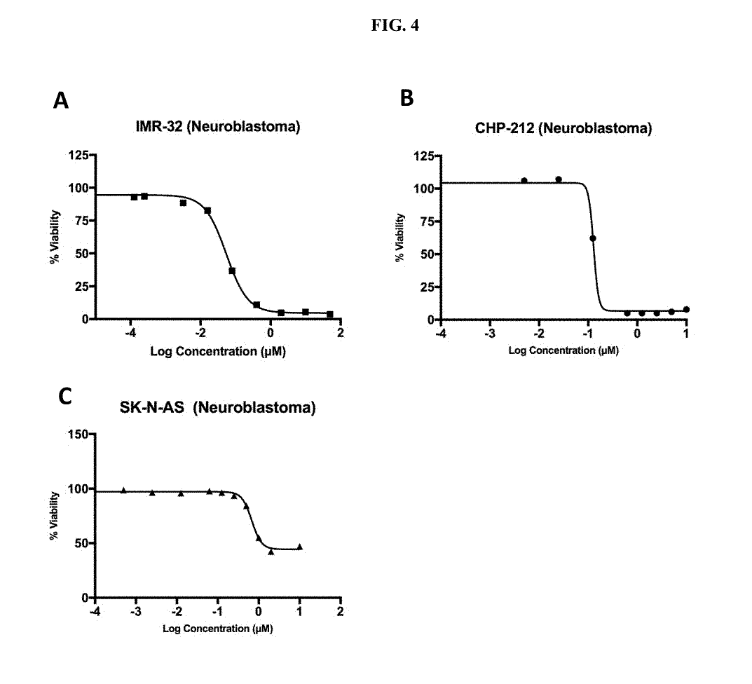

The neuroblastoma cell lines IMR-32, CHP-212, and SK-N-AS were treated with ten serial dilutions of mebendazole for 72 hours. IMR-32 (FIG. 4A) was treated with a concentration range from 0-50 μM. CHP-212 (FIG. 4B) was treated with a concentration range of mebendazole from 0-20 μM. SK-N-AS (FIG. 4C) was treated with a concentration range of mebendazole from 0-10 μM. The IC₅₀ for the IMR-32 cell line following treatment of MBZ was calculated to be 0.05 μM. The IC₅₀ for the CHP-212 cell line following treatment of MBZ was calculated to be 0.13 μM. The IC₅₀ for the SK-N-AS cell line following treatment of MBZ was calculated to be 0.68 μM.

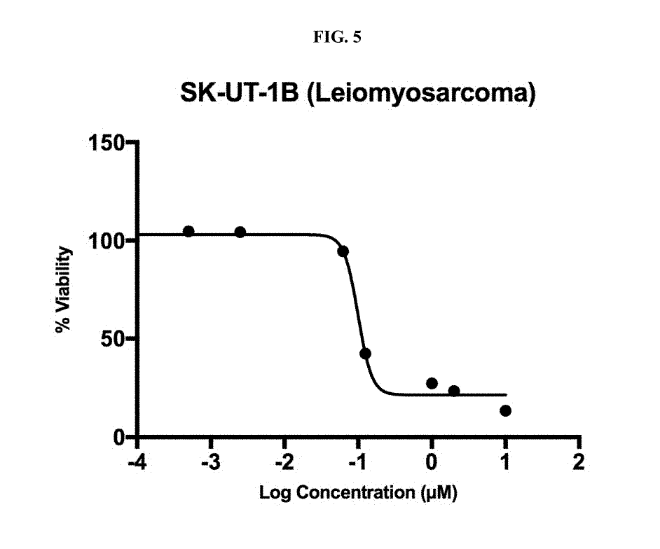

The leiomyosarcoma cell line SK-UT-1B cell line (FIG. 5) was treated with ten serial dilutions of MBZ (0-10 μM) for 72 hours. The IC₅₀ for the SK-UT-1B cell line following treatment of MBZ was calculated to be 0.1 μM.

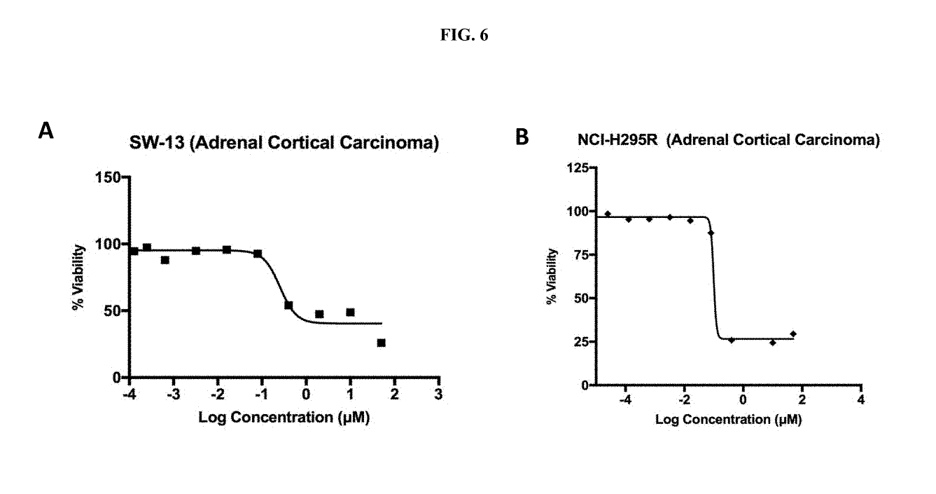

The adrenal cortical carcinoma (ACC) cell lines SW-13 and NCI-H295R were treated with ten serial dilutions of MBZ for 72 hours (FIG. 6). In FIG. 6A, the mebendazole concentration range for SW13 treatment was 0-20 μM. In FIG. 6B, the mebendazole concentration range for NCI-H295R treatment was 0-50 μM. The IC₅₀ for the SW-13 cell line following treatment of MBZ was calculated to be 0.26 μM. The IC₅₀ for the NCI-H295R cell line following treatment of MBZ was calculated to be 0.1 μM.

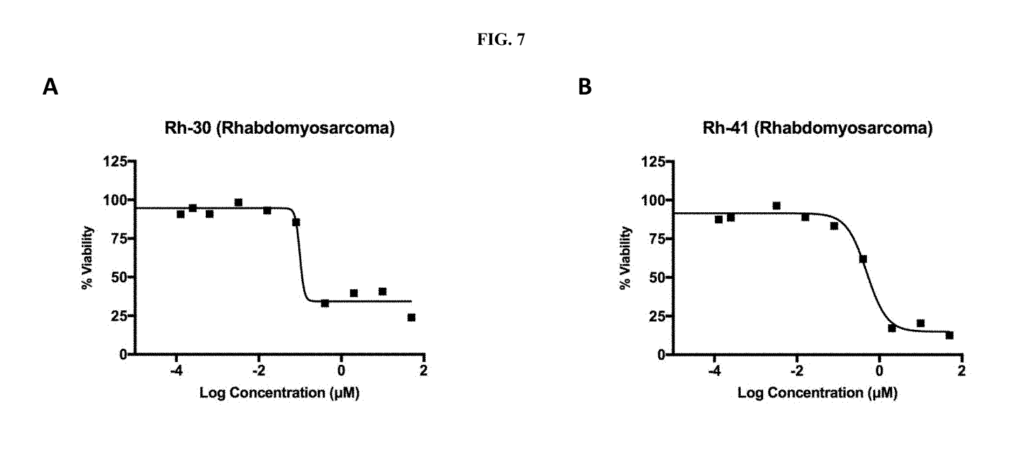

The rhabdomyosarcoma cell lines Rh-30 (FIG. 7A) and Rh-41 (FIG. 7B) were treated with ten serial dilutions of MBZ (0-50 μM) for 72 hours. The IC₅₀ for the Rh-30 cell line following treatment of MBZ was calculated to be 0.1 μM. The IC₅₀ for the Rh-41 cell line following treatment of MBZ was calculated to be 0.49 μM.

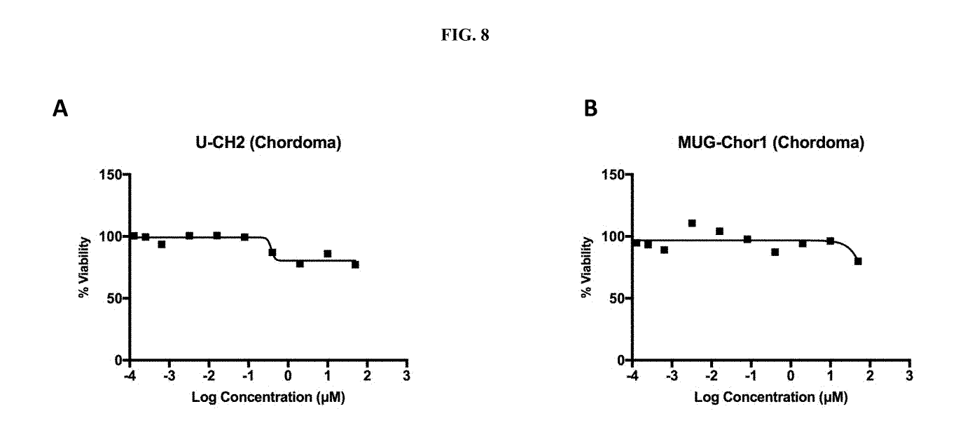

The chordoma cell lines U-CH2 (FIG. 8A) and MUG-Chor1 (FIG. 8B) were treated with MBZ for 72 hours. The concentration range was (0-50 μM) for both cell lines. A dose response curve was generated using the PRISM software, with the intent of determining an IC₅₀ for the cell lines following treatment of MBZ. Both cell lines were largely resistant to treatment up to the highest concentration, 50 μM, tested of MBZ.

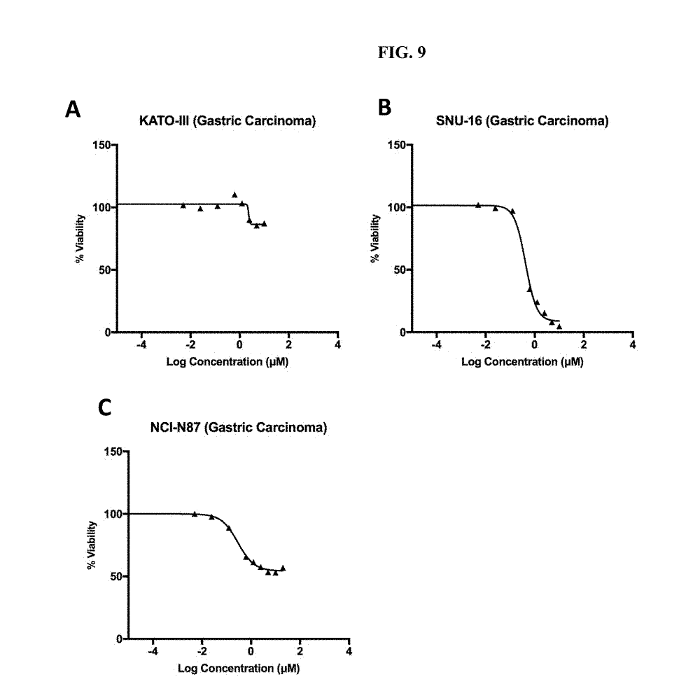

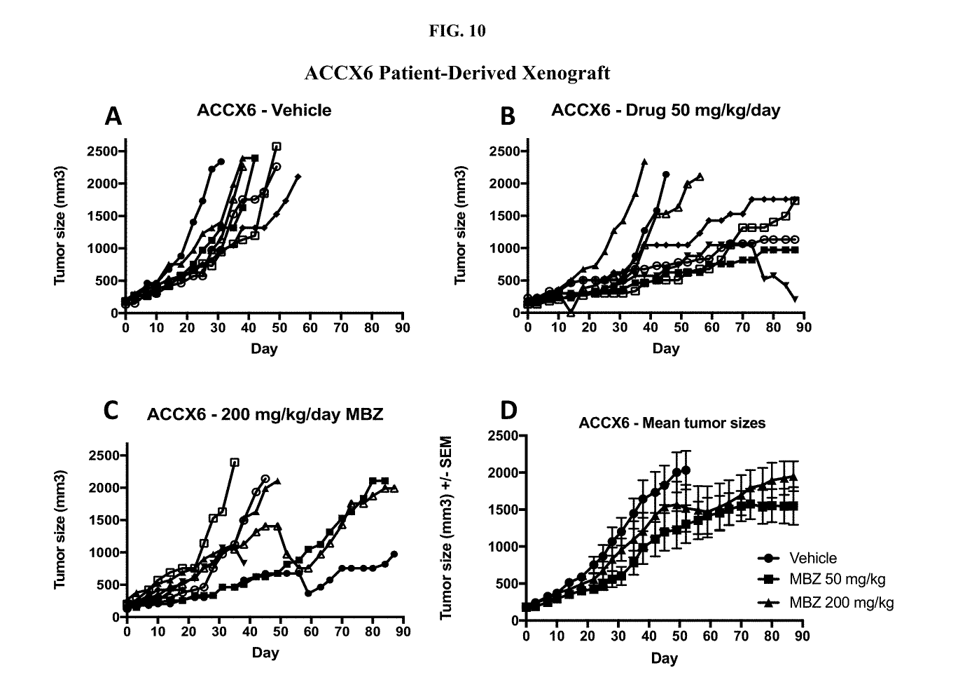

The gastric carcinoma cell lines KATO-III, NCI-N87, and SNU-16 were treated with ten serial dilutions of MBZ for 72 hours. For KATO-III (FIG. 9A) and SNU-16 (FIG. 9B) the concentration range of mebendazole tested were from 0-10 μM. The NCI-N87 (FIG. 9C) concentration range of mebendazole tested was from 0-20 μM. The IC₅₀ for the SNU-16 cell line following treatment of MBZ was calculated to be 0.42 μM. The KATO-III and NCI-N87 cell lines did not reach a 50% decrease in viability up to the highest concentration tested.

In vitro, MBZ strongly inhibited the growth of multiple rare cancer cell lines with IC₅₀ values ranging from 50 nM to 1 μM to depending on the cell line tested.

### Example 2: Adenoid Cystic Carcinoma (ACC) Patient Derived Xenograft Mice

The effectiveness of mebendazole can be assayed in vivo using a patient derived xenograft mouse model. In patient derived xenograft mice, cancerous cells isolated from a cancer patient are implanted into an immunodeficient mouse, and allowed to form tumors. The mice are then administered mebendazole, and the effect on tumor size and mouse viability is assayed. As xenograft cancers can be implanted from a variety of patient sources, the effectiveness of mebendazole treatment on multiple cancers can be assayed in this manner. Human cancer cell lines such as those tested in vitro can similarly be used in mouse xenograft tumor models.

One example of this approach looks at the effect of mebendazole on Adenoid Cystic Carcinoma (ACC). Adenoid Cystic Carcinoma (ACC) is a rare adenocarcinoma arising most commonly in the major and minor salivary glands of the head and neck. There is no known cause, and no effective treatments. While there is 90% survival at 5 years, reoccurrence is almost universal by 15 years. Metastases to the lung, liver, brain and bones are frequently fatal within a year. There are no next-generation therapies, and chemotherapy is ineffective. There are almost no survivors twenty years post diagnosis.

Despite a deepening understanding of the molecular events that lead to sustained tumor growth, there are currently no approved therapies for ACC. Identification of therapies for ACC has been hampered by the lack of ACC cell lines and transgenic mouse models. To circumvent these issues, the activity of MBZ was tested in several ACC patient-derived xenograft (PDX) models. Athymic nude mice were implanted subcutaneously with PDX tumors and treatment was initiated when tumors reached a size of 125-300 mm³. Mice were randomized into three treatment groups to receive daily treatment with vehicle or MBZ at either 50 or 200 mg/kg for the duration of the study. Tumor size was measured twice weekly. Daily oral dosing with MBZ was well tolerated with no overt toxicities. Significant anti-tumor activity was observed in 2 out of 3 ACC PDX models.

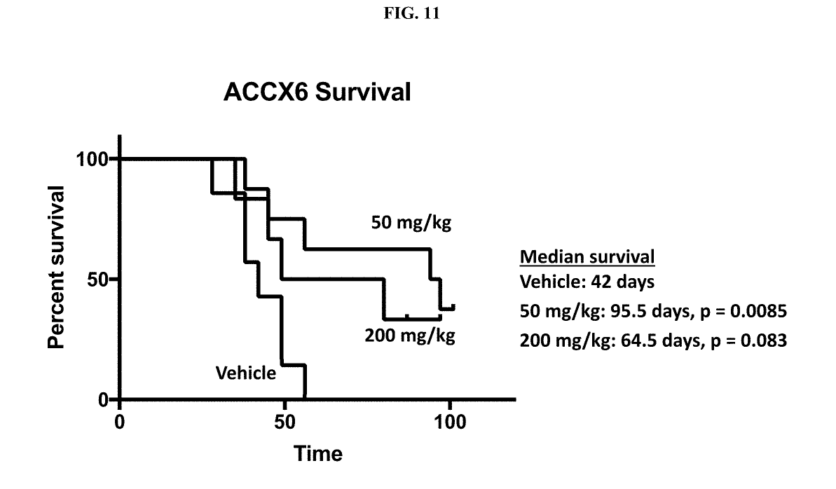

Inhibition of tumor growth was observed in the less aggressive ACCX6 PDX model at both the 50 and 200 mg/kg dose levels compared to vehicle and was accompanied by an increase in median survival (95.5 and 64.5 days vs 42 days respectively; p=00.00854 and p=0.083). FIG. 11 shows the effect of mebendazole on mouse mortality. The X-axis of FIG. 11 shows day of treatment, the Y-axis shows percent survival.

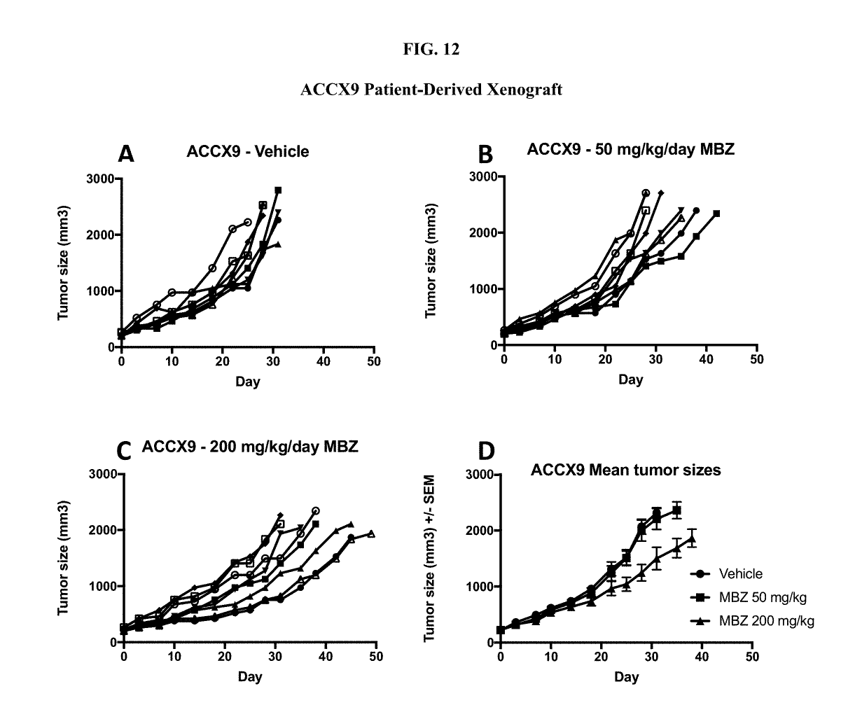

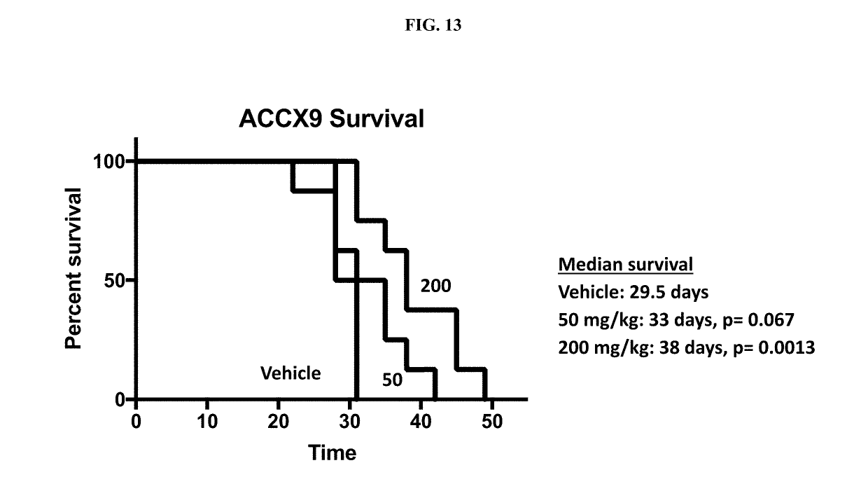

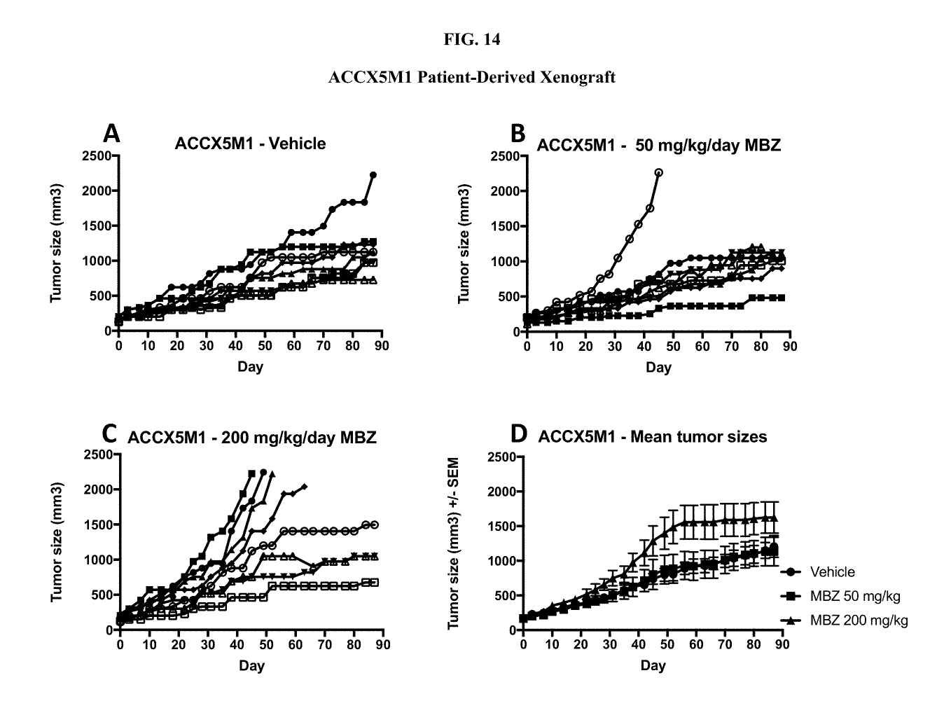

In the most aggressive ACCX9 PDX model (FIGS. 12 and 13), MBZ showed statistically significant anti-tumor activity at the highest dose of 200 mg/kg resulting in a significant increase in median survival compared to vehicle-treated mice (38 vs 29.5 days respectively, p=0.0013). Survival was also increased at 50 mg/kg MBZ but did not achieve statistical significance (33 vs 29.5 days with vehicle, p=0.067). FIG. 13 show the effect of mebendazole treatment on survival ACCX9 patient derived xenograft mice treated with vehicle, 50 mg/kg/day MBZ and 200 mg/kg/day MBZ. The X-axis shows day of treatment from 0 to 100 in increments of 20, the Y-axis shows percent survival from 0 to 100, in increments of 50.

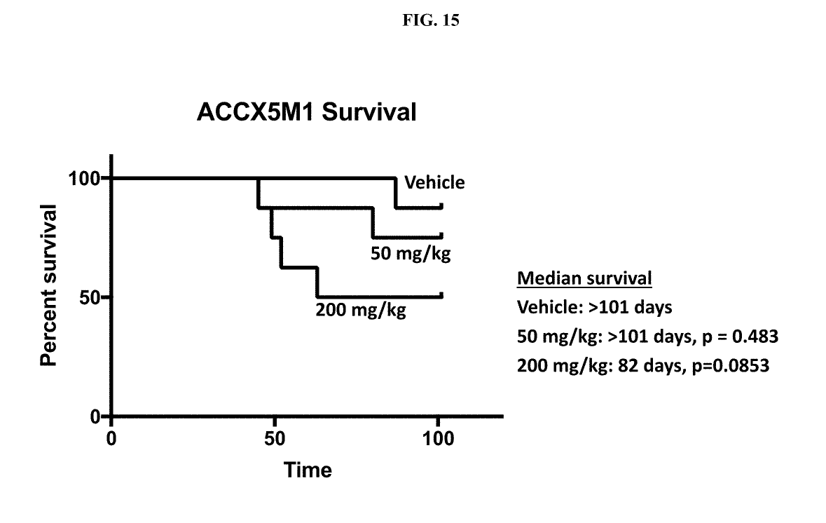

In the reportedly more refractory ACCX5M1 PDX model, treatment with MBZ did not inhibit tumor growth. FIG. 15 shows effect of mebendazole treatment on survival of ACCX5M1 patient derived xenograft mice treated with vehicle, 50 mg/kg/day MBZ and 200 mg/kg/day MBZ. The X-axis shows day of treatment from 0 to 100 in increments of 20, the Y-axis shows percent survival from 0 to 100, in increments of 50.

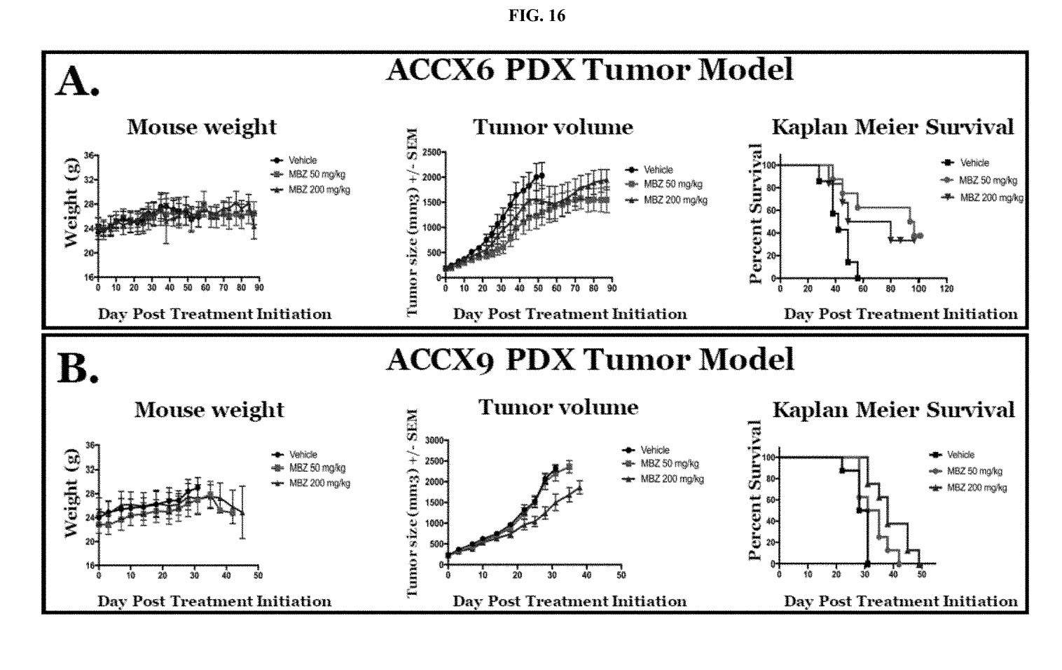

Identification of therapies for ACC has been hampered by the lack of ACC cell lines and transgenic mouse models. To circumvent these issues, the activity of MBZ was tested in several ACC patient-derived xenograft (PDX) models. Mice were implanted subcutaneously with PDX tumors and treatment was initiated when tumors reached a size of 125-300 mm³. Mice were randomized into three treatment groups to receive daily treatment with vehicle or MBZ at either 50 or 200 mg/kg for the duration of the study. Daily oral dosing with MBZ was well tolerated with no overt toxicities. Significant anti-tumor activity was observed in 2 out of 3 ACC PDX models. In the most aggressive ACCX9 PDX model, MBZ showed statistically significant anti-tumor activity at the highest dose of 200 mg/kg resulting in a significant increase in median survival compared to vehicle-treated mice (38 vs 29.5 days respectively, p=0.0013). Inhibition of tumor growth was also observed in the less aggressive ACCX6 PDX model at both the 50 and 200 mg/kg dose levels compared to vehicle and was accompanied by an increase in median survival (95.5 and 64.5 days vs 42 days respectively; p=00.0085 and p=0.083). In the reportedly more refractory ACCX5M1 PDX model, treatment with MBZ did not inhibit tumor growth. Data are shown in FIG. 16.

Tumor weight and tumor size was measured every three days once treatment with MBZ was initiated. Inhibition of tumor growth was observed in the ACCX6 PDX model (FIG. 16 (A)) at both the 50 and 200 mg/kg dose levels compared to vehicle and was accompanied by a decrease in tumor volume and an increase in median survival (95.5 and 64.5 days vs 42 days respectively; p=00.0085 and p=0.083). In the more aggressive ACCX9 PDX model (FIG. 16 (B)), MBZ showed statistically significant anti-tumor activity at the highest dose of 200 mg/kg resulting in a significant increase in median survival compared to vehicle-treated mice (38 vs 29.5 days respectively, p=0.0013) and a decrease in tumor volume.

Overall, the in vitro and in vivo results suggest that MBZ is a novel therapeutic option for the treatment of rare cancers including adenoid cystic carcinoma (ACC). In vivo, significant anti-tumor activity was observed in two out of the three ACC PDX models tested. Treatment with MBZ resulted in a significant increase in survival and a delay in tumor growth in both the ACCX6 and ACCX9 PDX tumor models.

### Example 3: Assessment of the Synergistic Activity of MBZ in Combination with Chemotherapeutic Agents in Rare Cancers

The FDA-approved anti-helminthic drug mebendazole (MBZ) has been reported to affect several biological pathways involved in tumor growth and metastasis. Based on this activity, multiple rare cancer tumor cell lines including, but not limited to, neuroblastoma, carcinoid tumors, esophageal adenocarcinoma, gastrointestinal stromal tumors, leiomyosarcoma, pheochromocytoma, and mesothelioma were analyzed for their susceptibility to MBZ treatment alone or in combination with standard chemotherapeutic agents in vitro. Cultured cells exposed to increasing concentrations of MBZ alone showed a significant decrease in cell viability with IC50s ranging from 0.09-2.2 μM.

The results of the ex vivo 3D assay using gastric cancer primary cells (see Example 5 below) along with the in vitro data generated using cell lines indicate that MBZ may represent an effective anti-cancer agent across multiple rare cancer indications. However, since tumor cells can often evade or develop resistance to single agent therapy, the potential for synergistic combinations with chemotherapeutic agents was also explored in vitro.

For in vitro experiments, cells were plated in triplicate and treated with a range of concentrations of MBZ and relevant chemotherapeutic agents in a complete Latin square. Combination indexes (CI) were determined based on the method described by Chou and Talalay (Cancer Res. 2010 Jan. 15; 70(2):440-6. doi: 10.1158/0008-5472.CAN-09-1947. Epub 2010 Jan. 12). Several synergistic drug combinations were identified with CI values ranging from 0.008 to 0.428 (CI<0.9 indicates synergy). Overall, these results show that synergistic combinations of MBZ and chemotherapy agents are a promising strategy for the treatment of rare cancers.

The activity of MBZ in combination with additional chemotherapeutic agents was assessed in vitro using rare cancer cell lines.

Cells were plated in triplicate and treated with a range of 5 concentrations of MBZ in combination with 5 concentrations of chemotherapeutic agents in a complete Latin square. Following a 72 hour incubation, cell viability was determined using Cell Titer Glo. The Combination index (CI) was determined based on the method described by Chou and Talalay (2010) with a CI value less than 0.9 indicating synergy.

For each cell line tested, synergistic drug combinations were defined as combinations where multiple pairings (greater than or equal to 3) produced a CI<0.9. In each instance where significant synergy was observed, it is documented by Yes and the CI range for the concentration pairings producing a synergistic effect on cytotoxicity is indicated.

The CI of MBZ and an additional therapeutic agent such as a chemotherapeutic agent or a small molecule is shown in tables 5-20 below.

TABLE 5
Assessment of the synergistic activity of MBZ in combination
with chemotherapeutic agents in adrenocortical carcinoma.

| Chemotherapeutic | H295R | SW13 |
|---|---|---|
| Cisplatin | Yes- (0.544-0.771) | Yes- (0.410-0.883) |
| Doxorubicin | No- (0.689-0.697) | Yes- (0.771-0.850) |
| Etoposide | No | Yes- (0.618-0.710) |

MBZ was tested in combination with Cisplatin, Doxorubicin or Etoposide in the adrenocortical carcinoma cell lines H295R and SW13. Synergy between MBZ was observed in both H295R and SW13 adrenocortical carcinoma cells. Synergy between Doxorubicin and MBZ was observed in SW13 adrenocortical carcinoma cells. Synergy between Etoposide and MBZ was observed in SW13 adrenocortical carcinoma cells.

TABLE 6
Assessment of the synergistic activity of MBZ in combination
with chemotherapeutic agents in carcinoid tumors.

| Chemotherapeutic | STC-1 |
|---|---|
| Cyclophosphamide | Yes- (0.226-0.784) |
| 5-FU | Yes- (0.458-0.850) |
| Cisplatin | Yes- (0.441-0.785) |
| Doxorubicin | Yes- (0.419-0.829) |
| Etoposide | Yes- (0.253-0.893) |

MBZ was tested in combination with Cyclophosphamide, 5-Fluorouracil (5-FU), Cisplatin, Doxorubicin or Etoposide in the carcinoid tumor cell line STC-1. MBZ showed synergy in combination with 4-HC, 5-FU Cisplatin, Doxorubicin or Etoposide in the carcinoid tumor cell line STC-1. Cyclophosphamide is broken down into the metabolically active form of the drug, 4 hydroperoxycyclophosphamide (4-HC), in the body. Accordingly, in vitro experiments testing Cyclophosphamide activity were performed with 4-HC.

TABLE 7
Assessment of the synergistic activity of MBZ in combination
with chemotherapeutic agents in cholangiocarcinoma.

| Chemotherapeutic | CCC-5 | TFK-1 |
|---|---|---|
| 5-FU | Yes- (0.252-0.878) | No |
| Cisplatin | Yes- (0.121-0.716) | Yes- (0.405-0.877) |
| Gemcitabine | Yes- (0.394-0.855) | No |
| Oxaliplatin | Yes- (0.060-0.631) | No |

MBZ was tested in combination with 5-FU, Cisplatin, Gemcitabine or Oxaliplatin in the cholangiocarcinoma cell lines CCC-5 and TFK-1. MBZ showed synergy with Cisplatin in both CCC-5 and TFK-1 cholangiocarcinoma cells. MBZ showed synergy with 5-FU, Gemcitabine and Oxaliplatin in CCC-5 cholangiocarcinoma cells.

TABLE 8
Assessment of the synergistic activity of MBZ in combination
with chemotherapeutic agents in chordoma.

| Chemotherapeutic | UM-Chor1 |
|---|---|
| Erlotinib | No |

MBZ was tested in combination with Erlotinib in UM-Chor1 chordoma cell line. No synergy was observed.

TABLE 9
Assessment of the synergistic activity of MBZ in combination
with chemotherapeutic agents in colorectal carcinoma.

| Chemotherapeutic | HCT116 | Colo-205 |
|---|---|---|
| 5-FU | Yes- (0.285-0.860 | Yes- (0.528-0.748) |
| Etoposide | Yes- (0.607-0.890) | Yes- (0.293-0.707) |
| Irinotecan | Yes- (0.545-0.793) | Yes- (0.502-0.819) |
| Oxaliplatin | Yes- (0.493-0.728) | Yes- (0.266-0.712) |

MBZ was tested in combination with 5-FU, Etoposide, Irinotecan or Oxaliplatin in HCT116 and Colo-205 colorectal carcinoma cell lines. Synergy between MBZ and 5-FU, Etoposide, Irinotecan or Oxaliplatin was observed in both HCT116 and Colo-205 cells.

TABLE 10
Assessment of the synergistic activity of MBZ in combination
with chemotherapeutic agents in esophageal carcinoma.

| Chemotherapeutic | Flo-1 | OE-33 |
|---|---|---|
| 5-FU | Yes- (0.370-0.855) | No |
| Carboplatin | Yes- (0.063-0.603) | Yes- (0.542-0.636) |
| Irinotecan | Yes- (0.439-0.871) | Yes- (0.589-0.757) |
| Oxaliplatin | Yes- (0.355-0.875) | Yes- (0.395-0.597) |
| Paclitaxel | Yes- (0.430-0.864) | Yes- (0.95-0.499) |

MBZ was tested in combination with 5-FU, Carboplatin, Irinotecan, Oxaliplatin or Paclitaxel in Flo-1 and OE-33 esophageal carcinoma cell lines. Synergy between 5-FU and MBZ was observed in Flo-1 esophageal carcinoma cells. Synergy between Carboplatin, Irinotecan, Oxaliplatin or Paclitaxel and MBZ was observed in both Flo-1 and OE-33 esophageal carcinoma cells.

TABLE 11
Assessment of the synergistic activity of MBZ in combination
with chemotherapeutic agents in gastric carcinoma.

| Chemotherapeutic | Kato-III | SNU16 |
|---|---|---|
| 5-FU | Yes- (0.073-0.558) | Yes- (0.429-0.827) |
| Carboplatin | No | Yes- (0.409-0.783) |
| Irinotecan | No | Yes- (0.635-0.871) |
| Oxaliplatin | Yes- (0.587-0.897) | Yes- (0.571-0.883) |
| Paclitaxel | Yes- (0.193-0.508) | Yes- (0.425-0.883) |

MBZ was tested in combination with 5-FU, Carboplatin, Irinotecan, Oxaliplatin or Paclitaxel in Kato-III and SNU16 gastric carcinoma cell lines. Synergy between MBZ and 5-FU, Oxaliplatin or Paclitaxel was observed in both Kato-III and SNU16 gastric carcinoma cells. Synergy between MBZ and Carboplatin or Irinotecan was observed in SNU16 gastric carcinoma cells.

TABLE 12
Assessment of the synergistic activity of MBZ in combination with
small molecule agents in gastrointestinal stromal tumors.

| Chemotherapeutic | GIST-1 |
|---|---|
| Imatinib | Yes- (0.627-0.875) |
| Abemaciclib | Yes- (0.390-0.701) |

MBZ was tested in combination with Imatinib or Abemaciclib in the GIST-1 gastrointestinal stromal tumor cell line. Synergy between MBZ and Imatinib or Abemaciclib was detected.

TABLE 13
Assessment of the synergistic activity of MBZ in combination
with chemotherapeutic agents in leiomyosarcoma.

| Chemotherapeutic | SK-LMS-1 | SK-UT-1 | SK-UT-1B |
|---|---|---|---|
| Docetaxel | No | Yes- (0.260-0.884) | Not determined |
| Doxorubicin | Yes- (0.514-0.872) | No | Yes- (0.716-0.887) |
| Gemcitabine | Yes- (0.418-0.861) | Yes- (0.385-0.802) | Yes- (0.410-0.893) |
| Ifosamide | No | Yes- (0.317-0.708) | Not determined |

Docetaxel, Doxorubicin, Gemcitabine or Ifosamide SK-LMS-1, SK-UT-1 and SK-UT-1B leiomyosarcoma cell lines. Synergy between MBZ and Docetaxel was detected in SK-UT-1 leiomyosarcoma cells. Synergy between MBZ and Doxorubicin was detected in SK-LMS-1 and SK-UT-1B leiomyosarcoma cells. Synergy between MBZ and Gemcitabine was detected in SK-LMS-1, SK-UT-1 and SK-UT-1B leiomyosarcoma cells. Synergy between MBZ and Ifosamide was detected in SK-UT-1 leiomyosarcoma cells.

TABLE 14
Assessment of the synergistic activity of MBZ in
combination with chemotherapeutic agents and a small
molecule agent in hepatocellular carcinoma.

| Chemotherapeutic | HepG2 | HepG2-C3A |
|---|---|---|
| Gemcitabine | Yes- (0.012-0.796) | No |
| Oxaliplatin | Yes- (0.036-0.834) | No |
| Sorafenib | Yes- (0.096-0.483) | Yes- (0.157-0.715) |

MBZ was tested in combination with Gemcitabine, Oxaliplatin or Sorafenib in HepG2 and HepG2-C3A hepatocellular carcinoma cell lines. Synergy between MBZ and Gemcitabine or Oxaliplatin was detected in HepG2 hepatocellular carcinoma cells. Synergy between MBZ and Sorafenib was detected in HepG2 and HpG2-C3A hepatocellular carcinoma cells.

TABLE 15
Assessment of the synergistic activity of MBZ in combination
with chemotherapeutic agents in mesothelioma.

| Chemotherapeutic | JU77 | LO68 | MSTO-211H |
|---|---|---|---|
| Carboplatin | Yes- (0.539-0.841) | Yes- (0.574-0.865) | No |
| Doxorubicin | Yes- (0.458-0.820) | Yes- (0.433-0.875) | Yes- (0.288-0.700) |
| Gemcitabine | Yes- (0.362-0.712) | Yes- (0.525-0.845) | Yes- (0.086-0.796) |
| Vinorelbine | Yes- (0.325-0.799) | Yes- (0.445-0.872) | Yes- (0.087-0.885) |

MBZ was tested in combination with Carboplatin, Doxorubicin, Gemcitabine or Vinorelbine in JU77, LO68 and MSTO-211H mesothelioma cell lines. Synergy between MBZ and Carboplatin was detected in JU77 and LO68 mesothelioma cells. Synergy between MBZ and Doxorubicin, Gemcitabine or Vinorelbine was detected in JU77, LO68 and MSTO-211H mesothelioma cells.

TABLE 16
Assessment of the synergistic activity of MBZ in combination
with chemotherapeutic agents in neuroblastoma.

| Chemotherapeutic | CHP-212 | SK-N-AS |
|---|---|---|
| Cyclophosphamide | Yes- (0.569-0.860) | No |
| Carboplatin | Yes- (0.587-0.876) | Yes- (0.433-0.891) |
| Cisplatin | Yes- (0.653-0.896) | Yes- (0.141-0.746) |
| Doxorubicin | Yes- (0.646-0.883) | Yes- (0.267-0.849) |
| Etoposide | Yes- (0.647-0.891) | No |

MBZ was tested in combination with Cyclophosphamide, Carboplatin, Cisplatin, Doxorubicin or Etoposide in CHP-212 and SK-N-AS neuroblastoma cell lines. Synergy between Cyclophosphamide or Etoposide and MBZ was detected in CHP-212 neuroblastoma cells. Synergy between MBZ and Carboplatin, Cisplatin or Doxorubicin was detected in both CHP-212 and SK-N-AS neuroblastoma cells.

TABLE 17
Assessment of the synergistic activity of MBZ in combination
with chemotherapeutic agents in ovarian carcinoma.

| Chemotherapeutic | COV362 | TOV-112D |
|---|---|---|
| Carboplatin | Yes- (0.553-0.860) | Yes- (0.506-0.845) |
| Cisplatin | Yes- (0.578-0.878) | Yes- (0.497-0.860) |
| Docetaxel | Yes- (0.399-0.867) | No |
| Paclitaxel | Yes- (0.318-0.889) | Yes- (0.367-0.839) |

MBZ was tested in combination with Carboplatin, Cisplatin, Docetaxel or Paclitaxel in COV362 and TOV-112D ovarian carcinoma cell lines. Synergy between MBZ and Docetaxel was detected in Cov362 ovarian carcinoma cells. Synergy between MBZ and Carboplatin, Cisplatin or Paclitaxel was detected in both COV362 and TOV-112D ovarian carcinoma cells.

TABLE 18
Assessment of the synergistic activity of MBZ in combination
with chemotherapeutic agents in pancreatic carcinoma.

| Chemotherapeutic | BxPC-3 | Hs766t |
|---|---|---|
| 5-FU | Yes- (0.448-0.792) | Yes- (0.170-0.877) |
| Cisplatin | Yes- (0.469-0.762) | Yes- (0.658-0.847) |
| Docetaxel | Yes- (0.020-0.793) | Yes- (0.443-0.832) |
| Gemcitabine | Yes- (0.502-0.770) | Yes- (0.149-0.874) |
| Irinotecan | Yes- (0.659-0.793) | No |
| Oxaliplatin | Yes- (0.455-0.758) | No |
| Paclitaxel | Yes- (0.341-0.845) | No |

MBZ was tested in combination with 5-FU, Cisplatin, Docetaxel, Gemcitabine, Irinotecan, Oxaliplatin or Paclitaxel in BxPC-3 and Hs766t pancreatic carcinoma cell lines. Synergy between MBZ and Irinotecan, Oxaliplatin or Paclitaxel was detected in BxPC-3 pancreatic carcinoma cells. Synergy between MBZ and 5-FU, Cisplatin, Docetaxel or Gemcitabine was detected in both BxPC-3 and Hs766t pancreatic carcinoma cells.

TABLE 19
Assessment of the synergistic activity of MBZ in combination with
chemotherapeutic agents in pheochromocytoma/paraganglioma.

| Chemotherapeutic | PC12 |
|---|---|
| Cyclophosphamide | Yes- (0.388-0.879) |
| Dacarbazine | Yes- (0.246-0.785) |
| Vincristine | Yes- (0.274-0.871) |

MBZ was tested in combination with Cyclophosphamide, Dacarbazine or Vincristine in the PC12 pheochromocytoma/paraganglioma cell line. Synergy between MBZ and Cyclophosphamide, Dacarbazine or Vincristine was detected in PC12 pheochromocytoma/paraganglioma cells.

TABLE 20
Assessment of the synergistic activity of MBZ in combination
with chemotherapeutic agents in small cell lung carcinoma.

| Chemotherapeutic | DMS-114 | SW-1271 |
|---|---|---|
| Etoposide | Yes- (0.551-0.832) | Yes- (0.535-0.899) |
| Cisplatin | Yes- (0.223-0.837) | Yes- (0.275-0.763) |

MBZ was tested in combination with Etoposide or Cisplatin in DMS-114 and SW-1271 small cell lung carcinoma cell lines. Synergy between MBZ and Etoposide was detected in both DMS-114 and SW-1271 small cell lung carcinoma cells. Synergy between MBZ and Cisplatin was detected in both DMS-114 and SW-1271 small cell lung carcinoma cells.

### Example 4: Encapsulation of MBZ in an HA-PLGA Nanoparticle

MBZ was formulated in an hyaluronic acid (HA)-poly(lactic-co-glycolic acid (PLGA) nanoparticle to provide superior solubility and delivery to cancer cells. HA on the nanoparticle binds to CD44, a transmembrane glycoprotein expressed by many cancer cells.

In Table 21 below, cells were plated in triplicate and treated with a range of 9 concentrations of MBZ and 9 concentrations of the molar equivalent amount of MBZ contained within a HA-PLGA nanoparticle (HA-PLGA-MBZ). Following a 72 hour incubation, cell viability was determined using Cell Titer Glo. An IC₅₀ for each treatment was established using the PRISM software.

TABLE 21
Encapsulation of MBZ in a HA-PLGA nanoparticle
does not impact its biological activity.

| | MBZ IC₅₀ | HA-PLGA-MBZ IC₅₀ |
|---|---|---|
| Cell Line | (mM) | (mM) |
| COLO-205 | 0.27 | 0.41 |
| SW-1271 | >50 | >50 |
| NCI-H295R | 0.33 | 0.72 |
| DMS-114 | 0.80 | 0.71 |
| HCT-116 | 0.32 | 0.26 |
| PC-12 | 1.03 | 1.49 |
| SK-UT-1 | 0.26 | 0.21 |
| SNU-16 | 0.11 | 0.10 |
| SKOV-3 | 0.68 | 0.42 |
| COV-362 | 0.33 | 0.31 |
| CHP-212 | 0.13 | 0.10 |

Synergy between the nanoparticle formulation of MBZ (HA-PLGA-MBZ) and chemotherapeutic agents was observed in representative cell lines.

In table 22 below, cells were plated in triplicate and treated with a range of 5 concentrations of MBZ or HA-PLGA-MBZ nanoparticles containing a molar equivalent of MBZ, and 5 concentrations of relevant chemotherapeutic agents in a complete Latin square. Combination indexes (CI) were determined based on the method described by Chou-Talalay with a CI value less than 0.9 indicating synergy. For each cell line tested, synergistic drug combinations were defined as combinations where multiple pairings (greater than or equal to 3) produced a CI<0.9. Numbers in parentheses represent the range of synergistic CI values for each cell line and drug combination.

TABLE 22
In vitro activity of naked MBZ and HA-PLGA-MBZ nanoparticles
used in combination with chemotherapy agents is comparable
and produces similar amounts of synergy.

| Cell Line and | | |
|---|---|---|
| Chemotherapeutic | MBZ | HA-PLGA-MBZ |
| CHP-212 + 4-HC | Yes- (0.569-0.860) | Yes- (0.461-0.827) |
| SNU16 + 5-FU | Yes- (0.223-0.837) | Yes- (0.424-0.832) |
| PC12 + Vincristine | Yes- (0.274-0.870) | Yes- (0.232-0.870) |

### Example 5: Efficacy of MBZ in an Ex Vivo Assay 3D Assay Using Gastric Cancer Primary Cells

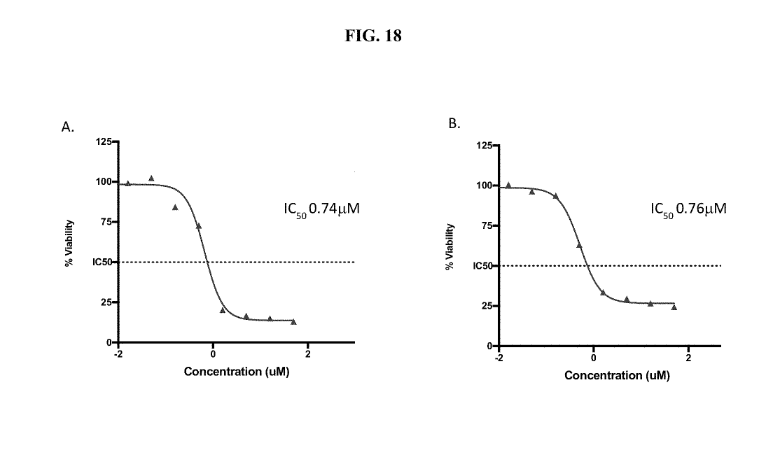

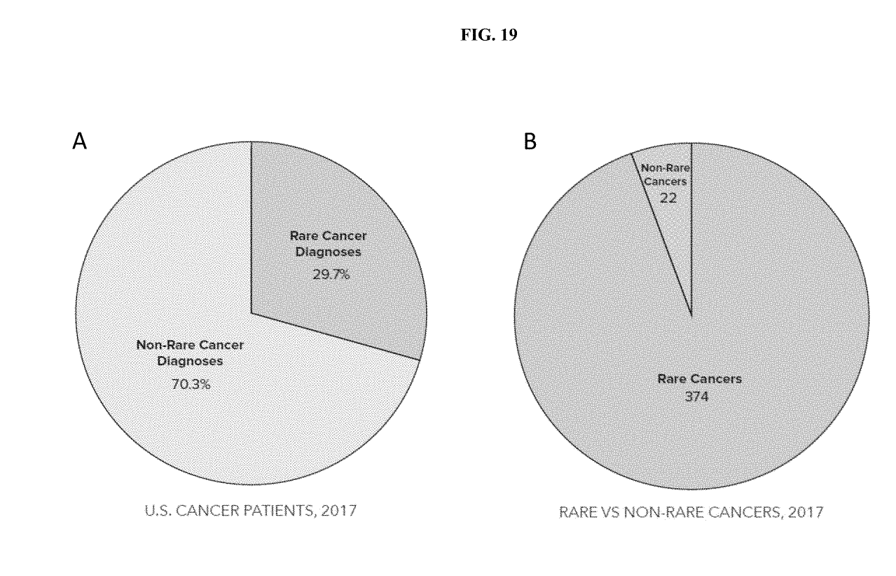

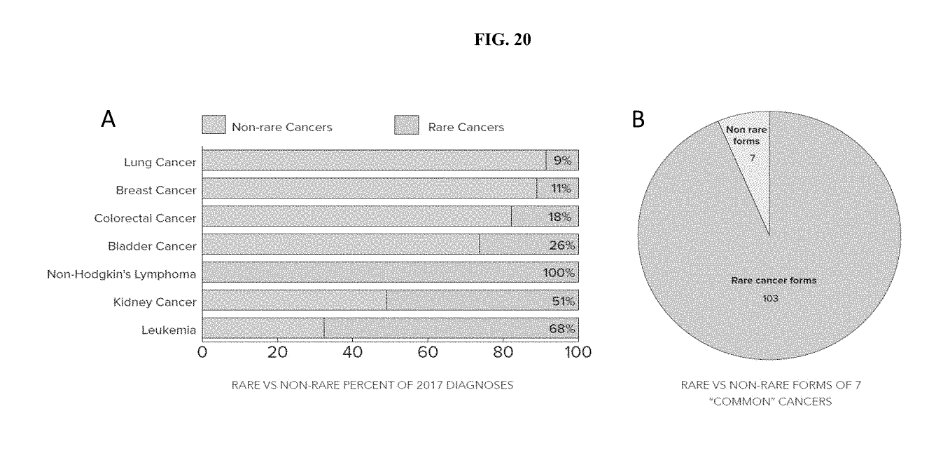

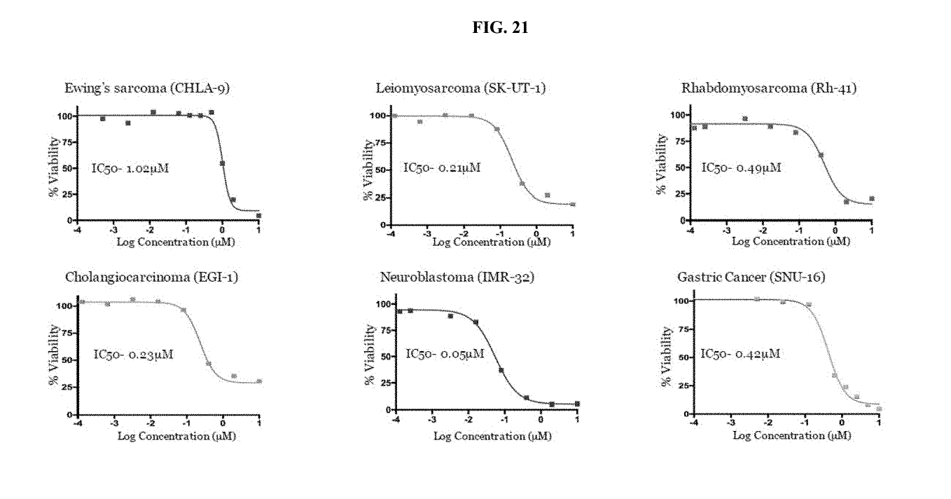

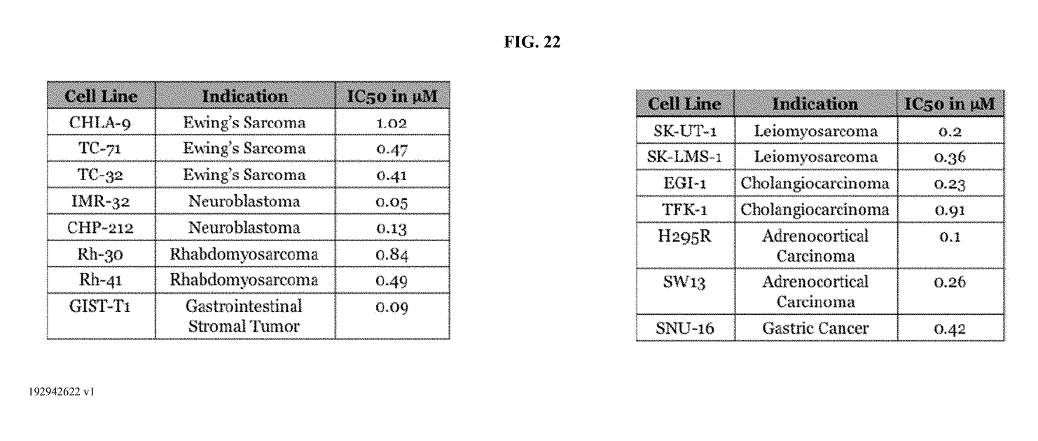

To evaluate the efficacy of MBZ in an additional model system, an ex vivo 3D assay using gastric cancer primary cells isolated from two patient-derived xenograft models (A. GA0033 and B. GA3155) was performed. Cells were seeded in 3D cultures and treated for seven days with MBZ at nine different doses in triplicate. Cell viability was determined using Cell Titer Glo. An IC₅₀ was established using the PRISM software. IC₅₀ s were determined to be 0.74 and 0.76 mM indicative of significant potency. Percent viability versus concentration for both xenografts is shown in FIG. 18. IC₅₀s were determined to be 0.74 and 0.76 μM indicative of significant potency. These results along with the in vitro data generated using cell lines suggest that MBZ may represent an effective anti-cancer agent across multiple rare cancer indications.

### Example 6: Incidence of Rare Cancers

Rare cancers are an understudied and deadly public health problem. Estimates for the percentage of cancer diagnoses that are rare vary depending upon the source cited and the definition of what constitutes a rare cancer. Following the NCI definition of a rare disease as affecting fewer than 15 persons per 100,000 per year a cancer affecting 45,691 or fewer is classified as rare. Over 300 rare cancers exist for which there are few treatment options beyond surgical resection representing a significant unmet medical need. Treatment options are currently being explored for several rare cancer indications including repurposing of drugs clinically approved for other indications.

Some of the most severely affected cancer patients are minorities, veterans, those who reside in rural areas, those of the lowest socioeconomic status (SES), those of color, and those who are pediatric patients below the age of 19. At least 64 forms of cancer disproportionately affect veterans and are correlated with service-related exposures such as burn pits and Agent Orange. Between 44-52 of these are defined as rare cancers. Minorities and women are disproportionately affected by dozens of cancers, many of them rare, and frequently face significant economic and social burdens to receiving treatment and participating in clinical trials. Pediatric cancer research receives 4% or less of total NCI funding. Most NCI funding supports discovery-stage basic research and not translational science. All pediatric cancers are rare.

Despite advances in the understanding of the factors involved in promoting tumor growth, up to 87% of rare cancer patients have no treatment options beyond surgical resection, radiation and/or traditional chemotherapy. Repurposing of drugs for use in oncology represents an attractive strategy for the rapid development of therapies that address the significant unmet medical need that remains for rare cancers.

### Example 7: Immunohistochemistry Analysis Indicates that Myb Levels do not Correlate with MBZ Activity In Vivo

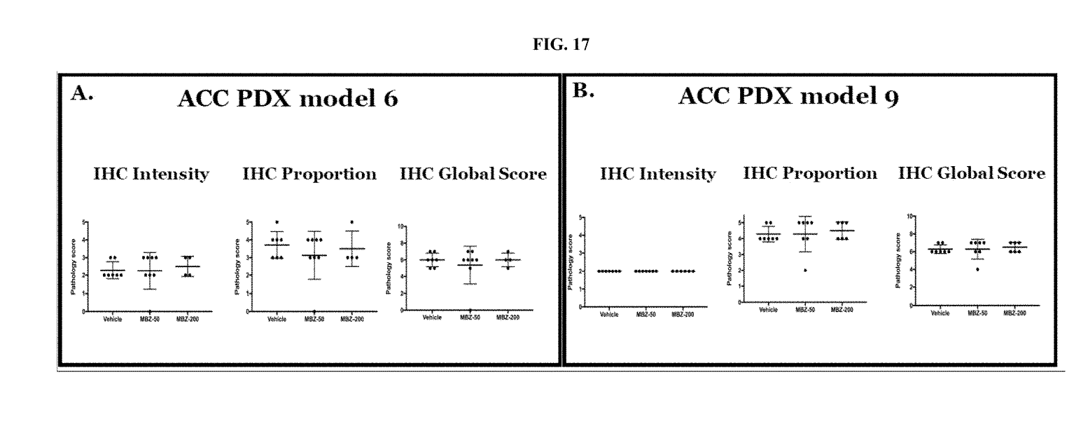

It has been suggested MBZ may act as a Myb degrader and thereby affect the growth of adenoid cystic carcinoma (ACC) tumors. To investigate this possibility, tumors from mice at the end of the treatment period were collected and snap frozen at −80° C. Immunohistochemical analysis for Myb protein levels was performed on individual tumors from each of the groups for each adenoid cystic carcinoma (ACC) PDX model. A pathology score was created on a scale of 1-5 to score each tumor for the intensity of Myb staining as well as for the degree to which Myb protein was detected throughout the tumor (IHC proportion). The global score represents a combination of the intensity and proportion scores. The data is shown in FIG. 17.

Despite previous reports suggesting that MBZ induces the proteosomal degradation of the proto-oncogenic transcription factor Myb, immunohistochemistry of tumors from the PDX study showed no correlation between Myb levels and anti-tumor activity.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

1. A method of treating a cancer in a subject in need thereof, comprising administering to the subject a therapeutically effective amount of a composition comprising methyl N-(6-benzoyl-1H-benzimidazol-2-yl)carbamate (mebendazole).

2-3. (canceled)

4. The method according to claim 1, wherein the composition comprising mebendazole comprises a salt.

5. The method according to claim 4, wherein the mebendazole salt comprises a mebendazole hydrochloride salt, a mebendazole hydrobromide salt, a mebendazole maleate salt, a mebendazole-glutarate salt or a mebendazole monomethyl oxalate salt.

6. The method according claim 1, wherein the composition comprising mebendazole comprises a crystal form or a polymorph of mebendazole.

7. The method according to claim 6, wherein the polymorph comprises a polymorph A of mebendazole, a polymorph B of mebendazole, a polymorph C of mebendazole or a combination thereof.

8. (canceled)

9. The method according claim 1, wherein the composition comprising mebendazole further comprises a nanoparticle.

10. The method according to claim 9, wherein the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle.

11. The method according to claim 10, wherein the polymer-based nanoparticle comprises a multiblock copolymer, a diblock copolymer, a polymeric micelle or a hyperbranched macromolecule.

12. (canceled)

13. The method according to claim 10, wherein the polymer-based nanoparticle comprises a poly(lactic-co-glycolic acid) PLGA polymer.

14-15. (canceled)

16. The method according to claim 10, wherein the nanoparticle further comprises a targeting agent.

17. The method according to claim 16, wherein the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

18. The method according to claim 16, wherein the targeting agent comprises hyaluronic acid (HA).

19. The method according to claim 16, wherein the targeting agent binds to the surface of a cell of the cancer of the subject.

20. The method according to claim 1, wherein the cancer comprises a colorectal cancer, a gastric cancer, a brain cancer, colon cancer, a breast cancer, a liver cancer, a lung cancer, a pancreatic cancer or a renal cancer.

21. (canceled)

22. The method according to claim 1, wherein the cancer is a rare cancer.

23. The method according to claim 22, wherein the cancer is a blastoma, a sarcoma, a carcinoma, a neuroendocrine cancer, a mesothelioma, a chordoma, a thymic cancer, a gastrointestinal stromal tumor or a pheochromocytoma.

24. The method according to claim 23, wherein the blastoma comprises a neuroblastoma or a glioblastoma.

25. The method according to claim 23, wherein the sarcoma comprises an Ewing's sarcoma, a leiomyosarcoma, an angiosarcoma or a rhabdomyosarcoma.

26. The method according to claim 23, wherein the carcinoma comprises an adenoid cystic carcinoma (ACC), a uterine serous carcinoma, an adrenocortical carcinoma, a gastric carcinoma, a cholangiocarcinoma, a colorectal carcinoma, an esophageal carcinoma, a hepatocellular carcinoma, a pancreatic carcinoma, a small cell lung carcinoma, an ovarian carcinoma or a thymic carcinoma.

27. (canceled)

28. The method according to claim 23, wherein the thymic cancer comprises a thymoma or a thymic carcinoma.

29. The method according to claim 23, wherein the neuroendocrine cancer comprises a carcinoid tumor or a thymic cancer.

30. (canceled)

31. The method according to claim 1, wherein the cancer is a stage 0 or stage 1 pre-metastatic cancer, a stage 2 or stage 3 cancer that has spread to nearby tissues and lymph nodes, or a stage 4 advanced or metastatic cancer.

32-33. (canceled)

34. The method according to claim 1, wherein the subject is a mammal, a non-human primate or a human.

35-36. (canceled)

37. The method according to claim 1, wherein the composition comprising mebendazole is suitable for systemic, oral or parenteral administration.

38. The method according to claim 37, wherein the administration comprises at least 30 mg, 50 mg, 100 mg, 200 mg, 300 mg, 400 mg, 500 mg, 600 mg, 700 mg, 800 mg 900 mg, 1000 mg, 1100 mg, 1200 mg, 1300 mg, 1400 mg, 1500 mg, 1600 mg, 1700 mg, 1800 mg, 1900 mg or 2000 mg of mebendazole per day.

39. (canceled)

40. The method or according to of claim 37, wherein the parenteral administration comprises intramuscular, subcutaneous or intravenous administration.

41. The method according to claim 37, wherein the administration occurs once a day, twice a day, three times a day or four or more times a day.

42. The method or according to claim 1, wherein the method of treatment further comprises an additional cancer treatment.

43. (canceled)

44. The method according to claim 42, wherein the additional cancer treatment comprises a surgical procedure to remove at least on tumor of the cancer, at least one dose of radiation therapy, a second chemotherapeutic agent, a combination chemotherapy, a therapeutic antibody, a chimeric antigen receptor T cell (CAR-T) therapy or a combination thereof.

45. The method according to claim 44, wherein the second chemotherapeutic agent comprises a cell cycle checkpoint inhibitor, a CDK inhibitor, an mTOR inhibitor, an immune checkpoint modulator, an antimitotic agent, a pro-apoptotic agent, a DNA damaging agent or an inhibitor of a DNA damage response pathway.

46. The method according to claim 45, wherein the CDK inhibitor comprises an inhibitor of CDK4, an inhibitor of CDK6 or an inhibitor of CDK4 and CDK6.

47. The method according to claim 45, wherein the CDK inhibitor comprises Abemaciclib, Palbociclib or Ribociclib.

48. The method according to claim 45, wherein the mTOR inhibitor comprises Rapamycin, Temsirolimus, Everolimus or Ridaforolimus.

49. The method according to claim 45, wherein the immune checkpoint modulator comprises Ipilimumab, Nivolumab, Atezolizumab or Pembrolizumab.

50. The method according to claim 44, wherein the second chemotherapeutic agent comprises Methotrexate, Afinitor, Pemetrexed, Melphalan, Pamidronate, Anastrozole, Exemestane, Bleomycin, Bosutinib, Busulfan, Vandetanib, Bicalutamide, Lomustine, Daunorubicin, Clofarabine, Cabozantinib, Dactinomycin, Cobimetinib, Cytarabine, Cytoxan, Dacarbazine, Decitabine, Daunorubicin Lipid Complex, Dexamethasone, Cytarabine Lipid Complex, Hydroxyurea, Leuprolide, Epirubicin, Oxaliplatin, Asparaginase, Estramustine, Vismodegib, Asparaginase Erwinia chrysanthemi, Amifostine, Etoposide, Flutamide, Toremifene, Panobinostat, Fulvestrant, Letrozole, Degarelix, Fludarabine, Pralatrexate Injection, floxuridine, Afatinib, Imatinib Mesylate, Carmustine, high dose Cytarabine, Eribulin, Altretamine, Topotecan, Ponatinib, Idarubicin, (Ifosfamide), Ibrutinib, Axitinib, Interferon alfa-2a, Gefitinib, Romidepsin, Ixabepilone, Ruxolitinib, Cabazitaxel Injection, Carfilzomib, Lenvatinib mesylate, Lanreotide acetate, Chlorambucil, Sargramostim, Cladribine, Trifluridine and Tipiracil, Leuprolide, Olaparib, Mitotane, Procarbazine, Radium 223 dichloride, Megestrol, Trametinib, Mesna, Strontium-89 Chloride, Mechlorethamine, Mitomycin, Vinorelbine, filgrastim, pegfilgrastim, Sorafenib, nilutamide, Pentostatin, Tamoxifen, Mitoxantrone, Sonidegib, Pegaspargase, Denileukin Diftitox, Alitretinoin, Pomalidomide, Prednisone, Aldesleukin, Mercaptopurine, Zoledronic acid, Lenalidomide, Octreotide, Octreotide, Dasatinib, Peginterferon Alfa-2b, Omacetaxin, Thioguanine, Dabrafenib), Erlotinib, Bexarotene, Temozolomide, Thiotepa, Thalidomide, TheraCys BCG, TICE BCG, Temsirolimus, Trabectedin, Bendamustine hydrochloride, Triptorelin, Arsenic trioxide, lapatinib, Valrubicin Intravesical, Bortezomib, Tretinoin, Azacitidine, Pazopanib, Teniposide, Leucovorin, Crizotinib, Capecitabine, Enzalutamide, Ziv-aflibercept, Streptozocin, Vemurafenib, Goserelin, Vorinostat, Zoledronic acid, Idelalisib, Ceritinib, Abiraterone acetate, Vindesine, Raltitrexed, Lometrexol, Satraplatin, Larotaxel, Alectinib, Ixazomib, Nilotinib, Osimertinib, Venetoclax, Enasidenib, Rucaparib, Niraparib, Copanlisib, Neratinib, Brigatinib, Midostaurin or a combination thereof.

51. The method according to claim 44, wherein the second chemotherapeutic agent comprises Paclitaxel, Docetaxel, Vinblastine, Vincristine, Cisplatin, Carboplatin, Oxaliplatin, Doxorubicin, Etoposide, Imatinib, Gemcitabine, Vinorelbine, Ifosfamide, Abemaciclib, Sorafenib, Irinotecan, 5-Fluorouracil, Dacarbazine, Trabectedin, Temozolomide, Cyclophosphamide or a combination thereof.

52. The method according to claim 44, wherein the therapeutic antibody comprises Adcetris (Brentuximab Vedotin, Ofatumumab, Bevacizumab, Tositumomab, Avelumab, Blinatumomab, Alemtuzumab, Ramucirumab, Daratumumab, Elotuzumab, Cetuximab, Obinutuzumab, Durvalumab, Trastuzumab, Obinutuzumab, Ado-trastuzumab Emtansine, Pembrolizumab, Olaratumab, Gemtuzumab Ozogamicin, Ocrelizumab, Nivolumab, Pertuzumab, Necitumumab, Catumaxomab, Catumaxomab, Rituximab, Siltuximab, Atezolizumab, Dinutuximab, Panitumumab, Ipilimumab, Denosumab, Ibritumomab Tiuxetan, Mogamulizumab or a combination thereof.

53. The method according to claim 44, wherein the combination chemotherapy comprises 7+3, ABVD, AC, AD, ADE, ADOC, BEACOPP, BEP, CAF, CAPIRI, CAPOX, CB, CBI, CEF, CEPP, CFAR, CHOP, CIM, CLAG, CLAG-M, CMC, CMF, COI, CVD, CVP, DHAP, DVD, ECF, ECX, EOF, EOX, EP, EPOCH, EPOCH+R, ESHAP, FAMTX, FC, FCR, FEC, FLAG-IDA, FLO, FLOX, FOLFIRI, FOLFOX, FOLFOXIRI, GEMOX-B, GVD, Hyper-CVAD, ICE, ICE-V, IFL, IROX, LV5FU2, LV5FU-P, MAID, MFL, MINE, MOPP, MP, MPV, MVAC, OFF, PAC, PAD, PCR, PCV, R-MPV, R-GemOx, R-CHOP, R-CVP, R-FCM, RICE, TAC, TC, TCH, TIP, TPC, TPF, VAD, VIP, VMP, VMPT, XELIRI or XELOX.

54-55. (canceled)

56. The method according to claim 44, wherein the additional cancer treatment and the composition comprising mebendazole are suitable for simultaneous administration, for sequential administration or for administration in temporal proximity.

57-58. (canceled)

59. The method according to claim 44, wherein the additional cancer treatment and the composition comprising mebendazole exhibit synergy.

60. The method according to claim 59, wherein the synergy is measured using the Chou-Talalay method in at least one cancer cell line.

61. The method to claim 60, wherein the synergy comprises a CI of less than 0.9 when measured at at least three concentrations of the additional cancer treatment and the composition comprising mebendazole in at least one cancer cell line.

62. (canceled)

63. The method according to claim 44, wherein the method or composition for use alleviates a sign or a symptom of the cancer.

64. (canceled)

65. A composition comprising a synergistic combination of mebendazole and at least one additional cancer therapeutic agent.

66-72. (canceled)

73. The composition of claim 65, wherein the at least one additional cancer therapeutic agent comprises Cisplatin, Doxorubicin, Etoposide, Cyclophosphamide, 5-FU, Gemcitabine, Oxaliplatin, Irinotecan, Vinorelbine, Dacarbazine, Vincristine, Sorafenib, Paclitaxel, Imatinib, Abemaciclib, Ifosfamide or Docetaxel.

74. The composition of claim 65, wherein the mebendazole is formulated in a nanoparticle.

75. The composition of claim 65, wherein the mebendazole and the at least one additional cancer therapeutic agent are formulated in a nanoparticle.

76-78. (canceled)

79. The composition of claim 76, wherein the nanoparticle comprises a poly(lactic-co-glycolic acid) PLGA polymer.

80-81. (canceled)

82. The composition of claim 76, wherein the nanoparticle further comprises a targeting agent.

83. (canceled)

84. The composition of claim 82, wherein the targeting agent comprises hyaluronic acid (HA).

85. (canceled)

86. A combinational therapy for treating cancer, comprising administering a therapeutically effective amount of the composition of claim 65 to a subject in need thereof.

87. A combinational therapy for treating cancer, comprising administering a synergistically effective amount of the claim 65 to a subject in need thereof.

88-90. (canceled)

91. A kit, comprising a therapeutically effective amount of a composition comprising mebendazole and instructions for use in the treatment of cancer.

92-99. (canceled)
